(19) United States
(12) Patent Application Publication
(10) Pub. No.: US 2008/0076771 A1
(43) Pub. Date: Mar. 27, 2008

(54) Thrombopoietin Receptor Agonists

(76) Inventors: Lawrence A. Reiter, Mystic, CT (US); Robert G. Linde, Old Lyme, CT (US)

Correspondence Address: PFIZER INC., PATENT DEPARTMENT, MS8260-1611, EASTERN POINT ROAD, GROTON, CT, 06340, US

(21) Appl. No.: 11/575,680

(22) PCT Filed: Sep. 12, 2005
(86) PCT No.: PCT/IB05/02892

§ 371 Date: Mar. 21, 2007

Related U.S. Application Data
(60) Provisional application No. 60/612,911, filed on Sep. 23, 2004.
(60) Provisional application No. 60/692,691, filed on Jun. 20, 2005.

(51) Int. Cl. A61K 31/506; A61K 31/501; A61K 31/53; A61P 7/00; C07D 413/12; C07D 417/12; C07D 417/14

(52) U.S. Cl. 514/245; 514/252.05; 514/256; 544/212; 544/238; 544/333

(57) ABSTRACT

A compound of the formula (I) useful as promoters of thrombopoiesis and megakaryocytopoiesis, wherein A, B, D, E, W, X, Y, Z, R¹ and R² are defined as above.

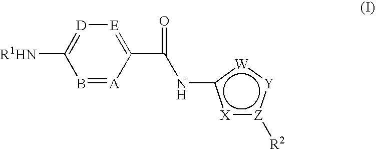

### FIELD OF THE INVENTION

This invention relates to thrombopoietin (TPO) mimetics and processes for the preparation of, intermediates used in the preparation of, compositions containing them and their use as promoters of thrombopoiesis and megakaryocytopoiesis.

### BACKGROUND OF THE INVENTION

Megakaryocytes are bone marrow-derived cells, which are responsible for producing circulating blood platelets. Although comprising <0.25% of the bone marrow cells in most species, they have >10 times the volume of typical marrow cells. See Kuter et al., Proc. Natl. Acad. Aci. USA, 91: 11104-11108 (1994). Megakaryocytes undergo a process known as endomitosis whereby they replicate their nuclei but fail to undergo cell division and thereby give rise to polyploid cells. In response to a decreased platelet count, the endomitotic rate increases, higher ploidy megakaryocytes are formed, and the number of megakaryocytes may increase up to 3-fold. See Harker, J. Clin. Invest. 47: 458-465 (1968). In contrast, in response to an elevated platelet count, the endomitotic rate decreases, lower ploidy megakaryocytes are formed, and the number of megakaryocytes may decrease by 50%.

The exact physiological feedback mechanism by which the mass of circulating platelets regulates the endomitotic rate and number of bone marrow megakaryocytes is not known. The circulating thrombopoietic factor involved in mediating this feedback loop is now thought to be thrombopoietin (TPO). More specifically, TPO has been shown to be the main humoral regulator in situations involving thrombocytopenia. See, e.g., Metcalf, Nature 369:519-520 (1994). TPO has been shown in several studies to increase platelet counts, increase platelet size, and increase isotope incorporation into platelets of recipient animals. Specifically, TPO is thought to affect megakaryocytopoiesis in several ways: (1) it produces increases in megakaryocyte size and number; (2) it produces an increase in DNA content, in the form of polyploidy, in megakaryocytes; (3) it increases megakaryocyte endomitosis; (4) it produces increased maturation of megakaryocytes; and (5) it produces an increase in the percentage of precursor cells, in the form of small acetylcholinesterase-positive cells, in the bone marrow.

Because platelets (thrombocytes) are necessary for blood clotting and when their numbers are very low a patient is at risk of death from catastrophic hemorrhage, TPO has potential useful application in both the diagnosis and the treatment of various hematological disorders, for example, diseases primarily due to platelet defects (See Harker et al., Blood 91: 4427-4433 (1998)). Ongoing clinical trials with TPO have indicated that TPO can be administered safely to patients (See Basser et al., Blood 89: 3118-3128 (1997); Fanucchi et al., New Engl. S. Med. 336: 404-409 (1997)). In addition, recent studies have provided a basis for the projection of efficacy of TPO therapy in the treatment of thrombocytopenia, and particularly thrombocytopenia resulting from chemotherapy, radiation therapy, or bone marrow transplantation as treatment for cancer or lymphoma. (See Harker, Curr. Opin. Hematol. 6: 127-134 (1999)).

The gene encoding TPO has been cloned and characterized. See Kuter et al., Proc. Natl. Acad. Sci. USA 91: 11104-11108 (1994); Barley et al., Cell 72:1117-1124 (1994); Kaushansky et al., Nature 369:568-571 (1994); Wending et al., Nature 369: 571-574 (1994); and Sauvage et al., Nature 369: 533-538 (1994).

Thrombopoietin is a glycoprotein with at least two forms, with apparent molecular masses of 251 kDa and 31 kDa, with a common N-terminal amino acid sequence. See Baatout, Haemostasis 27: 1-8 (1997); Kaushansky, New Engl. J. Med. 339: 746-754 (1998). Thrombopoietin appears to have two distinct regions separated by a potential Arg-Arg cleavage site. The amino-terminal region is highly conserved in man and mouse, and has some homology with erythropoietin and interferon-a and interferon-b. The carboxy-terminal region shows wide species divergence.

The DNA sequences and encoded peptide sequences for human TPO receptor (TPO—R; also known as c-mpl) have been described. (See, Vigon et al., Proc. Natl. Acad. Sci. USA 89: 5640-5644 (1992)). TPO—R is a member of the haematopoietin growth factor receptor family, a family characterized by a common structural design of the extracellular domain, including for conserved C residues in the N-terminal portion and a WSXWS motif close to the transmembrane region. (See Bazan, Proc. Natl. Acad. Sci. USA 87: 6934-6938 (1990)). Evidence that this receptor plays a functional role in hematopoiesis includes observations that its expression is restricted to spleen, bone marrow, or fetal liver in mice (See Souyri et al., Cell 63: 1137-1147 (1990)) and to megakaryocytes, platelets, and CD34⁺ cells in humans (See Methia et al., Blood 82: 1395-1401 (1993)). Further evidence for TPO—R as a key regulator of megakaryopoiesis is the fact that exposure of CD34⁺ cells to synthetic oligonucleotides antisense to TPO—R RNA significantly inhibits the appearance of megakaryocyte colonies without affecting erythroid or myeloid colony formation. Some workers postulate that the receptor functions as a homodimer, similar to the situation with the receptors for G-CSF and erythropoietin. (See Alexander et al., EMBO J. 14: 5569-5578 (1995)).

The slow recovery of platelet levels in patients suffering from thrombocytopenia is a serious problem, and has lent urgency to the search for a blood growth factor agonist able to accelerate platelet regeneration (See Kuter, Seminars in Hematology 37: Supp 4: 41-49 (2000)).

It would be desirable to provide compounds which allow for the treatment of thrombocytopenia by acting as a TPO mimetic.

As disclosed herein it has unexpectedly been discovered that certain benzimidazoles are effective as agonists of the TPO receptor and are potent TPO mimetics.

### SUMMARY OF THE INVENTION

The present invention relates to a compound of the Formula

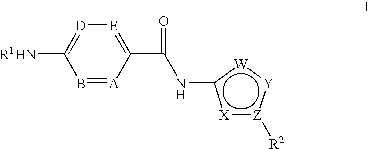

or the pharmaceutically acceptable salts thereof; wherein

R¹ is (C₂-C₈)heteroaryl or (C₂-C₉)heterocycloalkyl wherein the heteroaryl or heterocycloalkyl groups are optionally substituted by one to three groups selected from the group consisting of halo, cyano, nitro, carboxy, hydroxy, amino, NH₂C(O)—, R³(C₁-C₆)alkyl, R³(C₁-C₆)alkoxy, R³(C₁-C₆)alkoxycarbonyl, R³(C₁-C₆)alkylthio, R³(C₁-C₆)alkylsulfinyl, R³(C₁-C₆)alkylsulfonyl, R³(C₁-C₆)alkylaminosulfonyl, R³(C₁-C₆)alkylsulfonylamino, R³(C₁-C₆)alkylamino, R³(C₁-C₆)alkylcarboxy, R³(C₁-C₆)alkyl-NH—C(O)—, amino-C(O)—NH—, R³(C₁-C₆)alkylamino C(O)—NH—, aminocarbonyl(C₁-C₆)alkyl, R³(C₁-C₆)alkylaminocarbonyl(C₁-C₆)alkyl, amino-C(O)—O—, amino(C₁-C₆)alkoxycarbonyl, R³(C₁-C₆)alkylamino-C(O)—O—, R³(C₁-C₆)alkylamino(C₁-C₆)alkoxycarbonyl, R³(C₁-C₆)alkoxy-C(O)—NH—, R³(C₁-C₆)alkoxy(C₁-C₆)alkylamino, trifluoromethyl, trifluoromethyl(C₁-C₆)alkyl, R³(C₁-C₆)alkyl-CF₂, trifluoromethyl[(C₁-C₆)alkyl]ₐ-(CF₂)_{b}—[(C₁-C₆)alkyl]_{c}- wherein a is 0 or 1, b is 1, 2, 3 or 4, and c is 0 or 1; R⁴R⁵N—, R⁴R⁵N—C(O)—, R⁴R⁵N—C(O)—NH—, R⁴R⁵N—C(O)—(C₁-C₆)alkyl and R⁴R⁵N—C(O)—O—;

wherein R³ is one to three groups selected from hydrogen, (C₁-C₆)alkoxy, hydroxy, carboxy, amino, (C₁-C₆)alkylamino, R⁴R⁵N—, (C₁-C₆)alkylamino, (C₁-C₆)alkylthio, (C₁-C₆)alkylsulfinyl, (C₁-C₆)alkylsulfonyl and NH₂—C(O)—;

R⁴ and R⁵ are each independently (C₁-C₆)alkyl optionally substituted by (C₁-C₆)alkoxy, hydroxy, carboxy, amino, (C₁-C₆)alkylamino, amino, (C₁-C₆)alkylamino, ((C₁-C₆)alkyl)₂amino, (C₁-C₆)alkylthio, (C₁-C₆)alkylsulfinyl, (C₁-C₆)alkylsulfonyl and NH₂—C(O)—;

or R⁴ and R⁵ may be taken together with the nitrogen to which they are attached to form a 4 to 8 membered ring wherein the 6 to 8 membered rings may further optionally contain one to three heteroatoms selected from the group consisting of O, S, S(O), S(O)₂, NH or ((C₁-C₆)alkyl)-N—;

A, B, D, E are each independently CH, N or CR⁶ wherein R⁶ is halo, cyano, nitro, carboxy, hydroxy, amino, NH₂C(O)—, R⁷(C₁-C₆)alkyl, R⁷(C₁-C₆)alkoxy, R⁷(C₁-C₆)alkoxycarbonyl, R⁷(C₁-C₆)alkylthio, R⁷(C₁-C₆)alkylsulfinyl, R⁷(C₁-C₆)alkylsulfonyl, R⁷(C₁-C₆)alkylaminosulfonyl, R⁷(C₁-C₆)alkylsulfonylamino, R⁷(C₁-C₆)alkylamino, R⁷(C₁-C₆)alkylcarboxy, R⁷(C₁-C₆)alkyl-NH—C(O)—, amino-C(O)—NH—, R⁷(C₁-C₆)alkylamino C(O)—NH—, aminocarbonyl(C₁-C₆)alkyl, R⁷(C₁-C₆)alkylaminocarbonyl(C₁-C₆)alkyl, amino-C(O)—O—, amino(C₁-C₆)alkoxycarbonyl, R⁷(C₁-C₆)alkylamino-C(O)—O—, R⁷(C₁-C₆)alkylamino(C₁-C₆)alkoxycarbonyl, R⁷(C₁-C₆)alkoxy-C(O)—NH—, R⁷(C₁-C₆)alkoxy(C₁-C₆)alkylamino, trifluoromethyl, trifluoromethyl(C₁-C₆)alkyl, R⁷(C₁-C₆)alkyl-CF₂, trifluoromethyl[(C₁-C₆)alkyl]ₐ—(CF₂)_{b}-[(C₁-C₆)alkyl]_{c}- wherein a is 0 or 1, b is 1, 2, 3 or 4, and c is 0 or 1; R⁸R⁹N—, R⁸R⁹N—C(O)—, R⁸R⁹N—C(O)—NH—, R⁸R⁹N—C(O)—(C₁-C₆)alkyl and R⁸R⁹N—C(O)—O—;

wherein R⁷ is one to three groups selected from hydrogen, (C₁-C₆)alkoxy, hydroxy, carboxy, amino, (C₁-C₆)alkylamino, R⁸R⁹N—, (C₁-C₆)alkylamino, (C₁-C₆)alkylthio, (C₁-C₆)alkylsulfinyl, (C₁-C₆)alkylsulfonyl or NH₂—C(O)—;

R⁸ and R⁹ are each independently (C₁-C₆)alkyl optionally substituted by (C₁-C₆)alkoxy, hydroxy, carboxy, amino, (C₁-C₆)alkylamino, amino, (C₁-C₆)alkylamino, ((C₁-C₆)alkyl)₂-amino, (C₁-C₆)alkylthio, (C₁-C₆)alkylsulfinyl, (C₁-C₆)alkylsulfonyl and NH₂—C(O)—;

or R⁸ and R⁹ may be taken together with the nitrogen to which they are attached to form a 4 to 8 membered ring wherein the 6 to 8 membered rings may further optionally contain one to three heteroatoms selected from the group consisting of O, S, S(O), S(O)₂, NH or ((C₁-C₆)alkyl)-N—;

W, X and Y are each independently selected from the group consisting of C, CH, CR¹⁰, O, S, N, NH and R¹⁰((C₁-C₆)alkyl)-N;

Z is C or N;

wherein R¹⁰ is halo, cyano, nitro, carboxy, hydroxy, amino, NH₂C(O)—, R¹¹(C₁-C₆)alkyl, R¹¹(C₁-C₆)alkoxy, R¹¹(C₁-C₆)alkoxycarbonyl, R¹¹(C₁-C₆)alkylthio, R¹¹(C₁-C₆)alkylsulfinyl, R¹¹(C₁-C₆)alkylsulfonyl, R¹¹(C₁-C₆)alkylaminosulfonyl, R¹¹(C₁-C₆)alkylsulfonylamino, R¹¹(C₁-C₆)alkylamino, R¹¹(C₁-C₆)alkylcarboxy, R¹¹(C₁-C₆)alkyl-NH—C(O)—, amino-C(O)—NH—, R¹¹(C₁-C₆)alkylamino C(O)—NH—, aminocarbonyl(C₁-C₆)alkyl, R¹¹(C₁-C₆)alkylaminocarbonyl(C₁-C₆)alkyl, amino-C(O)—O—, amino(C₁-C₆)alkoxycarbonyl, R(C₁-C₆)alkylamino-C(O)—O—, R(C₁-C₆)alkylamino(C₁-C₆)alkoxycarbonyl, R(C₁-C₆)alkoxy-C(O)—NH—, R¹¹ (C₁-C₆)alkoxy(C₁-C₆)alkylamino, trifluoromethyl, trifluoromethyl(C₁-C₆)alkyl, R¹¹(C₁-C₆)alkyl-CF₂, trifluoromethyl[(C₁-C₆)alkyl]ₐ—(CF₂)_{b}-[(C₁-C₆)alkyl]_{c}-, wherein a is 0 or 1, b is 1, 2, 3 or 4, and c is 0 or 1; R¹²R¹³N—, R¹²R¹³N—C(O)—, R¹²R¹³N—C(O)—NH—, R¹²R¹³N—C(O)—(C₁-C₆)alkyl and R¹²R¹³N—C(O)—O—;

wherein R¹¹ is one to three groups selected from hydrogen, (C₁-C₆)alkoxy, hydroxy, carboxy, amino, (C₁-C₆)alkylamino, R¹²R¹³N—, (C₁-C₆)alkylamino, (C₁-C₆)alkylthio, (C₁-C₆)alkylsulfinyl, (C₁-C₆)alkylsulfonyl and NH₂—C(O)—;

R¹² and R¹³ are each independently (C₁-C₆)alkyl optionally substituted by (C₁-C₆)alkoxy, hydroxy, carboxy, amino, (C₁-C₆)alkylamino, amino, (C₁-C₆)alkylamino, ((C₁-C₆)alkyl)₂amino, (C₁-C₆)alkylthio, (C₁-C₆)alkylsulfinyl, (C₁-C₆)alkylsulfonyl and NH₂—C(O)—;

or R¹² and R¹³ may be taken together with the nitrogen to which they are attached to form a 4 to 8 membered ring wherein the 6 to 8 membered rings may further optionally contain one to three heteroatoms selected from the group consisting of O, S, S(O), S(O)₂, NH or ((C₁-C₆)alkyl)-N—; and

R² is R¹⁴(C₆-C₁₀)aryl, R¹⁴(C₂-C₉)heteroaryl, R¹⁴(C₃-C₁₀)cycloalkyl or R¹⁴(C₂-C₉)heterocycloalkyl; wherein R¹⁴ is one to three groups selected from hydrogen, halo, cyano, nitro, carboxy, hydroxy, amino, NH₂C(O)—, R¹⁵(C₁-C₆)alkyl, R¹⁵(C₁-C₆)alkoxy, R¹⁵(C₁-C₆)alkoxycarbonyl, R¹⁵(C₁-C₆)alkylthio, R¹⁵(C₁-C₆)alkylsulfinyl, R¹⁵(C₁-C₆)alkylsulfonyl, R¹⁵(C₁-C₆)alkylaminosulfonyl, R¹⁵(C₁-C₆)alkylsulfonylamino, R¹⁵(C₁-C₆)alkylamino, R¹⁵(C₁-C₆)alkylcarboxy, R¹⁵(C₁-C₆)alkyl-NH—C(O)—, amino-C(O)—NH—, R¹⁵(C₁-C₆)alkylamino C(O)—NH—, aminocarbonyl(C₁-C₆)alkyl, R¹⁵(C₁-C₆)alkylaminocarbonyl(C₁-C₆)alkyl, amino-C(O)—O—, amino(C₁-C₆)alkoxycarbonyl, R¹⁵(C₁-C₆)alkylamino-C(O)—O—, R¹⁵(C₁-C₆)alkylamino(C₁-C₆)alkoxycarbonyl, R¹⁵(C₁-C₆)alkoxy-C(O)—NH—, R¹⁵(C₁-C₆)alkoxy(C₁-C₆)alkylamino, trifluoromethyl, trifluoromethyl(C₁-C₆)alkyl, R¹⁵(C₁-C₆)alkyl-CF₂, trifluoromethyl[(C₁-C₆)alkyl]ₐ-(CF₂)_{b}-[(C₁-C₆)alkyl]_{c}-, wherein a is 0 or 1, b is 1, 2, 3 or 4, and c is 0 or 1; R¹⁶R¹⁷N—, R¹⁶R¹⁷N—C(O)—, R¹⁶R¹⁷N—C(O)—NH—, R¹⁶R¹⁷N—C(O)—(C₁-C₆)alkyl and R¹⁶R¹⁷N—C(O)—O—;

wherein R¹⁵ is one to three groups selected from hydrogen, (C₁-C₆)alkoxy, hydroxy, carboxy, amino, (C₁-C₆)alkylamino, R¹⁶R¹⁷N—, (C₁-C₆)alkylamino, (C₁-C₆)alkylthio, (C₁-C₆)alkylsulfinyl, (C₁-C₆)alkylsulfonyl and NH₂—C(O)—;

R¹⁶ and R¹⁷ are each independently (C₁-C₆)alkyl optionally substituted by (C₁-C₆)alkoxy, hydroxy, carboxy, amino, (C₁-C₆)alkylamino, amino, (C₁-C₆)alkylamino, ((C₁-C₆)alkyl)₂amino, (C₁-C₆)alkylthio, (C₁-C₆)alkylsulfinyl, (C₁-C₆)alkylsulfonyl and NH₂—C(O)—;

or R¹⁶ and R¹⁷ may be taken together with the nitrogen to which they are attached to form a 4 to 8 membered ring wherein the 6 to 8 membered rings may further optionally contain one to three heteroatoms selected from the group consisting of O, S, S(O), S(O)₂, NH or ((C₁-C₆)alkyl)-N—.

The present invention further relates to a compound of formula I wherein R¹ is (C₂-C₉)heteroaryl.

The present invention further relates to a compound of formula I wherein R¹ is pyridine, pyrazine, pyrimidine, pyridazine, 1,3,5-triazine, 1,2,4-triazine and 1,2,3-triazine.

The present invention further relates to a compound of formula I wherein R² is phenyl, pyridinyl, pyrimidinyl, pyrazinyl and pyridazinyl.

The present invention further relates to a compound of formula I wherein X is N or CH, W is O, S or NH; Y is N or CH; and Z is C.

The present invention further relates to a compound of formula I wherein X is O, S or NH; W is N or CH; Y is N or CH and Z is C.

The present invention further relates to a compound of formula I wherein X is N or CH, W is N or CH, Y is O, S or NH and Z is C.

The present invention further relates to a compound of formula I wherein X is N or CH; W is N or CH; Y is N or CH and Z is N.

The present invention further relates to a compound of formula I wherein X is N; W is S; Y is CH and Z is C.

The present invention further relates to a compound of formula I wherein X is N; W is S; Y is N and Z is C.

The present invention further relates to a compound of formula I wherein A is N; B is CH; D is CH and E is CH.

The present invention further relates to a compound of formula I wherein A is CH; B is N; D is CH and E is CH.

The present invention further relates to a compound of formula I wherein A is N; B is CH; D is CH and E is N.

The present invention further relates to a compound of formula I wherein A is N; B is CH; D is N and E is CH.

The present invention further relates to a compound of formula I wherein A is N; B is N; D is CH and E is CH.

The present invention further relates to a compound of formula I wherein A is N; B is N; D is N and E is CH.

The present invention further relates to a compound of formula I, wherein A is N; B is CH; D is N and E is N.

The present invention further relates to a compound of formula I wherein A is CH; B is CH; D is CH and E is CH.

The present invention further relates to a compound of formula I wherein R¹ is pyridine, pyrazine, pyrimidine, pyridazine, 1,3,5-triazine, 1,2,4-triazine and 1,2,3-triazine; R² is phenyl, pyridinyl, pyrimidinyl, pyrazinyl and pyridazinyl; X is N or CH, W is O, S or NH; Y is N or CH; Z is C; A is N; B is CH; D is CH and E is CH.

The present invention further relates to a compound of formula I wherein R¹ is pyridine, pyrazine, pyrimidine, pyridazine, 1,3,5-triazine, 1,2,4-triazine and 1,2,3-triazine; R² is phenyl, pyridinyl, pyrimidinyl, pyrazinyl and pyridazinyl; X is O, S or NH; W is N or CH; Y is N or CH; Z is C A is N; B is CH; D is CH and E is CH.

The present invention further relates to a compound of formula I wherein R¹ is pyridine, pyrazine, pyrimidine, pyridazine, 1,3,5-triazine, 1,2,4-triazine and 1,2,3-triazine; R² is phenyl, pyridinyl, pyrimidinyl, pyrazinyl and pyridazinyl; X is N or CH, W is N or CH, Y is O, S or NH; Z is C; A is N; B is CH; D is CH and E is CH.

The present invention further relates to a compound of formula I wherein R¹ is pyridine, pyrazine, pyrimidine, pyridazine, 1,3,5-triazine, 1,2,4-triazine and 1,2,3-triazine; R² is phenyl, pyridinyl, pyrimidinyl, pyrazinyl and pyridazinyl; X is N or CH; W is N or CH; Y is N or CH; Z is N; A is N; B is CH; D is CH and E is CH.

The present invention further relates to a compound of formula I wherein R¹ is pyridine, pyrazine, pyrimidine, pyridazine, 1,3,5-triazine, 1,2,4-triazine and 1,2,3-triazine; R² is phenyl, pyridinyl, pyrimidinyl, pyrazinyl and pyridazinyl; X is N; W is S; Y is CH; Z is C; A is N; B is CH; D is CH and E is CH.

The present invention further relates to a compound of formula I wherein R¹ is pyridine, pyrazine, pyrimidine, pyridazine, 1,3,5-triazine, 1,2,4-triazine and 1,2,3-triazine; R² is phenyl, pyridinyl, pyrimidinyl, pyrazinyl and pyridazinyl; X is N; W is S; Y is N; Z is C; A is N; B is CH; D is CH and E is CH.

The present invention further relates to a compound of formula I wherein R¹ is pyridine, pyrazine, pyrimidine, pyridazine, 1,3,5-triazine, 1,2,4-triazine and 1,2,3-triazine; R² is phenyl, pyridinyl, pyrimidinyl, pyrazinyl and pyridazinyl; X is N or CH, W is O, S or NH; Y is N or CH; Z is C; A is CH; B is N; D is CH and E is CH.

The present invention further relates to a compound of formula I wherein R¹ is pyridine, pyrazine, pyrimidine, pyridazine, 1,3,5-triazine, 1,2,4-triazine and 1,2,3-triazine; R² is phenyl, pyridinyl, pyrimidinyl, pyrazinyl and pyridazinyl; X is O, S or NH; W is N or CH; Y is N or CH; Z is C; A is CH; B is N; D is CH and E is CH.

The present invention further relates to a compound of formula I wherein R¹ is pyridine, pyrazine, pyrimidine, pyridazine, 1,3,5-triazine, 1,2,4-triazine and 1,2,3-triazine; R² is phenyl, pyridinyl, pyrimidinyl, pyrazinyl and pyridazinyl; X is N or CH, W is N or CH, Y is O, S or NH; Z is C; A is CH; B is N; D is CH and E is CH.

The present invention further relates to a compound of formula I wherein R¹ is pyridine, pyrazine, pyrimidine, pyridazine, 1,3,5-triazine, 1,2,4-triazine and 1,2,3-triazine; R² is phenyl, pyridinyl, pyrimidinyl, pyrazinyl and pyridazinyl; X is N or CH; W is N or CH; Y is N or CH; Z is N; A is CH; B is N; D is CH and E is CH.

The present invention further relates to a compound of formula I wherein R¹ is pyridine, pyrazine, pyrimidine, pyridazine, 1,3,5-triazine, 1,2,4-triazine and 1,2,3-triazine; R² is phenyl, pyridinyl, pyrimidinyl, pyrazinyl and pyridazinyl; X is N; W is S; Y is CH; Z is C; A is CH; B is N; D is CH and E is CH.

The present invention further relates to a compound of formula I wherein R¹ is pyridine, pyrazine, pyrimidine, pyridazine, 1,3,5-triazine, 1,2,4-triazine and 1,2,3-triazine; R² is phenyl, pyridinyl, pyrimidinyl, pyrazinyl and pyridazinyl; X is N; W is S; Y is N; Z is C; A is CH; B is N; D is CH and E is CH.

The present invention further relates to a compound of formula I wherein R¹ is pyridine, pyrazine, pyrimidine, pyridazine, 1,3,5-triazine, 1,2,4-triazine and 1,2,3-triazine; R² is phenyl, pyridinyl, pyrimidinyl, pyrazinyl and pyridazinyl; X is N or CH, W is O, S or NH; Y is N or CH; Z is C; A is N; B is CH; D is CH and E is N.

The present invention further relates to a compound of formula I wherein R¹ is pyridine, pyrazine, pyrimidine, pyridazine, 1,3,5-triazine, 1,2,4-triazine and 1,2,3-triazine; R² is phenyl, pyridinyl, pyrimidinyl, pyrazinyl and pyridazinyl; X is O, S or NH; W is N or CH; Y is N or CH; Z is C; A is N; B is CH; D is CH and E is N.

The present invention further relates to a compound of formula I wherein R¹ is pyridine, pyrazine, pyrimidine, pyridazine, 1,3,5-triazine, 1,2,4-triazine and 1,2,3-triazine; R² is phenyl, pyridinyl, pyrimidinyl, pyrazinyl and pyridazinyl; X is N or CH, W is N or CH, Y is O, S or NH; Z is C; A is N; B is CH; D is CH and E is N.

The present invention further relates to a compound of formula I wherein R¹ is pyridine, pyrazine, pyrimidine, pyridazine, 1,3,5-triazine, 1,2,4-triazine and 1,2,3-triazine; R² is phenyl, pyridinyl, pyrimidinyl, pyrazinyl and pyridazinyl; X is N or CH; W is N or CH; Y is N or CH; Z is N; A is N; B is CH; D is CH and E is N.

The present invention further relates to a compound of formula I wherein R¹ is pyridine, pyrazine, pyrimidine, pyridazine, 1,3,5-triazine, 1,2,4-triazine and 1,2,3-triazine; R² is phenyl, pyridinyl, pyrimidinyl, pyrazinyl and pyridazinyl; X is N; W is S; Y is CH; Z is C; A is N; B is CH; D is CH and E is N.

The present invention further relates to a compound of formula I wherein R¹ is pyridine, pyrazine, pyrimidine, pyridazine, 1,3,5-triazine, 1,2,4-triazine and 1,2,3-triazine; R² is phenyl, pyridinyl, pyrimidinyl, pyrazinyl and pyridazinyl; X is N; W is S; Y is N; Z is C; A is N; B is CH; D is CH and E is N.

The present invention further relates to a compound of formula I wherein R¹ is pyridine, pyrazine, pyrimidine, pyridazine, 1,3,5-triazine, 1,2,4-triazine and 1,2,3-triazine; R² is phenyl, pyridinyl, pyrimidinyl, pyrazinyl and pyridazinyl; X is N or CH, W is O, S or NH; Y is N or CH; Z is C; A is N; B is CH; D is N and E is CH.

The present invention further relates to a compound of formula I wherein R¹ is pyridine, pyrazine, pyrimidine, pyridazine, 1,3,5-triazine, 1,2,4-triazine and 1,2,3-triazine; R² is phenyl, pyridinyl, pyrimidinyl, pyrazinyl and pyridazinyl; X is O, S or NH; W is N or CH; Y is N or CH; Z is C; A is N; B is CH; D is N and E is CH.

The present invention further relates to a compound of formula I wherein R¹ is pyridine, pyrazine, pyrimidine, pyridazine, 1,3,5-triazine, 1,2,4-triazine and 1,2,3-triazine; R² is phenyl, pyridinyl, pyrimidinyl, pyrazinyl and pyridazinyl; X is N or CH, W is N or CH, Y is O, S or NH; Z is C; A is N; B is CH; D is N and E is CH.

The present invention further relates to a compound of formula I wherein R¹ is pyridine, pyrazine, pyrimidine, pyridazine, 1,3,5-triazine, 1,2,4-triazine and 1,2,3-triazine; R² is phenyl, pyridinyl, pyrimidinyl, pyrazinyl and pyridazinyl; X is N or CH; W is N or CH; Y is N or CH; Z is N; A is N; B is CH; D is N and E is CH.

The present invention further relates to a compound of formula I wherein R¹ is pyridine, pyrazine, pyrimidine, pyridazine, 1,3,5-triazine, 1,2,4-triazine and 1,2,3-triazine; R² is phenyl, pyridinyl, pyrimidinyl, pyrazinyl and pyridazinyl; X is N; W is S; Y is CH; Z is C; A is N; B is CH; D is N and E is CH.

The present invention further relates to a compound of formula I wherein R¹ is pyridine, pyrazine, pyrimidine, pyridazine, 1,3,5-triazine, 1,2,4-triazine and 1,2,3-triazine; R² is phenyl, pyridinyl, pyrimidinyl, pyrazinyl and pyridazinyl; X is N; W is S; Y is N; Z is C; A is N; B is CH; D is N and E is CH.

The present invention further relates to a compound of formula I wherein R¹ is pyridine, pyrazine, pyrimidine, pyridazine, 1,3,5-triazine, 1,2,4-triazine and 1,2,3-triazine; R² is phenyl, pyridinyl, pyrimidinyl, pyrazinyl and pyridazinyl; X is N or CH, W is O, S or NH; Y is N or CH; Z is C; A is N; B is N; D is CH and E is CH.

The present invention further relates to a compound of formula I wherein R¹ is pyridine, pyrazine, pyrimidine, pyridazine, 1,3,5-triazine, 1,2,4-triazine and 1,2,3-triazine; R² is phenyl, pyridinyl, pyrimidinyl, pyrazinyl and pyridazinyl; X is O, S or NH; W is N or CH; Y is N or CH; Z is C; A is N; B is N; D is CH and E is CH.

The present invention further relates to a compound of formula I wherein R¹ is pyridine, pyrazine, pyrimidine, pyridazine, 1,3,5-triazine, 1,2,4-triazine and 1,2,3-triazine; R² is phenyl, pyridinyl, pyrimidinyl, pyrazinyl and pyridazinyl; X is N or CH, W is N or CH, Y is O, S or NH; Z is C; A is N; B is N; D is CH and E is CH.

The present invention further relates to a compound of formula I wherein R¹ is pyridine, pyrazine, pyrimidine, pyridazine, 1,3,5-triazine, 1,2,4-triazine and 1,2,3-triazine; R² is phenyl, pyridinyl, pyrimidinyl, pyrazinyl and pyridazinyl; X is N or CH; W is N or CH; Y is N or CH; Z is N; A is N; B is N; D is CH and E is CH.

The present invention further relates to a compound of formula I wherein R¹ is pyridine, pyrazine, pyrimidine, pyridazine, 1,3,5-triazine, 1,2,4-triazine and 1,2,3-triazine; R² is phenyl, pyridinyl, pyrimidinyl, pyrazinyl and pyridazinyl; X is N; W is S; Y is CH; Z is C A is N; B is N; D is CH and E is CH.

The present invention further relates to a compound of formula I wherein R¹ is pyridine, pyrazine, pyrimidine, pyridazine, 1,3,5-triazine, 1,2,4-triazine and 1,2,3-triazine; R² is phenyl, pyridinyl, pyrimidinyl, pyrazinyl and pyridazinyl; X is N; W is S; Y is N; Z is C; A is N; B is N; D is CH and E is CH.

The present invention further relates to a compound of formula I wherein R¹ is pyridine, pyrazine, pyrimidine, pyridazine, 1,3,5-triazine, 1,2,4-triazine and 1,2,3-triazine; R² is phenyl, pyridinyl, pyrimidinyl, pyrazinyl and pyridazinyl; X is N or CH, W is O, S or NH; Y is N or CH; Z is C; A is N; B is N; D is N and E is CH.

The present invention further relates to a compound of formula I wherein R¹ is pyridine, pyrazine, pyrimidine, pyridazine, 1,3,5-triazine, 1,2,4-triazine and 1,2,3-triazine; R² is phenyl, pyridinyl, pyrimidinyl, pyrazinyl and pyridazinyl; X is O, S or NH; W is N or CH; Y is N or CH; Z is C A is N; B is N; D is N and E is CH.

The present invention further relates to a compound of formula I wherein R¹ is pyridine, pyrazine, pyrimidine, pyridazine, 1,3,5-triazine, 1,2,4-triazine and 1,2,3-triazine; R² is phenyl, pyridinyl, pyrimidinyl, pyrazinyl and pyridazinyl; X is N or CH, W is N or CH, Y is O, S or NH; Z is C; A is N; B is N; D is N and E is CH.

The present invention further relates to a compound of formula I wherein R¹ is pyridine, pyrazine, pyrimidine, pyridazine, 1,3,5-triazine, 1,2,4-triazine and 1,2,3-triazine; R² is phenyl, pyridinyl, pyrimidinyl, pyrazinyl and pyridazinyl; X is N or CH; W is N or CH; Y is N or CH; Z is N; A is N; B is N; D is N and E is CH.

The present invention further relates to a compound of formula I wherein R¹ is pyridine, pyrazine, pyrimidine, pyridazine, 1,3,5-triazine, 1,2,4-triazine and 1,2,3-triazine; R² is phenyl, pyridinyl, pyrimidinyl, pyrazinyl and pyridazinyl; X is N; W is S; Y is CH; Z is C; A is N; B is N; D is N and E is CH.

The present invention further relates to a compound of formula I wherein R¹ is pyridine, pyrazine, pyrimidine, pyridazine, 1,3,5-triazine, 1,2,4-triazine and 1,2,3-triazine; R² is phenyl, pyridinyl, pyrimidinyl, pyrazinyl and pyridazinyl; X is N; W is S; Y is N; Z is C; A is N; B is N; D is N and E is CH.

The present invention further relates to a compound of formula I wherein R¹ is pyridine, pyrazine, pyrimidine, pyridazine, 1,3,5-triazine, 1,2,4-triazine and 1,2,3-triazine; R² is phenyl, pyridinyl, pyrimidinyl, pyrazinyl and pyridazinyl; X is N or CH, W is O, S or NH; Y is N or CH; Z is C; A is N; B is CH; D is N and E is N.

The present invention further relates to a compound of formula I wherein R¹ is pyridine, pyrazine, pyrimidine, pyridazine, 1,3,5-triazine, 1,2,4-triazine and 1,2,3-triazine; R² is phenyl, pyridinyl, pyrimidinyl, pyrazinyl and pyridazinyl; X is O, S or NH; W is N or CH; Y is N or CH; Z is C; A is N; B is CH; D is N and E is N.

The present invention further relates to a compound of formula I wherein R¹ is pyridine, pyrazine, pyrimidine, pyridazine, 1,3,5-triazine, 1,2,4-triazine and 1,2,3-triazine; R² is phenyl, pyridinyl, pyrimidinyl, pyrazinyl and pyridazinyl; X is N or CH, W is N or CH, Y is O, S or NH; Z is C; A is N; B is CH; D is N and E is N.

The present invention further relates to a compound of formula I wherein R¹ is pyridine, pyrazine, pyrimidine, pyridazine, 1,3,5-triazine, 1,2,4-triazine and 1,2,3-triazine; R² is phenyl, pyridinyl, pyrimidinyl, pyrazinyl and pyridazinyl; X is N or CH; W is N or CH; Y is N or CH; Z is N; A is N; B is CH; D is N and E is N.

The present invention further relates to a compound of formula I wherein R¹ is pyridine, pyrazine, pyrimidine, pyridazine, 1,3,5-triazine, 1,2,4-triazine and 1,2,3-triazine; R² is phenyl, pyridinyl, pyrimidinyl, pyrazinyl and pyridazinyl; X is N; W is S; Y is CH; Z is C; A is N; B is CH; D is N and E is N.

The present invention further relates to a compound of formula I wherein R¹ is pyridine, pyrazine, pyrimidine, pyridazine, 1,3,5-triazine, 1,2,4-triazine and 1,2,3-triazine; R² is phenyl, pyridinyl, pyrimidinyl, pyrazinyl and pyridazinyl; X is N; W is S; Y is N; Z is C; A is N; B is CH; D is N and E is N.

The present invention further relates to a compound of formula I wherein R¹ is pyridine, pyrazine, pyrimidine, pyridazine, 1,3,5-triazine, 1,2,4-triazine and 1,2,3-triazine; R² is phenyl, pyridinyl, pyrimidinyl, pyrazinyl and pyridazinyl; X is N or CH, W is O, S or NH; Y is N or CH; Z is C; A is CH; B is CH; D is CH and E is CH.

The present invention further relates to a compound of formula I wherein R¹ is pyridine, pyrazine, pyrimidine, pyridazine, 1,3,5-triazine, 1,2,4-triazine and 1,2,3-triazine; R² is phenyl, pyridinyl, pyrimidinyl, pyrazinyl and pyridazinyl; X is O, S or NH; W is N or CH; Y is N or CH; Z is C; A is CH; B is CH; D is CH and E is CH.

The present invention further relates to a compound of formula I wherein R¹ is pyridine, pyrazine, pyrimidine, pyridazine, 1,3,5-triazine, 1,2,4-triazine and 1,2,3-triazine; R² is phenyl, pyridinyl, pyrimidinyl, pyrazinyl and pyridazinyl; X is N or CH, W is N or CH, Y is O, S or NH; Z is C; A is CH; B is CH; D is CH and E is CH.

The present invention further relates to a compound of formula I wherein R¹ is pyridine, pyrazine, pyrimidine, pyridazine, 1,3,5-triazine, 1,2,4-triazine and 1,2,3-triazine; R² is phenyl, pyridinyl, pyrimidinyl, pyrazinyl and pyridazinyl; X is N or CH; W is N or CH; Y is N or CH; Z is N; A is CH; B is CH; D is CH and E is CH.

The present invention further relates to a compound of formula I wherein R¹ is pyridine, pyrazine, pyrimidine, pyridazine, 1,3,5-triazine, 1,2,4-triazine and 1,2,3-triazine; R² is phenyl, pyridinyl, pyrimidinyl, pyrazinyl and pyridazinyl; X is N; W is S; Y is CH; Z is C; A is CH; B is CH; D is CH and E is CH.

The present invention further relates to a compound of formula I wherein R¹ is pyridine, pyrazine, pyrimidine, pyridazine, 1,3,5-triazine, 1,2,4-triazine and 1,2,3-triazine; R² is phenyl, pyridinyl, pyrimidinyl, pyrazinyl and pyridazinyl; X is N; W is S; Y is N; Z is C; A is CH; B is CH; D is CH and E is CH.

The present invention further relates to a compound selected from the group consisting of:
N-[2-(4-Fluoro-3-trifluoromethyl-phenyl)-oxazol-4-yl]-4-(pyrimidin-4-ylamino)-benzamide;
N-[2-(2-Fluoro-3-trifluoromethyl-phenyl)-oxazol-4-yl]-4-(pyrimidin-4-ylamino)-benzamide;
N-[2-(2,4-Difluoro-phenyl)-thiazol-4-yl]-4-(pyrimidin-4-ylamino)-benzamide;
N-[2-(2-Fluoro-3-trifluoromethyl-phenyl)-thiazol-4-yl]-4-(pyrimidin-4-ylamino)-benzamide;
N-[2-(4-Fluoro-3-trifluoromethyl-phenyl)-thiazol-4-yl]-4-(pyrimidin-4-ylamino)-benzamide;
N-[3-(4-Fluoro-3-trifluoromethyl-phenyl)-[1,2,4]thiadiazol-5-yl]-4-(pyrimidin-4-ylamino)-benzamide;
N-[3-(2,4-Difluoro-phenyl)-[1,2,4]thiadiazol-5-yl]-4-(pyrimidin-4-ylamino)-benzamide;
N-[3-(2-Fluoro-3-trifluoromethyl-phenyl)-[1,2,4]thiadiazol-5-yl]-4-(pyrimidin-4-ylamino)-benzamide;
N-(3-Phenyl-[1,2,4]thiadiazol-5-yl)-4-(pyrimidin-4-ylamino)-benzamide;
4-(6-Azetidin-1-yl-pyrimidin-4-ylamino)-N-[3-(4-fluoro-3-trifluoromethyl-phenyl)-[1,2,4]thiadiazol-5-yl]-benzamide;
4-(6-Dim ethylamino-pyrimidin-4-ylamino)-N-[3-(4-fluoro-3-trifluoromethyl-phenyl)-[1,2,4]thiadiazol-5-yl]-benzamide;
N-[3-(3-Trifluoromethoxy-phenyl)-[1,2,4]thiadiazol-5-yl]-4-(pyrimidin-4-ylamino)-benzamide;
N-[3-(3,4-Dichloro-phenyl)-[1,2,4]thiadiazol-5-yl]-4-(pyrimidin-4-ylamino)-benzamide;
N-[3-(3-Fluoro-4-trifluoromethyl-phenyl)-[1,2,4]thiadiazol-5-yl]-4-(pyrimidin-4-ylamino)-benzamide;
N-[3-(3,4-Difluoro-phenyl)-[1,2,4]thiadiazol-5-yl]-4-(pyrimidin-4-ylamino)-benzamide;
N-[5-(4-Fluoro-3-trifluoromethyl-phenyl)-[1,2,4]thiadiazol-3-yl]-4-(pyridazin-4-ylamino)-benzamide;
N-[5-(3,4-Difluoro-phenyl)-[1,2,4]thiadiazol-3-yl]-4-(pyrimidin-4-ylamino)-benzamide;
N-[4-(2-Fluoro-3-trifluoromethyl-phenyl)-thiazol-2-yl]-4-(pyrimidin-4-ylamino)-benzamide;
N-[4-(Phenyl)-thiazol-2-yl]-4-(pyrimidin-4-ylamino)-benzamide;
N-[4-(2-Fluoro-phenyl)-thiazol-2-yl]-4-(pyrimidin-4-ylamino)-benzamide;
N-[4-(3-Fluoro-phenyl)-thiazol-2-yl]-4-(pyrimidin-4-ylamino)-benzamide;
N-[4-(4-Fluoro-phenyl)-thiazol-2-yl]-4-(pyrimidin-4-ylamino)-benzamide;
N-[4-(2,3-Difluoro-phenyl)-thiazol-2-yl]-4-(pyrimidin-4-ylamino)-benzamide;
N-[4-(2,4-Difluoro-phenyl)-thiazol-2-yl]-4-(pyrimidin-4-ylamino)-benzamide;
N-[4-(2,6-Difluoro-phenyl)-thiazol-2-yl]-4-(pyrimidin-4-ylamino)-benzamide;
N-[4-(3,4-Difluoro-phenyl)-thiazol-2-yl]-4-(pyrimidin-4-ylamino)-benzamide;
N-[4-(2-Chloro-phenyl)-thiazol-2-yl]-4-(pyrimidin-4-ylamino)-benzamide;
N-[4-(3-Chloro-phenyl)-thiazol-2-yl]-4-(pyrimidin-4-ylamino)-benzamide;
N-[4-(4-Chloro-phenyl)-thiazol-2-yl]-4-(pyrimidin-4-ylamino)-benzamide;
N-[4-(2,3-Dichloro-phenyl)-thiazol-2-yl]-4-(pyrimidin-4-ylamino)-benzamide;
N-[4-(2,4-Dichloro-phenyl)-thiazol-2-yl]-4-(pyrimidin-4-ylamino)-benzamide;
N-[4-(3,4-Dichloro-phenyl)-thiazol-2-yl]-4-(pyrimidin-4-ylamino)-benzamide;
N-[4-(2-Trifluoromethyl-phenyl)-thiazol-2-yl]-4-(pyrimidin-4-ylamino)-benzamide;
N-[4-(3-Trifluoromethyl-phenyl)-thiazol-2-yl]-4-(pyrimidin-4-ylamino)-benzamide;
N-[4-(4-Trifluoromethyl-phenyl)-thiazol-2-yl]-4-(pyrimidin-4-ylamino)-benzamide;
N-[4-(2-Fluoro-3-chloro-phenyl)-thiazol-2-yl]-4-(pyrimidin-4-ylamino)-benzamide;
N-[4-(2-Fluoro-4-chloro-phenyl)-thiazol-2-yl]-4-(pyrimidin-4-ylamino)-benzamide;
N-[4-(2-Fluoro-4-trifluoromethyl-phenyl)-thiazol-2-yl]-4-(pyrimidin-4-ylamino)-benzamide;
N-[4-(2-Fluoro-5-trifluoromethyl-phenyl)-thiazol-2-yl]-4-(pyrimidin-4-ylamino)-benzamide;
N-[4-(4-Fluoro-3-trifluoromethyl-phenyl)-thiazol-2-yl]-4-(pyrimidin-4-ylamino)-benzamide;
N-[4-(3-Fluoro-4-trifluoromethyl-phenyl)-thiazol-2-yl]-4-(pyrimidin-4-ylamino)-benzamide;
N-[4-(2,6-Dichloro-4-trifluoromethyl-phenyl)-thiazol-2-yl]-4-(pyrimidin-4-ylamino)-benzamide;
N-[4-(4-Methyl-phenyl)-thiazol-2-yl]-4-(pyrimidin-4-ylamino)-benzamide;
N-[4-(2,4-Dimethyl-phenyl)-thiazol-2-yl]-4-(pyrimidin-4-ylamino)-benzamide;
N-[4-(4-Difluoromethoxy-phenyl)-thiazol-2-yl]-4-(pyrimidin-4-ylamino)-benzamide;
N-[4-(3-Trifluoromethoxy-phenyl)-thiazol-2-yl]-4-(pyrimidin-4-ylamino)-benzamide;
N-[4-(4-Trifluoromethoxy-phenyl)-thiazol-2-yl]-4-(pyrimidin-4-ylamino)-benzamide;
N-[4-(2-Naphthyl)-thiazol-2-yl]-4-(pyrimidin-4-ylamino)-benzamide;
4-(Pyrimidin-4-ylamino)-N-[4-(3-bromo-phenyl)-thiazol-2-yl]-benzamide;
4-(Pyrimidin-4-ylamino)-N-[4-(2-fluoro-3-bromo-phenyl)-thiazol-2-yl]-benzamide;
4-(Pyrimidin-4-ylamino)-N-[4-(2,3,4-trifluoro-phenyl)-thiazol-2-yl]-benzamide;
4-(Pyrimidin-4-ylamino)-N-[4-(2,3-difluoro-4-trifluoromethyl-phenyl)-thiazol-2-yl]-benzamide;
4-(Pyrimidin-4-ylamino)-N-[4-(6-trifluoromethyl-pyridin-2-yl)-thiazol-2-yl]-benzamide;
4-(Pyrimidin-4-ylamino)-N-[4-(2-fluoro-3-trifluoromethoxy-phenyl)-thiazol-2-yl]-benzamide;
4-(Pyridin-2-ylamino)-N-[4-(2-fluoro-3-trifluoromethyl-phenyl)-thiazol-2-yl]-benzamide;
4-(Pyridin-3-ylamino)-N-[4-(2-fluoro-3-trifluoromethyl-phenyl)-thiazol-2-yl]-benzamide;
4-(Pyridin-4-ylamino)-N-[4-(2-fluoro-3-trifluoromethyl-phenyl)-thiazol-2-yl]-benzamide;
4-(Pyridazin-4-ylamino)-N-[4-(2-fluoro-3-trifluoromethyl-phenyl)-thiazol-2-yl]-benzamide;
4-(Pyridazin-4-ylamino)-N-[4-(4-chloro-phenyl)-thiazol-2-yl]-benzamide;
4-(Pyridazin-4-ylamino)-N-[4-(2,4-difluoro-phenyl)-thiazol-2-yl]-benzamide;
4-(Pyridazin-4-ylamino)-N-[4-(4-fluoro-3-trifluoromethyl-phenyl)-thiazol-2-yl]-benzamide;
4-(Pyrazin-2-ylamino)-N-[4-(2-fluoro-3-trifluoromethyl-phenyl)-thiazol-2-yl]-benzamide;
4-(Pyridazin-4-ylamino)-N-[4-(4-fluoro-3-trifluoromethyl-phenyl)-thiazol-2-yl]-benzamide;
4-(1,3,5-Triazin-2-ylamino)-N-[4-(4-fluoro-3-trifluoromethyl-phenyl)-thiazol-2-yl]-benzamide;
N-[4-(2-Fluoro-3-trifluoromethyl-phenyl)-thiazol-2-yl]-4-(pyrimidin-5-ylamino)-benzamide;
N-[4-(2-Fluoro-3-trifluoromethyl-phenyl)-thiazol-2-yl]-4-(pyridazin-3-ylamino)-benzamide;
N-[4-(2-Fluoro-3-trifluoromethyl-phenyl)-thiazol-2-yl]-4-(1,3,4-triazin-3-ylamino)-benzamide;
4-(6-Dimethylamino-pyrimidin-4-ylamino)-N-[4-(2-fluoro-3-trifluoromethyl-phenyl)-thiazol-2-yl]-benzamide;
4-(6-Azetidin-1-yl-pyrimidin-4-ylamino)-N-[4-(2-fluoro-3-trifluoromethyl-phenyl)-thiazol-2-yl]-benzamide;
N-[4-(2-Fluoro-3-trifluoromethyl-phenyl)-thiazol-2-yl]-4-(6-pyrrolidin-1-yl-pyrimidin-4-ylamino)-benzamide;
4-(6-Dimethylamino-pyrimidin-4-ylamino)-N-[4-(2-fluoro-3-trifluoromethyl-phenyl)-thiazol-2-yl]-benzamide;
4-(6-Chloro-pyrimidin-4-ylamino)-N-[4-(2-fluoro-3-trifluoromethyl-phenyl)-thiazol-2-yl]-benzamide;
4-(6-Chloro-pyrimidin-4-ylamino)-N-[4-(4-fluoro-3-trifluoromethyl-phenyl)-thiazol-2-yl]-benzamide;
4-(6-Ethylamino-pyrimidin-4-ylamino)-N-[4-(2-fluoro-3-trifluoromethyl-phenyl)-thiazol-2-yl]-benzamide;
4-(6-Cyclopropylamino-4-ylamino)-N-[4-(2-fluoro-3-trifluoro methyl-phenyl)-thiazol-2-yl]-benzamide;
N-[4-(2-Fluoro-3-trifluoromethyl-phenyl)-thiazol-2-yl]-4-(6-piperidin-1-yl-pyrimidin-4-ylamino)-benzamide;
N-[4-(2-Fluoro-3-trifluoromethyl-phenyl)-thiazol-2-yl]-4-(6-methyl-pyrimidin-4-ylamino)-benzamide;
4-(2,6-Dichloro-pyrimidin-4-ylamino)-N-[4-(2-fluoro-3-trifluoromethyl-phenyl)-thiazol-2-yl]-benzamide;
4-(2-Chloro-6-methyl-pyrimidin-4-ylamino)-N-[4-(2-fluoro-3-trifluoromethyl-phenyl)-thiazol-2-yl]-benzamide;
4-(2,6-Dimethyl-pyrimidin-4-ylamino)-N-[4-(2-fluoro-3-trifluoromethyl-phenyl)-thiazol-2-yl]-benzamide;
4-(2,6-Dimethyl-pyrimidin-4-ylamino)-N-[4-(3,4-difluoro-phenyl)-thiazol-2-yl]-benzamide;
4-(6-Chloropyrimidin-4-ylamino)-N-(3-(4-fluoro-3-(trifluoromethyl)phenyl)-1,2,4-thiadiazol-5-yl)benzamide;
4-(6-(N-(2-Methoxyethyl)-N-methylamino)pyrimidin-4-ylamino)-N-(3-(4-fluoro-3-(trifluoromethyl)phenyl)-1,2,4-thiadiazol-5-yl)benzamide;
4-(6-(N-(2-Methoxyethyl)-N-methylamino)pyrimidin-4-ylamino)-N-(4-(2-fluoro-3-(trifluoromethyl)phenyl)thiazol-2-yl)benzamide;
N-(4-(2,4-Difluoro-3-(trifluoromethyl)phenyl)thiazol-2-yl)-4-(pyrimidin-4-ylamino)benzamide;
N-(5-(2-Fluoro-3-(trifluoromethoxy)phenyl)-1,2,4-thiadiazol-3-yl)-4-(pyrimidin-4-ylamino)benzamide;
N-(3-(2-Fluoro-5-(trifluoromethoxy)phenyl)-1,2,4-thiadiazol-5-yl)-4-(pyrimidin-4-ylamino)benzamide;
4-(6-(Dimethylamino)pyrimidin-4-ylamino)-N-(3-(2-fluoro-5-(trifluoromethoxy)phenyl)-1,2,4-thiadiazol-5-yl)benzamide;
N-(4-(2-Fluoro-3-methoxyphenyl)thiazol-2-yl)-4-(pyrimidin-4-ylamino)benzamide;
4-(6-(N-Methoxy-N-methylamino)pyrimidin-4-ylamino)-N-(3-(4-fluoro-3-(trifluoromethyl)phenyl)-1,2,4-thiadiazol-5-yl)benzamide;
4-(6-(Methylamino)pyrimidin-4-ylamino)-N-(3-(4-fluoro-3-(trifluoromethyl)phenyl)-1,2,4-thiadiazol-5-yl)benzamide;
N-(3-Phenylisothiazol-5-yl)-4-(pyrimidin-4-ylamino)benzamide;
N-(3-(4-Chlorophenyl)isothiazol-5-yl)-4-(pyrimidin-4-ylamino)benzamide;
N-(3-(3,4,5-Trifluorophenyl)-1,2,4-thiadiazol-5-yl)-4-(pyrimidin-4-ylamino)benzamide;
N-(3-(3-Chloro-4-fluorophenyl)-1,2,4-thiadiazol-5-yl)-4-(pyrimidin-4-ylamino)benzamide;
N-(3-(2-Fluoro-5-(trifluoromethyl)phenyl)-1,2,4-thiadiazol-5-yl)-4-(pyrimidin-4-ylamino)benzamide;
N-(3-(2-Fluoro-3-(trifluoromethoxy)phenyl)-1,2,4-thiadiazol-5-yl)-4-(pyrimidin-4-ylamino)benzamide;
4-(6-((S)-2-Hydroxy-2-((S)-2,2-dimethyl-1,3-dioxolan-4-yl)ethylamino)pyrimidin-4-ylamino)-N-(3-(4-fluoro-3-(trifluoromethyl)phenyl)-1,2,4-thiadiazol-5-yl)benzamide;
4-(6-((2S,3S)-2,3,4-Trihydroxybutylamino)pyrimidin-4-ylamino)-N-(3-(4-fluoro-3-(trifluoromethyl)phenyl)-1,2,4-thiadiazol-5-yl)benzamide;
4-(6-(N-(2-(Dimethylamino)ethyl)-N-methylamino)pyrimidin-4-ylamino)-N-(3-(4-fluoro-3-(trifluoromethyl)phenyl)-1,2,4-thiadiazol-5-yl)benzamide;
4-(6-(N-(2-(Hydroxy)ethyl)-N-methylamino)pyrimidin-4-ylamino)-N-(3-(4-fluoro-3-(trifluoromethyl)phenyl)-1,2,4-thiadiazol-5-yl)benzamide;
4-(6-(N-(2,3-(Dihydroxy)propyl)-N-methylamino)pyrimidin-4-ylamino)-N-(3-(4-fluoro-3-(trifluoromethyl)phenyl)-1,2,4-thiadiazol-5-yl)benzamide;
4-(6-(N-(2,3-(Dihydroxy)propyl)-amino)pyrimidin-4-ylamino)-N-(3-(4-fluoro-3-(trifluoromethyl)phenyl)-1,2,4-thiadiazol-5-yl)benzamide;
4-{6-[Bis-(2-hydroxy-ethyl)-amino]-pyrimidin-4-ylamino}-N-[3-(4-fluoro-3-trifluoromethyl-phenyl)-[1,2,4]thiadiazol-5-yl]-benzamide;
4-(6-(1,3-Dihydroxypropan-2-ylamino)pyrimidin-4-ylamino)-N-(3-(4-fluoro-3-(trifluoromethyl)phenyl)-1,2,4-thiadiazol-5-yl)benzamide;
4-(6-((R)-1-Hydroxypropan-2-ylamino)pyrimidin-4-ylamino)-N-(3-(4-fluoro-3-(trifluoromethyl)phenyl)-1,2,4-thiadiazol-5-yl)benzamide;
4-(6-((S)-1-Hydroxypropan-2-ylamino)pyrimidin-4-ylamino)-N-(3-(4-fluoro-3-(trifluoromethyl)phenyl)-1,2,4-thiadiazol-5-yl)benzamide;
4-{6-[Bis-(3-hydroxy-propyl)-amino]-pyrimidin-4-ylamino}-N-[3-(4-fluoro-3-trifluoromethyl-phenyl)-[1,2,4]thiadiazol-5-yl]-benzamide;
4-(6-(3-Hydroxypropylamino)pyrimidin-4-ylamino)-N-(3-(4-fluoro-3-(trifluoromethyl)phenyl)-1,2,4-thiadiazol-5-yl)benzamide;
4-(6-(2-Hydroxyethylamino)pyrimidin-4-ylamino)-N-(3-(4-fluoro-3-(trifluoromethyl)phenyl)-1,2,4-thiadiazol-5-yl)benzamide;
4-(6-(2,3-Dihydroxypropylthio)pyrimidin-4-ylamino)-N-(3-(4-(4-fluoro-3-(trifluoromethyl)phenyl)-1,2,4-thiadiazol-5-yl)benzamide;
4-{6-[N-(2R and 2S)-(2,3-Dihydroxy-propyl)-N-methyl-amino]-pyrimidin-4-ylamino}-N-[3-(4-fluoro-3-trifluoromethyl-phenyl)-[1,2,4]thiadiazol-5-yl]-benzamide;
4-(2-Methylpyrimidin-4-ylamino)-N-(4-(2-fluoro-3-(trifluoromethyl)phenyl)thiazol-2-yl)benzamide;
N-(3-(3,5-Bis(trifluoromethyl)phenyl)-1,2,4-thiadiazol-5-yl)-4-(pyrimidin-4-ylamino)benzamide;
N-(5-(2-Fluoro-3-(trifluoromethyl)phenyl)thiazol-2-yl)-4-(pyrimidin-4-ylamino)benzamide;
N-(5-(4-Fluoro-3-(trifluoromethyl)phenyl)thiazol-2-yl)-4-(pyrimidin-4-ylamino)benzamide;
4-(1,3,4-Thiadiazol-2-ylamino)-N-(4-(2-fluoro-3-(trifluoromethyl)phenyl)thiazol-2-yl)benzamide;
N-[1-(4-Fluoro-3-trifluoromethyl-phenyl)-1H-pyrazol-3-yl]-4-(pyrimidin-4-ylamino)-benzamide;
N-[1-(2-Fluoro-3-trifluoromethyl-phenyl)-1H-pyrazol-3-yl]-4-(pyrimidin-4-ylamino)-benzamide;
4-(6-Cyclopropylmethoxy-pyrimidin-4-ylamino)-N-[3-(4-fluoro-3-trifluoromethyl-phenyl)-[1,2,4]thiadiazol-5-yl]-benzamide;
4-[6-(2-Benzyloxy-ethoxy)-pyrimidin-4-ylamino]-N-[3-(4-fluoro-3-trifluoromethyl-phenyl)-[1,2,4]thiadiazol-5-yl]-benzamide;
N-[3-(4-Fluoro-3-trifluoromethyl-phenyl)-[1,2,4]thiadiazol-5-yl]-4-(6-methoxy-pyrimidin-4-ylamino)-benzamide;
N-[3-(4-Fluoro-3-trifluoromethyl-phenyl)-[1,2,4]thiadiazol-5-yl]-4-(6-isopropoxy-pyrimidin-4-ylamino)-benzamide;
4-(6-Ethoxy-pyrimidin-4-ylamino)-N-[3-(4-fluoro-3-trifluoromethyl-phenyl)-[1,2,4]thiadiazol-5-yl]-benzamide;
N-[3-(4-Fluoro-3-trifluoromethyl-phenyl)-[1,2,4]thiadiazol-5-yl]-4-[6-(2-methoxy-ethoxy)-pyrimidin-4-ylamino]-benzamide;
N-[3-(4-Fluoro-3-trifluoromethyl-phenyl)-[1,2,4]thiadiazol-5-yl]-4-[6-(2-methoxy-1-methyl-ethoxy)-pyrimidin-4-ylamino]-benzamide;
4-(6-Cyclobutylmethoxy-pyrimidin-4-ylamino)-N-[3-(4-fluoro-3-trifluoromethyl-phenyl)-[1,2,4]thiadiazol-5-yl]-benzamide;
N-[3-(4-Fluoro-3-trifluoromethyl-phenyl)-[1,2,4]thiadiazol-5-yl]-4-[2-(2-methoxy-1-methyl-ethoxy)-pyridin-4-ylamino]-benzamide;
4-[6-(Azetidin-3-yloxy)-pyrimidin-4-ylamino]-N-[3-(4-fluoro-3-trifluoromethyl-phenyl)-[1,2,4]thiadiazol-5-yl]-benzamide;
4-(6-Cyclohexylmethoxy-pyrimidin-4-ylamino)-N-[3-(4-fluoro-3-trifluoromethyl-phenyl)-[1,2,4]thiadiazol-5-yl]-benzamide;
N-[3-(4-Fluoro-3-trifluoromethyl-phenyl)-[1,2,4]thiadiazol-5-yl]-4-(2-methoxy-pyridin-4-ylamino)-benzamide;
N-[3-(4-Fluoro-3-trifluoromethyl-phenyl)-[1,2,4]thiadiazol-5-yl]-4-[6-(2-hydroxy-ethoxy)-pyrimidin-4-ylamino]-benzamide;
N-[3-(4-Fluoro-3-trifluoromethyl-phenyl)-[1,2,4]thiadiazol-5-yl]-4-(1H-[1,2,3]triazolo[4,5-d]pyrimidin-7-ylamino)-benzamide;
N-[3-(4-Fluoro-3-trifluoromethyl-phenyl)-[1,2,4]thiadiazol-5-yl]-4-(9H-purin-6-ylamino)-benzamide;
N-[3-(4-Fluoro-3-trifluoromethyl-phenyl)-[1,2,4]thiadiazol-5-yl]-4-(1H-[1,2,3]triazolo[4,5-d]pyrimidin-7-ylamino)-benzamide;
4-(2-Chloro-pyridin-4-ylamino)-N-[3-(4-fluoro-3-trifluoromethyl-phenyl)-[1,2,4]thiadiazol-5-yl]-benzamide;
4-(6-Chloro-pyrimidin-4-ylamino)-N-[4-(4-fluoro-3-trifluoromethyl-phenyl)-5-methyl-thiazol-2-yl]-benzamide;

4 N-[3-(3-Bromo-4-fluoro-phenyl)-[1,2,4]thiadiazol-5-yl]-4-(6-chloro-pyrimidin-4-ylamino)-benzamide;
4-(6-Chloro-pyrimidin-4-ylamino)-N-[5-methyl-4-(3-trifluoromethyl-phenyl)-thiazol-2-yl]-benzamide;
4-(6-Chloro-pyrimidin-4-ylamino)-N-[4-(2-fluoro-3-trifluoromethyl-phenyl)-5-methyl-thiazol-2-yl]-benzamide;
4-(6-Chloro-pyrimidin-4-ylamino)-N-[4-(2-fluoro-5-trifluoromethyl-phenyl)-5-methyl-thiazol-2-yl]-benzamide;
N-[3-(3-Butyl-4-fluoro-phenyl)-[1,2,4]thiadiazol-5-yl]-4-(6-chloro-pyrimidin-4-ylamino)-benzamide;
4-(6-Isopropoxy-pyrimidin-4-ylamino)-N-[3-(4-isopropoxy-3-trifluoromethyl-phenyl)-[1,2,4]thiadiazol-5-yl]-benzamide;
4-(6-Ethoxy-pyrimidin-4-ylamino)-N-[3-(4-ethoxy-3-trifluoromethyl-phenyl)-[1,2,4]thiadiazol-5-yl]-benzamide;
4-(6-Methoxy-pyrimidin-4-ylamino)-N-[3-(4-methoxy-3-trifluoromethyl-phenyl)-[1,2,4]thiadiazol-5-yl]-benzamide;
N-[3-(4-Fluoro-3-trifluoromethyl-phenyl)-[1,2,4]thiadiazol-5-yl]-4-(6-pyrrolidin-1-yl-pyrimidin-4-ylamino)-benzamide;
N-[3-(4-Fluoro-3-trifluoromethyl-phenyl)-[1,2,4]thiadiazol-5-yl]-4-(6-methoxy-pyridin-3-ylamino)-benzamide;
N-[3-(4-Fluoro-3-trifluoromethyl-phenyl)-[1,2,4]thiadiazol-5-yl]-4-(pyridin-4-ylamino)-benzamide;
4-(2-Ethoxy-pyridin-4-ylamino)-N-[3-(4-fluoro-3-trifluoromethyl-phenyl)-[1,2,4]thiadiazol-5-yl]-benzamide;
4-(2-Cyclopropylmethoxy-pyridin-4-ylamino)-N-[3-(4-fluoro-3-trifluoromethyl-phenyl)-[1,2,4]thiadiazol-5-yl]-benzamide;
N-[3-(4-Fluoro-3-trifluoromethyl-phenyl)-[1,2,4]thiadiazol-5-yl]-4-(5-trifluoromethyl-pyridin-2-ylamino)-benzamide;
N-[4-(2-Fluoro-3-trifluoromethyl-phenyl)-5-methyl-thiazol-2-yl]-4-{6-[3-(4-methyl-piperazin-1-yl)-propylamino]-pyrimidin-4-ylamino}-benzamide;
N-[4-(2-Fluoro-3-trifluoromethyl-phenyl)-5-methyl-thiazol-2-yl]-4-{6-[3-(2-oxo-pyrrolidin-1-yl)-propylamino]-pyrimidin-4-ylamino}-benzamide;
4-{6-[(2,3-Dihydroxy-propyl)-methyl-amino]-pyrimidin-4-ylamino}-N-[4-(2-fluoro-3-trifluoromethyl-phenyl)-5-methyl-thiazol-2-yl]-benzamide;
4-(6-Dimethylamino-pyrimidin-4-ylamino)-N-[4-(2-fluoro-3-trifluoromethyl-phenyl)-5-methyl-thiazol-2-yl]-benzamide;
N-[4-(2-Fluoro-3-trifluoromethyl-phenyl)-5-methyl-thiazol-2-yl]-4-(6-methylamino-pyrimidin-4-ylamino)-benzamide;
4-(6-Dimethylamino-pyrimidin-4-ylamino)-N-[5-methyl-4-(3-trifluoromethyl-phenyl)-thiazol-2-yl]-benzamide;
4-[6-(2,3-Dihydroxy-propylamino)-pyrimidin-4-ylamino]-N-[4-(2-fluoro-3-trifluoromethyl-phenyl)-5-methyl-thiazol-2-yl]-benzamide;
4-[6-(4-Ethyl-piperazin-1-yl)-pyrimidin-4-ylamino]-N-[4-(2-fluoro-3-trifluoromethyl-phenyl)-5-methyl-thiazol-2-yl]-benzamide;
4-(6-Methylamino-pyrimidin-4-ylamino)-N-[5-methyl-4-(3-trifluoromethyl-phenyl)-thiazol-2-yl]-benzamide;
4-[6-(2,3-Dihydroxy-propylamino)-pyrimidin-4-ylamino]-N-[5-methyl-4-(3-trifluoromethyl-phenyl)-thiazol-2-yl]-benzamide;
4-[6-(2,3-Dihydroxy-propylamino)-pyrimidin-4-ylamino]-N-[5-methyl-4-(3-trifluoromethyl-phenyl)-thiazol-2-yl]-benzamide;
4-(6-Dimethylamino-pyrimidin-4-ylamino)-N-[5-methyl-4-(3-trifluoromethyl-phenyl)-thiazol-2-yl]-benzamide;
4-(6-Dimethylamino-pyrimidin-4-ylamino)-N-[4-(2-fluoro-5-trifluoromethyl-phenyl)-5-methyl-thiazol-2-yl]-benzamide;
4-{6-[(2,3-Dihydroxy-propyl)-methyl-amino]-pyrimidin-4-ylamino}-N-[4-(2-fluoro-5-trifluoromethyl-phenyl)-5-methyl-thiazol-2-yl]-benzamide;
4-[6-(2,3-Dihydroxy-propylamino)-pyrimidin-4-ylamino]-N-[4-(4-fluoro-3-trifluoromethyl-phenyl)-5-methyl-thiazol-2-yl]-benzamide;
N-[4-(4-Fluoro-3-trifluoromethyl-phenyl)-5-methyl-thiazol-2-yl]-4-(6-methylamino-pyrimidin-4-ylamino)-benzamide;
N-[4-(4-Fluoro-3-trifluoromethyl-phenyl)-5-methyl-thiazol-2-yl]-4-(6-methylamino-pyrimidin-4-ylamino)-benzamide;
N-[4-(4-Fluoro-3-trifluoromethyl-phenyl)-5-methyl-thiazol-2-yl]-4-(6-methylamino-pyrimidin-4-ylamino)-benzamide;
N-[4-(4-Fluoro-3-trifluoromethyl-phenyl)-5-methyl-thiazol-2-yl]-4-{6-[3-(4-methyl-piperazin-1-yl)-propylamino]-pyrimidin-4-ylamino}-benzamide;
4-(6-Dimethylamino-pyrimidin-4-ylamino)-N-[4-(4-fluoro-3-trifluoromethyl-phenyl)-5-methyl-thiazol-2-yl]-benzamide;
4-{6-[(2,3-Dihydroxy-propyl)-methyl-amino]-pyrimidin-4-ylamino}-N-[4-(4-fluoro-3-trifluoromethyl-phenyl)-5-methyl-thiazol-2-yl]-benzamide;
N-[3-(3-Butyl-4-fluoro-phenyl)-[1,2,4]thiadiazol-5-yl]-4-(6-dimethylamino-pyrimidin-4-ylamino)-benzamide;
4-{6-[Bis-(3-hydroxy-propyl)-amino]-pyrimidin-4-ylamino}-N-[3-(3-butyl-4-fluoro-phenyl)-[1,2,4]thiadiazol-5-yl]-benzamide;
2-Butyl-4-[4-(2-{4-[3-(1,3-dimethyl-pentyl)-benzyl]-phenyl}-allyl)-cyclopenta-1,4-dienyl]-1-methyl-benzene;
N-[3-(3-Butyl-4-fluoro-phenyl)-[1,2,4]thiadiazol-5-yl]-4-(6-methylamino-pyrimidin-4-ylamino)-benzamide;
N-[3-(3-Butyl-4-fluoro-phenyl)-[1,2,4]thiadiazol-5-yl]-4-{6-[3-(2-oxo-pyrrolidin-1-yl)-propylamino]-pyrimidin-4-ylamino}-benzamide;
N-[5-Methyl-4-(2-fluoro-3-trifluoromethyl-phenyl)-thiazol-2-yl]-4-(pyrimidin-4-ylamino)-benzamide;

4 N-[4-(4-Fluoro-3-trifluoromethyl-phenyl)-5-methyl-thiazol-2-yl]-4-(pyrimidin-4-ylamino)-benzamide;
N-[5-Methyl-4-(3-trifluoromethyl-phenyl)-thiazol-2-yl]-4-(pyrimidin-4-ylamino)-benzamide;
N-[3-(3-Bromo-4-fluoro-phenyl)-[1,2,4]thiadiazol-5-yl]-4-(pyrimidin-4-ylamino)-benzamide;
N-[4-(3-ethyl-2-fluoro-phenyl)-thiazol-2-yl]-4-(pyrimidin-4-ylamino)-benzamide;
N-[4-(3-Butyl-2-fluoro-phenyl)-thiazol-2-yl]-4-(pyrimidin-4-ylamino)-benzamide;
N-[4-(2-Fluoro-3-isobutyl-phenyl)-thiazol-2-yl]-4-(pyrimidin-4-ylamino)-benzamide;
N-[3-(3-Butyl-4-fluoro-phenyl)-[1,2,4]thiadiazol-5-yl]-4-(pyrimidin-4-ylamino)-benzamide;
N-[4-(2-Fluoro-5-trifluoromethyl-phenyl)-5-methyl-thiazol-2-yl]-4-(pyrimidin-4-ylamino)-benzamide;
4-(6-(3-Hydroxyazetidin-1-yl)pyrimidin-4-ylamino)-N-(4-(2-fluoro-3-(trifluoromethoxy)phenyl)thiazol-2-yl)benzamide;
4-(6-(3-Hydroxyazetidin-1-yl)pyrimidin-4-ylamino)-N-(3-(4-fluoro-3-(trifluoromethyl)phenyl)-1,2,4-thiadiazol-5-yl)benzamide;

(R)N-[4-(2-fluoro-3-trifluoromethoxy-phenyl)-thiazol-2-yl]-4-{6-[(2,3-dihydroxy-propyl)-methyl-amino]-pyrimidin-4-ylamino}-benzamide;

(S)N-[4-(2-fluoro-3-trifluoromethoxy-phenyl)-thiazol-2-yl]-4-{6-[(2,3-dihydroxy-propyl)-methyl-amino]-pyrimidin-4-ylamino}-benzamide; and
N-[4-(2-fluoro-3-trifluoromethyl-phenyl)-thiazol-2-yl]-4-{6-[(2-hydroxy-ethyl)-methyl-amino]-pyrimidin-4-ylamino}-benzamide.

With regard to the terms R³(C₁-C₆)alkyl, R⁷(C₁-C₆)alkoxy, R¹⁵(C₁-C₆)alkyl and similar terms used throughout this disclosure such as, e.g., R³(C₁-C₆)alkoxy, R¹¹(C₁-C₆)alkoxycarbonyl, R¹⁴(C₂-C₉)heteroaryl, R¹⁴(C₃-C₁₀)cycloalkyl, R¹⁵(C₁-C₆)alkoxy, etc., those of skill in the art will appreciate that when R³, R⁷, or R¹⁵, etc. (or more generally referred to here as “R group(s)”) is hydrogen, the moiety to which the R group is attached is effectively unsubstituted by a group other than hydrogen. In that regard, when such terms are substituted by a certain number of R groups and the R groups are hydrogen, other hydrogen atoms that may already be present on the moiety to which the R groups are attached continue to be present. For example, in the term R³(C₁-C₆)alkyl, where R³ is three groups selected from hydrogen and (C₁-C₆)alkyl is a n-butyl radical, the resulting group is n-butyl having the chemical formula C₄H₉. In another example, in the term R¹⁴(C₆-C₁₀)aryl, wherein R¹⁴ is two groups selected from hydrogen and (C₆-C₁₀)aryl is a phenyl radical, the resulting group is phenyl radical having the chemical formula C₆H₅. Of course, other variations will be readily apparent to those of skill in the art given the benefit of the present disclosure.

The present invention further relates to a pharmaceutical composition for (a) treating or preventing a disorder or condition selected from decreased megakaryopoiesis and platelet numbers, decreased hematopoietic stem cells, decreased erythopoiesis and myelopoiesis; aiding bone marrow repopulation after bone marrow or cord blood transplant; expanding megakaryocyte and stem cell numbers in vitro prior to transplant; increasing platelet numbers in normal individuals prior to surgery, cytoreductive chemotherapy, or radiation treatment; increasing platelet numbers in normal individuals prior to platelet pheresis to harvest platelets for later transfusion; increasing platelet numbers in thrombocytopenic patients or (b) treating or preventing a disorder or condition that can be treated or prevented by agonizing the TPO receptor in a mammal, including a human, comprising an amount of a compound of the present invention or a pharmaceutically acceptable salt thereof, effective in such disorders or conditions and a pharmaceutically acceptable carrier.

The present invention further relates to a method for agonizing the TPO receptor in a mammal, including a human, comprising administering to said mammal an effective amount of a compound of the present invention or a pharmaceutically acceptable salt thereof.

The present invention further relates to a method for treating or preventing a disorder or condition selected from decreased megakaryopoiesis and platelet numbers, decreased hematopoietic stem cells, decreased erythopoiesis and myelopoiesis; aiding bone marrow repopulation after bone marrow or cord blood transplant; expanding megakaryocyte and stem cell numbers in vitro prior to transplant; increasing platelet numbers in normal individuals prior to surgery, cytoreductive chemotherapy, or radiation treatment; increasing platelet numbers in normal individuals prior to platelet pheresis to harvest platelets for later transfusion; and increasing platelet numbers in thrombocytopenic patients, in a mammal, including a human, comprising administering to said mammal an amount of a compound of the present invention or a pharmaceutically acceptable salt thereof, effective in treating such a disorder or condition.

The present invention further relates to co-administering a therapeutically effective amount of an agent selected from the group consisting of: a colony stimulating factor, cytokine, chemokine, interleukin or cytokine receptor agonist or antagonists, soluble receptors, receptor agonists or antagonist antibodies, or small molecules or peptides that act by the same mechanisms as one or more of said agents. In certain embodiments, the agent is selected from the group consisting of: G-CSF, GM-CSF, TPO, M-CSF, EPO, Gro-beta, IL-11, SCF, FLT3 ligand, LIF, 1 L-3, IL-6, IL-1, Progenipoietin, NESP, SD-01, IL-8, or IL-S or a biologically active derivative of any of said agents.

The present invention further relates to a method for enhancing platelet production obtained from a donor comprising administering to such donor a therapeutically effective amount of a compound of the present invention or a pharmaceutically acceptable salt thereof prior to platelet pheresis, blood donation or platelet donation.

The present invention further relates to a method for enhancing the number of peripheral blood stem cells obtained from a donor comprising administering to such donor a therapeutically effective amount of a compound of the present invention or a pharmaceutically acceptable salt thereof prior to leukapheresis. In certain embodiments, the method further comprises co-administering a therapeutically effective amount of a hematopoietic-cell mobilizing agent selected from the group consisting of: a colony stimulating factor, cytokine, chemokine, interleukin or cytokine receptor agonist, adhesion molecule antagonists or antibodies. In certain embodiments, the mobilizing agent is selected from the group consisting of: G-CSF, GM-CSF, TPO, EPO, Gro-beta, 1L-8, cytoxan, VLA-4 inhibitors, SCF, FLT3 ligand or a biologically active derivative of G-CSF, GM-CSF, TPO, EPO, Gro-beta or 1 L-8. In certain embodiments, the agent causes terminal differentiation in certain types of hematopoietic malignancies.

With regard to certain terms used herein to describe the presently disclosed methods, compositions, biological effects, etc., such as “decreased”, “increasing”, “normal”, as used in the phrases “decreased hematopoietic stem cells”, “increasing platelet numbers”, and “normal individuals”, respectively, it should be understood that such terms are used in a relative qualitative sense based on a quantitative departure from the norm. In that regard, the “norm” is indicative of a “normal individual” recognized by those of skill in the art and may vary amongst individuals depending on, e.g., the demographic group of which the individual is a member, size, weight, gender, etc.

### DEFINITIONS

As used herein, the term “pharmaceutically acceptable salt” means either a pharmaceutically acceptable acid addition salt or a pharmaceutically acceptable base addition salt of a currently disclosed compound that may be administered without any resultant substantial undesirable biological effect(s) or any resultant deleterious interaction(s) with any other component of a pharmaceutical composition in which it may be contained.

As used herein, the term “(C₁-C₆)alkyl” means a saturated linear or branched free radical consisting essentially of 1 to 6 carbon atoms and a corresponding number of hydrogen atoms. Exemplary (C₁-C₆)alkyl groups include methyl, ethyl, n-propyl, isopropyl, n-butyl, isobutyl, etc. Of course, other (C₁-C₆)alkyl groups will be readily apparent to those of skill in the art given the benefit of the present disclosure.

As used herein, the term “(C₃-C₁₀)cycloalkyl” means a nonaromatic saturated free radical forming at least one ring consisting essentially of 3 to 10 carbon atoms and a corresponding number of hydrogen atoms. As such, (C₃-C₁₀)cycloalkyl groups can be monocyclic or multicyclic. Individual rings of such multicyclic cycloalkyl groups can have different connectivities, e.g., fused, bridged, spiro, etc. in addition to covalent bond substitution. Exemplary (C₃-C₁₀)cycloalkyl groups include cyclopropyl, cyclobutyl, cyclopentyl, cyclohexyl, norbornanyl, bicyclo[3.2.1]octanyl, octahydro-pentalenyl, spiro[4.5]decanyl, cyclopropyl substituted with cyclobutyl, cyclobutyl substituted with cyclopentyl, cyclohexyl substituted with cyclopropyl, etc. Of course, other (C₃-C₁₀)cycloalkyl groups will be readily apparent to those of skill in the art given the benefit of the present disclosure.

As used herein, the term “(C₂-C₉)heterocycloalkyl” means a nonaromatic free radical having 3 to 10 atoms (i.e., ring atoms) that form at least one ring, wherein 2 to 9 of the ring atoms are carbon and the remaining ring atom(s) (i.e., hetero ring atom(s)) is selected from the group consisting of nitrogen, sulfur, and oxygen. As such, (C₂-C₉)heterocycloalkyl groups can be monocyclic or multicyclic. Individual rings of such multicyclic heterocycloalkyl groups can have different connectivities, e.g., fused, bridged, spiro, etc. in addition to covalent bond substitution. Exemplary (C₂-C₉)heterocycloalkyl groups include pyrrolidinyl, tetrahydrofuranyl, dihydrofuranyl, tetrahydropyranyl, pyranyl, thiopyranyl, aziridinyl, azetidinyl, oxiranyl, methylenedioxyl, chromenyl, barbituryl, isoxazolidinyl, 1,3-oxazolidin-3-yl, isothiazolidinyl, 1,3-thiazolidin-3-yl, 1,2-pyrazolidin-2-yl, 1,3-pyrazolidin-1-yl, piperidinyl, thiomorpholinyl, 1,2-tetrahydrothiazin-2-yl, 1,3-tetrahydrothiazin-3-yl, tetrahydrothiadiazinyl, morpholinyl, 1,2-tetrahydrodiazin-2-yl, 1,3-tetrahydrodiazin-1-yl, tetrahydroazepinyl, piperazinyl, piperizin-2-onyl, piperizin-3-onyl, chromanyl, 2-pyrrolinyl, 3-pyrrolinyl, imidazolidinyl, 2-imidazolidinyl, 1,4-dioxanyl, 8-azabicyclo[3.2.1]octanyl, 3-azabicyclo[3.2.1]octanyl, 3,8-diazabicyclo[3.2.1]octanyl, 2,5-diazabicyclo[2.2.1]heptanyl, 2,5-diazabicyclo[2.2.2]octanyl, octahydro-2H-pyrido[1,2-a]pyrazinyl, 3-azabicyclo[4.1.0]heptanyl, 3-azabicyclo[3.1.0]hexanyl-2-azaspiro[4.4]nonanyl, 7-oxa-1-aza-spiro[4.4]nonanyl, 7-azabicyclo[2.2.2]heptanyl, octahydro-1H-indolyl, etc. In general, the (C₂-C₉)heterocycloalkyl group typically is attached to the main structure via a carbon atom or a nitrogen atom. Of course, other (C₂-C₉)heterocycloalkyl groups will be readily apparent to those of skill in the art given the benefit of the present disclosure.

As used herein, the term “(C₂-C₉)heteroaryl” means an aromatic free radical having 5 to 10 atoms (i.e., ring atoms) that form at least one ring, wherein 2 to 9 of the ring atoms are carbon and the remaining ring atom(s) (i.e., hetero ring atom(s)) is selected from the group consisting of nitrogen, sulfur, and oxygen. As such, (C₂-C₉)heteroaryl groups can be monocyclic or multicyclic. Individual rings of such multicyclic heteroaryl groups can have different connectivities, e.g., fused, etc. in addition to covalent bond substitution. Exemplary (C₂-C₉)heteroaryl groups include furyl, thienyl, thiazolyl, pyrazolyl, isothiazolyl, oxazolyl, isoxazolyl, pyrrolyl, triazolyl, tetrazolyl, imidazolyl, 1,3,5-oxadiazolyl, 1,2,4-oxadiazolyl, 1,2,3-oxadiazolyl, 1,3,5-thiadiazolyl, 1,2,3-thiadiazolyl, 1,2,4-thiadiazolyl, pyridyl, pyrimidyl, pyrazinyl, pyridazinyl, 1,2,4-triazinyl, 1,2,3-triazinyl, 1,3,5-triazinyl, pyrazolo[3,4-b]pyridinyl, cinnolinyl, pteridinyl, purinyl, 6,7-dihydro-5H-[1]pyrindinyl, benzo[b]thiophenyl, 5,6,7,8-tetrahydro-quinolin-3-yl, benzoxazolyl, benzothiazolyl, benzisothiazolyl, benzisoxazolyl, benzimidazolyl, thianaphthenyl, isothianaphthenyl, benzofuranyl, isobenzofuranyl, isoindolyl, indolyl, indolizinyl, indazolyl, isoquinolyl, quinolyl, phthalazinyl, quinoxalinyl, quinazolinyl and benzoxazinyl, etc. In general, the (C₂-C₉)heteroaryl group typically is attached to the main structure via a carbon atom, however, those of skill in the art will realize when certain other atoms, e.g., hetero ring atoms, can be attached to the main structure. Of course, other (C₂-C₉)heteroaryl groups will be readily apparent to those of skill in the art given the benefit of the present disclosure.

As used herein, the term “(C₆-C₁₀)aryl” means phenyl or naphthyl.

As used herein, the term “halo” means fluorine, chlorine, bromine, or iodine.

As used herein, the term “amino” means a free radical having a nitrogen atom and 1 to 2 hydrogen atoms. As such, the term amino generally refers to primary and secondary amines. In that regard, as used herein and in the appended claims, a tertiary amine is represented by the general formula RR′N—, wherein R and R′ are carbon radicals that may or may not be identical. Nevertheless, the term “amino” generally may be used herein to describe a primary, secondary, or tertiary amine, and those of skill in the art will readily be able to ascertain the identification of which in view of the context in which this term is used in the present disclosure.

### Abbreviations

### ACN refers to acetonitrile.

### DMF refers to N,N-dimethylformamide.

### DMSO refers to dimethylsulfoxide.

### EtOAc refers to ethyl acetate.

### EtOH refers to ethanol.

### Hunig's Base refers to diisopropylethyl amine (“DIPEA”).

### MeOH refers to methanol.

### NaOH refers to sodium hydroxide.

### THF refers to tetrahydrofuran.

### TFA refers to trifluoroacetic acid.

Additional features and advantages of compounds disclosed herein will be apparent from the following detailed description of certain embodiments.

### DETAILED DESCRIPTION OF THE INVENTION

Although specific embodiments of the present disclosure will now be described with reference to the preparations and schemes, it should be understood that such embodiments are by way of example only and merely illustrative of but a small number of the many possible specific embodiments which can represent applications of the principles of the present disclosure. Various changes and modifications will be obvious to those of skill in the art given the benefit of the present disclosure and are deemed to be within the spirit and scope of the present disclosure as further defined in the appended claims.

Unless defined otherwise, all technical and scientific terms used herein have the same meaning as commonly understood by one having ordinary skill in the art to which this disclosure belongs. Although other compounds or methods can be used in practice or testing, certain preferred methods are now described in the context of the following preparations and schemes.

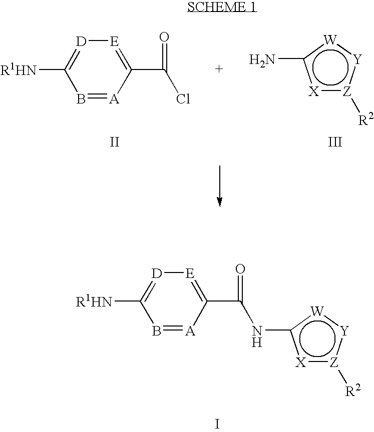

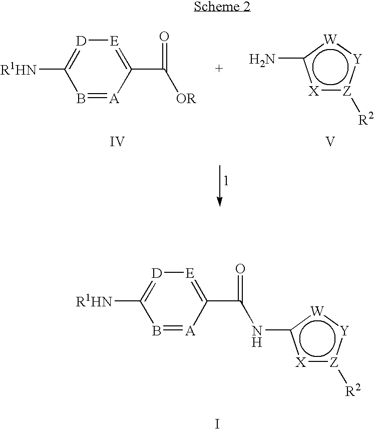

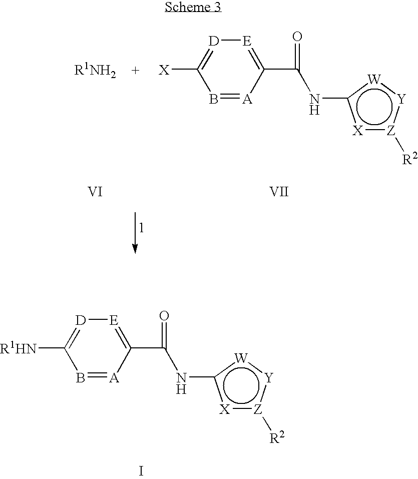

In reaction 1 of Scheme 1, the compound of formula II is reacted with the amine compound of formula III in an aprotic solvent, such as pyridine to give the compound of formula I. The reaction is stirred at a temperature between about 70° C. to about 90° C., preferably about 80° C., for a time period between about 15 hours to about 20 hours, preferably about 18 hours.

In reaction 1 of Scheme 2, the amine compound of formula IV, wherein R is (C₁-C₆)alkyl or benzyl, preferably methyl, is reacted with the amine compound of formula V in the presence of trimethylaluminum or diisopropylaluminum hydride, and an aprotic solvent, such as toluene, methylene chloride or dichloroethane, preferably methylene chloride, to form the compound of formula I. The reaction is stirred at a temperature between about room temperature to about 150° C., preferably about 100° C. to about 120° C., for a time period from about 1 hour to about 20 hours, preferably from about 1 hour to about 2 hours in a sealed vessel at microwave or from about 10 hours to about 20 hours at reflux.

In reaction 1 of Scheme 3, the amine compound of formula VI is reacted with the compound of formula VII, wherein X is bromo, iodo or triflylate, in the presence of (a) a palladium catalyst, such as palladium acetate or palladium dibenzylidene acetone (Pd(dba)₃), (b) a ligand capable of complexing with palladium, such as a phosphene or an imidazolidinium salt, preferably Xantphos®, (c) a base, such as cesium carbonate, sodium tert-butoxide or potassium phosphate, preferably cesium carbonate, and (d) an aprotic solvent, such as dioxane or tetrahydrofuran, preferably dioxane, to form the compound of formula I. The reaction is stirred at a temperature between about room temperature to reflux, preferably at reflux, for a time period from about 1 hour to about 48 hours, preferably about 20 hours.

All pharmaceutically acceptable salts, prodrugs, tautomers, hydrates and solvates of a compound of the invention are also encompassed by the invention.

A compound of the invention which is basic in nature is capable of forming a wide variety of different salts with various inorganic and organic acids. Although such salts must be pharmaceutically acceptable for administration to animals and humans, it is often desirable in practice to initially isolate a compound of the invention from the reaction mixture as a pharmaceutically unacceptable salt and then simply convert the latter back to the free base compound by treatment with an alkaline reagent, and subsequently convert the free base to a pharmaceutically acceptable acid addition salt. The acid addition salts of the base compounds of this invention are readily prepared by treating the base compound with a substantially equivalent amount of the chosen mineral or organic acid in an aqueous solvent medium or in a suitable organic solvent such as, for example, methanol or ethanol. Upon careful evaporation of the solvent, the desired solid salt is obtained.

The acids which can be used to prepare the pharmaceutically acceptable acid addition salts of the base compounds of this invention are those which form non-toxic acid addition salts, i.e., salts containing pharmacologically acceptable anions, such as chloride, bromide, iodide, nitrate, sulfate or bisulfate, phosphate or acid phosphate, acetate, lactate, citrate or acid citrate, tartrate or bitartrate, succinate, maleate, fumarate, gluconate, saccharate, benzoate, methanesulfonate and pamoate [i.e., 1,1′-methylene-bis-(2-hydroxy-3-naphthoate)] salts.

A compound of the invention which is also acidic in nature, e.g., contains a COOH or tetrazole moiety, is capable of forming base salts with various pharmacologically acceptable cations. Although such salts must be pharmaceutically acceptable for administration to animals and humans, it is often desirable in practice to initially isolate a compound of the invention from the reaction mixture as a pharmaceutically unacceptable salt and then simply convert the latter back to the free acid compound by treatment with an acidic reagent, and subsequently convert the free acid to a pharmaceutically acceptable base addition salt. Examples of such pharmaceutically acceptable base addition salts include the alkali metal or alkaline-earth metal salts and particularly, the sodium and potassium salts. These salts can be prepared by conventional techniques. The chemical bases which can be used as reagents to prepare the pharmaceutically acceptable base addition salts of this invention are those which form non-toxic base salts with the herein described acidic compounds of the invention. These non-toxic base salts include salts derived from such pharmacologically acceptable cations as sodium, potassium, calcium and magnesium, etc. These salts can easily be prepared by treating the corresponding acidic compounds with an aqueous solution containing the desired pharmacologically acceptable cations, and then evaporating the resulting solution to dryness, preferably under reduced pressure. Alternatively, they may also be prepared by mixing lower alkanolic solutions of the acidic compounds and the desired alkali metal alkoxide together, and then evaporating the resulting solution to dryness in the same manner as before. In either case, stoichiometric quantities of reagents are preferably employed in order to ensure completeness of reaction and maximum product yields.

Isotopically-labeled compounds are also encompassed by the present invention. As used herein, an “isotopically-labeled compound” refers to a compound of the invention including pharmaceutical salts, prodrugs thereof, each as described herein, in which one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

By isotopically-labeling a compound of the present invention, the compounds may be useful in drug and/or substrate tissue distribution assays. Tritiated (³H) and carbon-14 (¹⁴C) labeled compounds are particularly preferred for their ease of preparation and detectability. Further, substitution with heavier isotopes such as deuterium (²H) can afford certain therapeutic advantages resulting from greater metabolic stability, for example increased in vivo half-life or reduced dosage requirements and, hence, may be preferred in some circumstances. Isotopically labeled compounds of the invention, including pharmaceutical salts, prodrugs thereof, can be prepared by any means known in the art.

Stereoisomers (e.g., cis and trans isomers) and all optical isomers of a compound of the invention (e.g., R and S enantiomers), as well as racemic, diastereomeric and other mixtures of such isomers are contemplated by the present invention.

The compounds, salts, prodrugs, hydrates, and solvates of the present invention can exist in several tautomeric forms, including the enol and imine form, and the keto and enamine form and geometric isomers and mixtures thereof. All such tautomeric forms are included within the scope of the present invention. Tautomers exist as mixtures of a tautomeric set in solution. In solid form, usually one tautomer predominates. Even though one tautomer may be described, the present invention includes all tautomers of the present compounds.

The present invention also includes atropisomers of the present invention. Atropisomers refer to compounds of the invention that can be separated into rotationally restricted isomers.

The present invention also provides a pharmaceutical composition comprising at least one compound of the invention and at least one pharmaceutically acceptable carrier. A pharmaceutical composition of the invention may be prepared by conventional means known in the art including, for example, mixing at least one compound of the invention with a pharmaceutically acceptable carrier. The pharmaceutically acceptable carrier may be any such carrier known in the art including those described in, for example, Remington's Pharmaceutical Sciences, Mack Publishing Co., (A. R. Gennaro edit. 1985).

A pharmaceutical composition of the invention may be used in the prevention or treatment in an animal or human. Thus, a compound of the invention may be formulated as a pharmaceutical composition for oral, buccal, intranasal, parenteral (e.g., intravenous, intramuscular or subcutaneous), topical, or rectal administration or in a form suitable for administration by inhalation or insufflation.

For oral administration, the pharmaceutical composition may take the form of, for example, a tablet or capsule prepared by conventional means with a pharmaceutically acceptable excipient such as a binding agent (e.g., pregelatinized maize starch, polyvinylpyrrolidone or hydroxypropyl methylcellulose); filler (e.g., lactose, microcrystalline cellulose or calcium phosphate); lubricant (e.g., magnesium stearate, talc or silica); disintegrant (e.g., potato starch or sodium starch glycolate); or wetting agent (e.g., sodium lauryl sulphate). The tablets may be coated by methods well known in the art. Liquid preparations for oral administration may take the form of a, for example, solution, syrup or suspension, or they may be presented as a dry product for constitution with water or other suitable vehicle before use. Such liquid preparations may be prepared by conventional means with a pharmaceutically acceptable additive such as a suspending agent (e.g., sorbitol syrup, methyl cellulose or hydrogenated edible fats); emulsifying agent (e.g., lecithin or acacia); non-aqueous vehicle (e.g., almond oil, oily esters or ethyl alcohol); and preservative (e.g., methyl or propyl p-hydroxybenzoates or sorbic acid).

For buccal administration, the composition may take the form of tablets or lozenges formulated in conventional manner.

A compound of the present invention may also be formulated for sustained delivery according to methods well known to those of ordinary skill in the art. Examples of such formulations can be found in U.S. Pat. Nos. 3,538,214; 4,060,598; 4,173,626; 3,119,742; and 3,492,397, which are herein incorporated by reference in their entirety.

A compound of the invention may be formulated for parenteral administration by injection, including using conventional catheterization techniques or infusion. Formulations for injection may be presented in unit dosage form, e.g., in ampules or in multi-dose containers, with an added preservative. The compositions may take such forms as suspensions, solutions or emulsions in oily or aqueous vehicles, and may contain a formulating agent such as a suspending, stabilizing and/or dispersing agent. Alternatively, the active ingredient may be in powder form for reconstitution with a suitable vehicle, e.g., sterile pyrogen-free water, before use.

A compound of the invention may also be formulated in rectal compositions such as suppositories or retention enemas, e.g., containing conventional suppository bases such as cocoa butter or other glycerides.

For intranasal administration or administration by inhalation, a compound of the invention may be conveniently delivered in the form of a solution or suspension from a pump spray container that is squeezed or pumped by the patient or as an aerosol spray presentation from a pressurized container or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. The pressurized container or nebulizer may contain a solution or suspension of the compound of the invention. Capsules and cartridges (made, for example, from gelatin) for use in an inhaler or insufflator may be formulated containing a powder mix of a compound of the invention and a suitable powder base such as lactose or starch.

A proposed dose of a compound of the invention for oral, parenteral or buccal administration to the average adult human for the treatment of a TPO-related disease state is about 0.1 mg to about 2000 mg, preferably, about 0.1 mg to about 200 mg of the active ingredient per unit dose which could be administered, for example, 1 to 4 times per day.

Aerosol formulations for treatment of the conditions referred to above in the average adult human are preferably arranged so that each metered dose or “puff” of aerosol contains about 20 μg to about 10,000 μg, preferably, about 20 μg to about 1000 μg of a compound of the invention. The overall daily dose with an aerosol will be within the range from about 100 μg to about 100 mg, preferably, about 100 μg to about 10 mg. Administration may be several times daily, for example 2, 3, 4 or 8 times, giving for example, 1, 2 or 3 doses each time.

Aerosol combination formulations for treatment of the conditions referred to above in the average adult human are preferably arranged so that each metered dose or “puff” of aerosol contains from about 0.01 mg to about 1000 mg, preferably, about 0.01 mg to about 100 mg of a compound of this invention, more preferably from about 1 mg to about 10 mg of such compound. Administration may be several times daily, for example 2, 3, 4 or 8 times, giving for example, 1, 2 or 3 doses each time. Aerosol formulations for treatment of the conditions referred to above in the average adult human are preferably arranged so that each metered dose or “puff” of aerosol contains from about 0.01 mg to about 20,000 mg, preferably, about 0.01 mg to about 2000 mg of a compound of the invention, more preferably from about 1 mg to about 200 mg. Administration may be several times daily, for example 2, 3, 4 or 8 times, giving for example, 1, 2 or 3 doses each time.

For topical administration, a compound of the invention may be formulated as an ointment or cream.

This invention also encompasses pharmaceutical compositions containing and methods of treatment or prevention comprising administering prodrugs of at least one compound of the invention. As used herein, the term “prodrug” means a pharmacological derivative of a parent drug molecule that requires biotransformation, either spontaneous or enzymatic, within the organism to release the active drug. Prodrugs are variations or derivatives of the compounds of this invention which have groups cleavable under metabolic conditions. Prodrugs become the compounds of the invention which are pharmaceutically active in vivo, when they undergo solvolysis under physiological conditions or undergo enzymatic degradation. Prodrug compounds of this invention may be called single, double, triple etc., depending on the number of biotransformation steps required to release the active drug within the organism, and indicating the number of functionalities present in a precursor-type form. Prodrug forms often offer advantages of solubility, tissue compatibility, or delayed release in the mammalian organism (see, Bundgard, Design of Prodrugs, pp. 7-9, 21-24, Elsevier, Amsterdam 1985 and Silverman, The Organic Chemistry of Drug Design and Drug Action, pp. 352-401, Academic Press, San Diego, Calif., 1992). Prodrugs commonly known in the art include acid derivatives well known to practitioners of the art, such as, for example, esters prepared by reaction of the parent acids with a suitable alcohol, or amides prepared by reaction of the parent acid compound with an amine, or basic groups reacted to form an acylated base derivative. Moreover, the prodrug derivatives of this invention may be combined with other features herein taught to enhance bioavailability. For example, a compound of the invention having free amino, amido, hydroxy or carboxylic groups can be converted into prodrugs. Prodrugs include compounds wherein an amino acid residue, or a polypeptide chain of two or more (e.g., two, three or four) amino acid residues which are covalently joined through peptide bonds to free amino, hydroxy or carboxylic acid groups of compounds of the invention. The amino acid residues include the 20 naturally occurring amino acids commonly designated by three letter symbols and also include, 4-hydroxyproline, hydroxylysine, demosine, isodemosine, 3-methylhistidine, norvalin, beta-alanine, gamma-aminobutyric acid, citrulline homocysteine, homoserine, ornithine and methionine sulfone. Prodrugs also include compounds wherein carbonates, carbamates, amides and alkyl esters which are covalently bonded to the above substituents of a compound of the invention through the carbonyl carbon prodrug sidechain.

The following Examples illustrate the preparation of the compounds of the present invention. Melting points are uncorrected. NMR data are reported in parts per million (d) and are referenced to the deuterium lock signal from the sample solvent (deuterochloroform unless otherwise specified). Mass Spectral data were obtained using a Micromass ZMD APCI Mass Spectrometer equipped with a Gilson gradient high performance liquid chromatograph. The following solvents and gradients were used for the analysis. Solvent A; 98% water/2% acetonitrile/0.01% formic acid and solvent B; acetonitrile containing 0.005% formic acid. Typically, a gradient was run over a period of about 4 minutes starting at 95% solvent A and ending with 100% solvent B. The mass spectrum of the major eluting component was then obtained in positive or negative ion mode scanning a molecular weight range from 165 AMU to 1100 AMU. Specific rotations were measured at room temperature using the sodium D line (589 nm). Commercial reagents were utilized without further purification. THF refers to tetrahydrofuran. DMF refers to N,N-dimethylformamide. Chromatography refers to column chromatography performed using 32-63 mm silica gel and executed under nitrogen pressure (flash chromatography) conditions. Room or ambient temperature refers to 20-25° C. All non-aqueous reactions were run under a nitrogen atmosphere for convenience and to maximize yields. Concentration at reduced pressure means that a rotary evaporator was used.

One of ordinary skill in the art will appreciate that in some cases protecting groups may be required during preparation. After the target molecule is made, the protecting group can be removed by methods well known to those of ordinary skill in the art, such as described in Greene and Wuts, Protective Groups in Organic Synthesis (2^{nd} Ed, John Wiley & Sons 1991).

Analytical high performance liquid chromatography on reverse phase with mass spectrometry detection (LSMS) was done using Polaris 2×20 mm C18 column. Gradient elution was applied with increase of concentration of acetonitrile in 0.01% aqueous formic acid from 5% to 100% during 3.75 min period. Mass spectrometer Micromass ZMD was used for molecular ion identification.

### Reporter Assay

A murine hematopoeitic IL3 dependent cell line BaF3 transfected with the human TPO receptor (TPOr) and the STAT1/3 responsive β-lactamase reporter was used to assess the agonist activity of the presently disclosed compounds against the TPO receptor in the present assay. In particular, the present assay measures the induction of the β-lactamase enzymatic activity in response to TPOr stimulation. CCF4/AM, a membrane-permeant substrate ester derived from CCF4 and a fluorescent substrate for β-lactamases, was added to the cells to monitor the observed activity because it is known that as CCF4/AM is accumulated intracellularly in mammalian cells, CCF4/AM is converted to CCF4 by endogenous cytoplasmic esterases. The substrate fluoresces green (530 nm), and the product of its β-lactamase catalyzed hydrolysis fluoresces blue (460 nm).

The transfected BaF3 IL-3 dependent cell line was maintained in RPMI (Gibco, #12376-018), 10% heat inactivated fetal bovine serum (Hyclone SH30070.03), 250 ug/ml Zeocyn (Invitrogen, #204281), 0.5 mg/ml Geneticin (Gibco, #10131-035), 10 ng/ml hTpo (R&D Systems, 288-TP-025), and 1% Penicillin-Streptomycin. The cells were split 1:5 three times per week. Approximately 12 hours before initiating the assay, the cells were washed three times for about 10 minutes at about 500×g and the media was replaced with phenol red free RPMI (Gibco, #11835-030) with 10% FBS without hTPO for about 18 hours.

Drug dilutions were prepared in RPMI and 0.1% BSA (“assay media”) and were subsequently delivered in triplicate 20 μL of compound into a 384-well Costar clear bottom, black plate (VWR, #29444-080) using a BioMek (Beckman-Coulter). Columns 1-18 were reserved for drug dilutions. Columns 19-22 were used as control columns. In particular, column 19 contained cells and 300 ng/mL Peprotech hTPO; column 20 contained cells and 100 ng/mL mlL3; column 21 contained cells and assay media; and column 22 contained only assay media. The cells were washed three times for about 10 minutes (each wash) at 500×g in a solution of phenol red free RPMI and assay media. After the final wash, the cells were resuspended in about 10 mL of assay media and counted using Trypan Blue. 20 uL of cells were added to columns 1-20 of the 384-well plate using a Multi-drop (ThermoLabSystems) for a final cell concentration of 10,000 cells per well. The plate was spun at about 300×g for about 1 second. Incubation occurred for about 5 hours at about 37° C. under 5% CO₂.

A loading solution was prepared from three solutions (Solution A=1 mM CCF4/AM in DMSO; Solution B and Solution C were provided by Aurora Biomed, Inc.) in the following proportions: for each mL of loading solution, 6 μL Solution A was added to 60 μL Solution B and vortexed. 1 mL of Solution C was subsequently mixed with the foregoing solution. 10 μL of the loading solution was added to each well of the 384-well plate via the Multi-drop. The plate was agitated for about two seconds using a horizontal plate shaker. Incubation proceeded in the dark at room temperature for about 1 hour. Activity was detected on a LJL Analyst (Molecular Devices) equipped with the 405-20 excitation filter, 2 emission filters (blue channel 460-40 and green channel 530-10) and a 425 dichroic. The Stimulation Index was as follows: [(460/530 ratio drug samples/460/530 No Stimulation Ratio)]-1. The reported EC₅₀ values were calculated by plotting SI ratio drug against SI ratio hTPO control.

All of the exemplified compounds had an EC₅₀ value of less than 50 μM in the Reporter Assay.

### EXPERIMENTAL

### Preparation A

### 4-(Pyrimidin-4-ylamino)-benzoyl Chloride Hydrochloride

### Step 1: 4-(6-Chloropyrimidin-4-ylamino)-benzoic acid

4-(6-Dichloropyrimidine (15.0 g, 100.6 mmol) and 4-aminobenzoic acid (17.0 g, 106.8 mmol) were heated at reflux in a mixture of conc. HCl (2 mL), water (65 mL) and acetone (45 mL) for 2.5 h. After standing for a few hours at room temperature, the solid was collected, washed with acetone and dried yielding 22.7 g (90%) of the title compound as a white solid.

### Step 2: 4-(Pyrimidin-4-ylamino)-benzoic acid

4-(6-Chloropyrimidin-4-ylamino)-benzoic acid (22.5 g, 78.6 mmol) was hydrogenated over 10% Pd on carbon for 3 h at 45 psi at room temperature in methanol (750 mL). The reaction mixture was then filtered and the filtrate concentrated. The residue was triturated with CH₂Cl₂/hexane. The solid was collected and dried yielding 12.2 g (62%) of the title compound as a yellow solid.

### Step 3: 4-(Pyrimidin-4-ylamino)-benzoyl chloride hydrochloride

4-(Pyrimidin-4-ylamino)-benzoic acid (4.30 g, 20 mmol) and thionyl chloride (8 mL) were heated at reflux in dioxane (36 mL) for 3 h. The reaction mixture was then concentrated and the solid residue triturated with ether. Collection and drying yielded 4.78 g (88%) of the title compound as a pale yellow solid. LC-MS m/z (M+H)⁺ 230, (M−H)⁻ 228.

### Preparation B

### 4-(6-Azetidin-1-yl-pyrimidin-4-ylamino)-benzoic Acid Methyl Ester

### Step 1: 4-Azetidin-1-yl-6-chloro-pyrimidine

4-(6-Dichloropyrimidine (745 mg, 5.0 mmol), azetidine hydrochloride (561 mg, 6.0 mmol), and diisopropylethylamine (1.55 g, 12.0 mmol) were heated at 60° C. for 18 h in isopropanol (50 mL). The cooled mixture was diluted with CH₂Cl₂ and washed with saturated ammonium chloride solution, dried over MgSO₄, filtered and concentrated to a solid. This was triturated with ether, collected and dried yielding 706 mg (83%) of the title compound as a white solid.

### Step 2: 4-(6-Azetidin-1-yl-pyrimidin-4-ylamino)-benzoic acid

4-Azetidin-1-yl-6-chloro-pyrimidine (700 mg, 4.1 mmol) and 4-aminobenzoic acid (1.13 g, 8.25 mmol) were heated at 70° C. in a mixture of conc. HCl (83 μL), water (0.75 mL), and butanone (3 mL) for 4 days. The cooled mixture was concentrated yielding the crude title compound as a damp, brown solid.

### Step 3: 4-(6-Azetidin-1-yl-pyrimidin-4-ylamino)-benzoic acid methyl ester

4-(6-Azetidin-1-yl-pyrimidin-4-ylamino)-benzoic acid (≦4.1 mmol) was suspended in a mixture of toluene (5 mL) and methanol (5 mL) and treated with excess TMSCHN₂. After 30 min. the reaction was concentrated and the residue chromatographed yielding 322 mg (28%) of the title compound as a white solid. LC-MS m/z (M+H)⁺ 285, (M−H)⁻ 283.

### Preparation C

### 4-(6-Dimethylamino-pyrimidin-4-ylamino)-benzoic Acid Methyl Ester

### Step 1: 4-(6-Chloro-pyrimidin-4-ylamino)-benzoic acid

4-(6-Dichloropyrimidine (7.45 g, 50 mmol), 4-aminobenzoic acid (7.21 g, 53 mmol) were heated at 90° C. in a mixture of conc. HCl (1 mL), water (32 mL), and acetone (22 mL) for 4 h. The cooled mixture was filtered yielding 9.74 g (78%) of the title compound as a white solid.

### Step 2: 4-(6-Dimethylamino-pyrimidin-4-ylamino)-benzoic acid

4-(6-Chloro-pyrimidin-4-ylamino)-benzoic acid (474 mg, 1.0 mmol) and dimethylamine (1.0 mL of 2.0 M solution in methanol, 2.0 mmol), and triethylamine (303 mg, 3.0 mmol) were heated at 100° C. for 18 h in dioxane (15 mL) in a sealed reaction tube. The mixture was concentrated yielding the crude title compound as a white solid.

### Step 3: 4-(6-Dimethylamino-pyrimidin-4-ylamino)-benzoic acid methyl ester

4-(6-Dimethylamino-pyrimidin-4-ylamino)-benzoic acid (≦1.0 mmol) was suspended in a mixture of toluene (10 mL) and methanol (10 mL) and treated with excess TMSCHN₂. After 60 min. the reaction was concentrated and the residue chromatographed yielding 334 mg (65%) of the title compound as an off-white solid. LC-MS m/z (M+H)⁺ 273, (M+HCO2H—H)⁻ 317.

### Preparation D

### (3-Dimethylcarbamoyimethylsulfanyl-[1,2,4]thiadiazol-5-yl)-(4-methoxy-benzyl)-carbamic Acid tert-butyl Ester

### Step 1: 2-Carbamimidoylsulfanyl-N,N-dimethyl-acetamide

2-Chloro-N,N-dimethyl-acetamide (16.27 g, 134 mmol) and thiourea (10.19 g, 134 mmol) were combined in acetone and stirred at room temperature for 16 h. The resulting solid was collected and dried yielding 24.20 g (91%) of the title compound as a white powder.

### Step 2: 2-(5-Amino-[1,2,4]thiadiazol-3-ylsulfanyl)-N N-dimethyl-acetamide

Sodium thiocyanate (11.91 g, 147 mmol) was dissolved in methanol (180 mL). To this was added 2-carbamimidoylsulfanyl-N,N-dimethyl-acetamide (24.20 g, 122 mmol) followed by the simultaneous addition of solutions of bromine (19.5 g, 122 mmol) in methanol (60 mL) and sodium methoxide (from 5.86 g, 244 mmol of sodium) in methanol (120 mL) with vigorous stirring at −15° C. After complete addition the mixture was stirred 1 h and then was poured into a mixture of water (1 L) and saturate ammonium chloride solution (0.5 L). After 30 min, the solid was collected washed with water and dried yielding 6.39 g (24%) of the title compound as a light yellow powder.

### Step 3: (3-Dimethylcarbamoylmethylsulfanyl-[1,2,4]thiadiazol-5-yl)-carbamic acid tert-butyl ester

2-(5-Amino-[1,2,4]thiadiazol-3-ylsulfanyl)-N,N-dimethyl-acetamide (1.09 g, 5.0 mmol), di-t-butyldicarbonate (1.31 g, 6.0 mmol) and 4-dimethylaminopyridine (60 mg, 0.5 mmol) were combined in THF (25 mL) and stirred at room temperature for 72 h and at 60° C. for 8 h. The mixture was concentrated and the residue taken up in ethyl acetate and washed with 1N HCl and saturated sodium bicarbonate solution. After drying over MgSO₄, filtration and concentration a yellow solid was obtained which was chromatographed yielding 946 mg (59%) of the title compound as a light yellow powder.

### Step 4: (3-Dimethylcarbamoylmethylsulfanyl-[1,2,4]thiadiazol-5-yl)-(4-methoxy-benzyl)-carbamic acid tert-butyl Ester

(3-Dimethylcarbamoylmethylsulfanyl-[1,2,4]thiadiazol-5-yl)-carbamic acid tert-butyl ester (930 mg, 2.92 mmol), 4-methoxybenzyl chloride (915 mg, 5.84 mmol) and DBU (667 mg, 4.38 mmol) were heated together at 80° C. in dioxane for 4 h. The reaction mixture was diluted with ethyl acetate, washed with 1N HCl and dried over MgSO₄. Filtration and concentration gave a thick gum which was chromatographed yielding 860 mg (67%) of the title compound as a thick oil that crystallized on standing. LC-MS m/z (M+H)⁺ 439, (M-C₄H, CO₂)⁻ 337.

### Preparation E

### 4-(Pyrimidin-4-ylamino)-benzoic Acid Methyl Ester

4-(Pyrimidin-4-ylamino)-benzoyl chloride hydrochloride (5.40 g, 20 mmol/from Preparation A) was stirred in methanol (40 mL) for 18 h at room temperature. The solvent was removed and the solid triturated with ether. The solid was collected and then partitioned between CH₂Cl₂ and saturated sodium bicarbonate solution. The organic layer was separated, dried over MgSO₄, filtered and concentrated yielding 2.88 g (63%) of the title compound as a pale yellow solid. LC-MS m/z (M+H)⁺ 230, (M−H)⁻ 228.

### Preparation F

### (3-Methylsulfanyl-[1,2,4]thiadiazol-5-yl)-(4-methoxy-benzyl)-carbamic Acid tert-butyl Ester

### Step 1: (3-Methylsulfanyl-[1,2,4-]thiadiazol-5-yl)-carbamic acid tert-butyl ester

3-Methylsulfanyl-[1,2,4]thiadiazol-5-ylamine (1.47 g, 10.0 mmol) and di-t-butyldicarbonate (2.62 g, 12.0 mmol) in THF (75 mL) was treated with sodium hexamethyldisilazide (24 mL of 1.0 M solution in THF, 24 mmol) over a few minutes with ice cooling. After 4 h the reaction was diluted with ethyl acetate and washed with saturated ammonium chloride solution and dried over MgSO₄. Filtration and concentration gave a damp solid which was triturated with hexanelether yielding 684 mg (27%) of the title compound as a white solid. Concentration and chromatography of the filtrate gave 760 mg (31%) of the title compound as a white solid.

### Step 2: (3-Methylsulfanyl-[1,2,4]thiadiazol-5-yl)-(4-methoxy-benzyl)-carbamic acid tert-butyl ester

(3-Methylsulfanyl-[1,2,4]thiadiazol-5-yl)-carbamic acid tert-butyl ester (1.75 g, 7.06 mmol), 4-methoxybenzyl chloride (2.09 g, 14 mmol) and DBU (1.61 g, 10.6 mmol) were heated together at 80° C. in dioxane for 3 h. The reaction mixture was diluted with ethyl acetate and washed with 1N HCl and dried over MgSO₄. Filtration and concentration gave a thick gum which was chromatographed yielding 2.08 g (80%) of the title compound as a white solid. LC-MS m/z (M+H)⁺ 368.

### Preparation G

### N,N-Dimethyl-N′-(5-methylsulfanyl-[1,2,4]thiadiazol-3-yl)-formamidine

5-Methylsulfanyl-[1,2,4]thiadiazol-3-ylamine (2.94 g, 20 mmol) and dimethylformamide dimethyl acetal (3.57 g, 30 mmol) were heated at 80° C. for 4 h in dioxane. The cooled reaction was concentrated and the residue triturated in ether/hexane. The solid was collected and dried yielding 3.88 g (96%) of the title compound as an off-white solid. APCI-MS m/z (M+H)⁺ 203, APCI-MS m/z (M-CH₃—H)⁻ 203.

### Preparation H

### Trifluoro-methanesulfonic Acid 2-[tert-butoxycarbonyl-(4-methoxy-benzyl)-amino]-thiazol-4-yl Ester

### Step 1: (4-Oxo-4,5-dihydro-thiazol-2-yl)-carbamic acid tert-butyl ester

Pseudothiohydantoin (5.82 g, 50 mmol) and di-t-butyl dicarbonate (21.82 g, 100 mmol) were combined in dry THF (100 mL) and stirred at 60° C. for 48 h. The cooled mixture was treated with decolorizing carbon and filtered through diatomaceous earth rinsing with THF. The filtrate was concentrated to a damp solid which was triturated with hexane. The resulting solid was collected and rinsed with hexane yielding 9.35 g (86%) of the title compound as a light tan solid.

### Step 2: Trifluoro-methanesulfonic acid 2-tert-butoxycarbonylamino-thiazol-4-yl ester

(4-Oxo-4,5-dihydro-thiazol-2-yl)-carbamic acid tert-butyl ester (3.84 g, 17.7 mmol) and 2,6-lutidine (5.70 g, 53.2 mmol) were combined in dry CH₂Cl₂ and cooled in an ice bath. To this was added by syringe, neat trifluoromethanesulfonic anhydride (10.0 g, 35.4 mmol) over a few minutes. After 1 h the reaction was quenched with saturated NH₄Cl solution (100 mL) and the organic layer separated. The organic layer was washed with saturated sodium bicarbonate solution (50 mL). Silica gel (˜8 g) was added to the organic layer and the mixture concentrated to dryness. The resulting solid was charged onto a column and chromatographed yielding 5.18 g of the title compound as an off-white solid.

### Step 3: Trifluoro-methanesulfonic Acid 2-[tert-butoxycarbonyl-(4-methoxy-benzyl)-amino]-thiazol-4-yl ester

Trifluoro-methanesulfonic acid 2-tert-butoxycarbonylamino-thiazol-4-yl ester (5.17 g, 14.8 mmol), 4-methoxybenzyl chloride (4.64 g, 29.6 mmol) and DBU (3.38 g, 22.2 mmol) were combined in dry dioxane (75 mL) and heated to 80° C. After 3 h the reaction was cooled to room temperature, diluted with ether and washed with 1N HCl and dried with MgSO₄. The extract was filtered and concentrated to a yellow oil which was chromatographed yielding a thick oil that crystallized on standing. After trituration with hexane the solid was collected yielding 5.41 g (78%) of the title compound as a white crystalline solid. LC-MS m/z (M+H)⁺ 469.

### Preparation I

### 3-(4-Fluoro-3-trifluoromethyl-phenyl)-[1,2,4]thiadiazol-5-ylammonium Chloride

### Step 1: 4-Fluoro-3-trifluoromethyl-benzonitrile

A solution of 4-Fluoro-3-trifluoromethyl-benzonitrile (10 g, 52.9 mmol) sodium methoxide (10.6 mL of a 0.5 M solution in methanol, 5.3 mmol; 0.1 eq) in methanol (40 mL) was allowed to stir 12-36 h at room temperature. Acetic acid (0.32 g, 5.3 mmol) was added followed by NH₄Cl (2.8 g, 52.9 mmol). The reaction was stirred at 50° C. for 24 h. The reaction was cooled, the unreacted ammonium chloride removed by filtration and the resultant white solid was used without purification 9 g (82%).

### Step 2: 3-(4-Fluoro-3-trifluoromethyl-phenyl)-[1,2,4]thiadiazol-5-ylamine

A portion of the above white solid (6 g, 24.8 mmol) was dissolved in methanol (50 mL) and was cooled to −5° C. To this was added bromine (3.0 g, 24.8 mmol) over a period of 5 min taking care to keep the reaction below −5° C. Potassiumthiocyanate (2.4 g 24.8 mmol) was added over 1 min while keeping the reaction below 5° C. Both of these additions are somewhat exothermic. To this mixture was added a freshly prepared solution of sodium methoxide in methanol (prepared from sodium (1.14 g, 49.6 mmol) and methanol (30 mL)) resulting in the formation of a white precipitate. The reaction was allowed to warm to room temperature and stirred for 3 h. The reaction was concentrated to ⅓ of the volume and poured into water (150 mL) with the formation of a different white precipitate. This was allowed to stir for 1 h and the precipitate was collected by filtration to provide 3.5 g (53%) of the title compound as a white solid.

### Step 3: 3-(4-Fluoro-3-trifluoromethyl-phenyl)-[1,2,4]thiadiazol-5-yl-ammonium chloride

HCl gas was bubbled into a solution of 3-(4-Fluoro-3-trifluoromethyl-phenyl)-[1,2,4]thiadiazol-5-yl-amine in ether. A white solid formed and the solvent was removed from the slurry by evaporation to yield the title compound as a white solid that was used without further purification.

### Preparation J

### 2-Ethoxy-pyridin-4-ylamine

A solution of sodium (0.28 g, 12.2 mmol) in ethanol (3 mL) was added to 2-chloro-pyridin-4-ylamine (0.2 g, 1.56 mmol) in a sealed tube and the reaction was heated to 140° C. for 9 h. The cooled mixture was adjusted to pH 8-9 with 2N HCl. The mixture was extracted with 80:20 chloroform: 2-propanol, concentrated and chromatographed to yield 0.14 g (64%) of the title compound as a white solid.

### Preparation K

### N-[3-(4-Fluoro-3-trifluoromethyl-phenyl)-[1,2,4]thiadiazol-5-yl]-4-iodo-benzamide

4-iodo-benzoyl chloride (0.76 mg, 2.8 mmol) and 3-(4-Fluoro-3-trifluoromethyl-phenyl)-[1,2,4]thiadiazol-5-ylamine (0.5 g, 1.9 mmol) were added to a flame dried conical flask. The reaction vessel was twice evacuated and then flushed with nitrogen. Pyridine (2.0 mL) was added and the reaction placed in an oil bath at 105° C. for 1 h. Upon cooling the reaction mixture was transferred to a round bottom flask and adsorbed onto silica gel. Chromatography yielded 0.8 g (86%) of the title compound as an off-white solid.

### Preparation L

### 4-(2-Fluoro-3-trifluoromethyl-phenyl)-5-methyl-thiazol-2-ylammonium Chloride

### Step 1: 2-Chloro-1-(2-fluoro-3-trifluoromethyl-phenyl)-propan-1-one

A solution of 1-(2-Fluoro-3-trifluoromethyl-phenyl)-propan-1-one (3.5 g, 15.9 mmol) in sulfuryl chloride (2.3 ml, 28.6 mmol) was stirred at 65° C. for 4 h. While the reaction was not complete by GC-MS, the crude mixture was concentrated and used as is.

### Step 2: 4-(2-Fluoro-3-trifluoromethyl-phenyl)-5-methyl-thiazol-2-ylamine

The above mixture was combined with thiourea (1.45 g, 19.1 mmol) in acetone (60 mL). The reaction was stirred at 40° C. for three days (alternatively, the reaction is complete after 4 h at 60° C.). The reaction was concentrated, diluted with saturated aqueous sodium bicarbonate and extracted with dichloromethane, dried over MgSO₄, concentrated adsorbed onto silica gel and chromatographed to yield 3.1 g (71%) of the titled compound.

### Step 3: 4-(2-Fluoro-3-trifluoromethyl-Phenyl)-5-methyl-thiazol-2-ylammonium chloride

4-(2-Fluoro-3-trifluoromethyl-phenyl)-5-methyl-thiazol-2-ylamine was converted to the corresponding hydrochloride salt in quantitative yield by dissolving in ether and bubbling HCl through the mixture. The resultant white solid was collected upon filtration or concentration.

### Preparation M

### 4-(6-Chloro-pyrimidin-4-ylamino)-N-[4-(2-fluoro-3-trifluoromethyl-phenyl)-5-methyl-thiazol-2-yl]-benzamide

Trimethyl aluminum (0.66 mL of a 2.0 M solution in toluene) was slowly added to a solution of 4-(2-Fluoro-3-trifluoromethyl-phenyl)-5-methyl-thiazol-2-ylammonium chloride (0.98 g, 3.1 mmol) (see Preparation L) in CH₂Cl₂ (12 mL) at 0° C. The reaction was allowed to warm to room temperature over 1.5 h. The reaction was split into 3 microwave vials and to each vial was added 4-(6-Chloro-pyrimidin-4-ylamino)-benzoic acid methyl ester (225 mg, 0.85 mmol) (see Preparation L). Each vial was heated at 120° C. for 50 min in the microwave. The vials were combined, diluted with water and brine and extracted with EtOAc with 5% methanol. The organic extracts were concentrated, adsorbed onto silica gel and chromatographed to provide 0.70 g (44%) of the title compound.

### Preparation N

### 4-(Pyrimidin-4-ylamino)-benzoic Acid Methyl Ester

4-(6-Chloro-pyrimidin-4-ylamino)-benzoic acid methyl ester (1.0 g, 3.8 mmol) was combined with 10% palladium on carbon (0.18 g) in methanol (20 mL) in a parr flask and shaken for 3 h under hydrogen (45 psi). The solvent was removed and the mixture was chromatographed to provide the title compound.

### Preparation O

### 4-(3-Ethyl-2-fluoro-phenyl)-thiazol-2-ylamine

### Step 1: 3-Bromo-2-fluoro-N-methoxy-N-methyl-benzamide

3-Bromo-2-fluoro-benzoic acid (0.22 g, 1.0 mmol), O,N-Dimethyl-hydroxylammonium chloride (0.11 g, 1.1 mmol), carbon tetrabromide (0.32 g, 1.1 mmol), triphenylphosphine (0.29 g, 1.1 mmol) and pyridine (87 mg, 1.1 mmol) were all combined in CH₂Cl₂ (10 mL) and stirred at room temperature for 4 h. The mixture was concentrated and chromatographed to provide 0.19 mg (73%) of the title compound as colorless oil.

### Step 2: 1-(3-Bromo-2-fluoro-phenyl)-ethanone

Methylmagnesium bromide (0.8 mL of a 1.4 M solution in THF/toluene), was added to a solution of 3-Bromo-2-fluoro-N-methoxy-N-methyl-benzamide (190 g, 0.73 mmol) in THF (1 mL) at 0° C. and the reaction was stirred at 0° C. for 2 h. The reaction was quenched with dilute aqueous HCl and extracted with EtOAc. The organics were dried over MgSO₄, concentrated and chromatographed to provide 100 mg (64% yield) of the title compound as colorless oil.

### Step 3: 1-(3-Ethyl-2-fluoro-phenyl)-ethanone

1-(3-Bromo-2-fluoro-phenyl)-ethanone (0.52 g, 2.4 mmol), Ethyl boronic acid (0.23 g, 3.1 mmol), potassium phosphate (1.5 g, 7.2 mmol), tricyclohexlyphosphine (70 mg, 0.24 mmol), palladium acetate (28 mg, 0.12 mmol) were combined in toluene (10 mL) and water (0.5 mL) and then heated to 100° C. for 3 h. The reaction cooled, water added and the mixture was extracted with EtOAc. The combined organics were dried over MgSO₄, concentrated and chromatographed to provide 210 mg (52% yield) of the titled compound as colorless oil.

### Example 1

### N-[2-(4-Fluoro-3-trifluoromethyl-phenyl)-oxazol-4-yl]-4-(pyrimidin-4-ylamino)-benzamide

### Step A: 2-(4-Fluoro-3-trifluoromethyl-phenyl)-oxazole-4-carboxylic acid methyl ester

Utilizing the procedure of Phillips et al. (Organic Letters 2000, 2, 1165) 2-(4-fluoro-3-trifluoromethyl-benzoylamino)-3-hydroxy-propionic acid methyl ester (618 mg, 2.0 mmol) was converted into 475 mg (82%) of the title compound as a white solid.

### Step B: 2-(4-Fluoro-3-trifluoromethyl-Phenyl)-oxazole-4-carboxylic acid

Utilizing the procedure of Shafer at al. (Heterocycles 2000, 53, 1167) 2-(4-fluoro-3-trifluoromethyl-phenyl)-oxazole-4-carboxylic acid methyl ester (470 mg, 1.63 mmol) was converted into 448 mg (100%) of the title compound as an off-white solid.

### Step C: [2-(4-Fluoro-3-trifluoromethyl-phenyl)-oxazol-4-yl]-carbamic acid tert-butyl ester

Utilizing the procedure of Neville et al. (DT 2459380) 2-(4-fluoro-3-trifluoromethyl-phenyl)-oxazole-4-carboxylic acid (448 mg, 1.63 mmol) was converted into 298 mg (48%) of the title compound as a white solid.

### Step D: [2-(4-Fluoro-3-trifluoromethyl-phenyl)-oxazol-4-yl]-(4-iodo-benzoyl)-carbamic acid tert-butyl ester

Utilizing the procedure of Neville et al. (DT 2459380) [2-(4-fluoro-3-trifluoromethyl-phenyl)-oxazol-4-yl]-carbamic acid tert-butyl ester (295 mg, 0.85 mmol) was converted into 484 mg (99%) of the title compound as a yellow foam.

### Step E: N-[2-(4-Fluoro-3-trifluoromethyl-phenyl)-oxazol-4-yl]-4-iodo-benzamide

Utilizing the procedure of Stafford et al. (Tetrahedron Letters 1993, 34, 7873) [2-(4-fluoro-3-trifluoromethyl-phenyl)-oxazol-4-yl]-(4-iodo-benzoyl)-carbamic acid tert-butyl ester (484 mg, 0.85 mmole) was converted into 254 mg (63%) of the title compound as a yellow solid.

### Step F: N-[2-(4-Fluoro-3-trifluoromethyl-Phenyl)-oxazol-4-yl]-4-(pyrimidin-4-ylamino)-benzamide

N-[2-(4-Fluoro-3-trifluoromethyl-phenyl)-oxazol-4-yl]-4-iodo-benzamide (250 mg, 0.525 mmol), pyrimidin-4-ylamine (75 mg, 0.79 mmol), cesium carbonate (257 mg, 0.79 mmol), Pd₂(dba)₃ (24 mg, 0.026 mmol), and Xantphos® (33 mg, 0.057 mmol) were combined in a dry flask which was then purged with nitrogen. Dry, nitrogen purged dioxane (10 mL) was then added and the mixture heated to 100° C. for 24 h. The cooled mixture was filtered through diatomaceous earth rinsing with THF. Silica gel (˜6 g) was added to the filtrate which was then concentrated to dryness. The residue was charged onto a column and chromatographed (CH₂Cl₂/CH₃OH). The fractions containing product were combined and concentrated to a yellow solid which was triturated with ether. Filtration gave 86 mg (37%) of the title compound as a yellow solid. LC-MS m/z (M+H)⁺ 444, (M−H)⁻ 442.

### Example 2

### N-[2-(2-Fluoro-3-trifluoromethyl-phenyl)-oxazol-4-yl]-4-(Pyrimidin-4-ylamino)-benzamide

Utilizing the same sequence of reactions as described in Example 1 and starting with 2-(2-fluoro-3-trifluoromethyl-benzoylamino)-3-hydroxy-propionic acid methyl ester (in Step A), the title compound was prepared as a tan solid. LC-MS m/z (M+H)⁺ 444, (M−H)⁻ 442.

### Example 3

### N-[2-(2,4-Difluoro-phenyl)-thiazol-4-yl]-4-(Pyrimidin-4-ylamino)-benzamide

### Step A: 2,4-Difluoro-thiobenzamide

2,4-Difluorobenzamide (1.57 g, 10 mmol) and Lawesson's Reagent (2.02 g, 10 mmol) were stirred together at room temperature for 18 h. The reaction mixture was concentrated and chromatographed yielding the title compound 1.74 g (100%) as a solid.

### Step B: 2-(2,4-Difluoro-phenyl)-thiazole-4-carboxylic acid ethyl ester

2,4-Difluoro-thiobenzamide (1.04 g, 6.0 mmol) and ethyl bromopyruvate (1.17 g, 6.0 mmol) were refluxed in ethanol for 3 h. After cooling, the solvent was removed yielding 1.71 g (100%) of the title compound as a crystalline solid.

### Step C: 2-(2,4-Difluoro-phenyl)-thiazole-4-carboxylic acid

2-(2,4-Difluoro-phenyl)-thiazole-4-carboxylic acid ethyl ester (1.62 g, 6.0 mmol) and lithium hydroxide hydrate (0.50 g, 12 mmol) were heated to 80° C. in a mixture of water (6 mL) and THF (7 mL) for 2 h. The cooled mixture was concentrated and the residue dissolved in ethyl acetate. The organic solution was washed with 1M citric acid solution and then dried over MgSO₄. Filtration and concentration gave 1.83 g of the citric acid salt of the title compound. This was triturated with pH 4 phthalate buffer for 18 h. The solid was collected, washed with water and dried under high vacuum yielding 0.92 g (63%) of the title compound as a white solid.

### Step D: [2-(2,4-Difluoro-phenyl)-thiazol-4-yl]-carbamic acid tert-butyl ester

2-(2,4-Difluoro-phenyl)-thiazole-4-carboxylic acid (670 mg, 2.78 mmol), diphenylphosphoryl azide (801 mg, 2.91 mmol), and triethylamine (295 mg, 2.91 mmol) were refluxed in t-butanol (5 mL) for 4 h. The reaction mixture was concentrated and the residue chromatographed yielding 554 mg (64%) of the title compound.

### Step E: 2-(2,4-Difluoro-phenyl)-thiazol-4-ylamine

[2-(2,4-Difluoro-phenyl)-thiazol-4-yl]-carbamic acid tert-butyl ester (540 mg, 1.73 mmol) was stirred at room temperature for 90 min in a mixture of TFA (5 mL) and CH₂Cl₂ (5 mL). The reaction was concentrated yielding the title compound contaminated with ˜10% of N-[2-(2,4-difluoro-phenyl)-thiazol-4-yl]-2,2,2-trifluoro-acetamide.

### Step F: N-[2-(2,4-Difluoro-phenyl)-thiazol-4-yl]-4-(pyrimidin-4-ylamino)-benzamide

2-(2,4-Difluoro-phenyl)-thiazol-4-ylamine (≦1.73 mmol) and 4-(pyrimidin-4-ylamino)-benzoyl chloride hydrochloride (424 mg, 1.57 mmol/from Preparation A) were heated in pyridine (4 mL) at 80° C. for 18 h. The reaction mixture was concentrated and the residue chromatographed yielding 151 mg (22%) of the title compound as an off-white solid. LC-MS m/z (M+H)⁺ 410, (M−H)⁻ 408.

### Example 4

### N-[2-(2-Fluoro-3-trifluoromethyl-Phenyl)-thiazol-4-yl]-4-(Pyrimidin-4-ylamino)-benzamide

Utilizing the same sequence of reactions as described in Example 3 and starting with 2-fluoro-3-trifluoromethyl-benzamide (in Step A), the title compound was prepared as a yellow solid. LC-MS m/z (M+H)⁺ 460, (M−H)⁻ 458.

### Example 5

### N-[2-(4-Fluoro-3-trifluoromethyl-phenyl)-thiazol-4-yl]-4-(pyrimidin-4-ylamino)-benzamide

Utilizing the same sequence of reactions as described in Example 3 and starting with 4-fluoro-3-trifluoromethyl-benzamide (in Step A), the title compound was prepared as a light brown solid. LC-MS m/z (M+H)⁺ 460, (M−H)⁻ 458.

### Example 6

### N-[3-(4-Fluoro-3-trifluoromethyl-phenyl)-[1,2,4]thiadiazol-5-yl]-4-(Pyrimidin-4-ylamino)-benzamide

### Step A: 4-Fluoro-3-trifluoromethyl-benzamidine

Utilizing the procedure of Thurkauf et al. (J. Med. Chem. 1995, 38, 2251) 4-fluoro-3-trifluoromethyl-benzonitrile (1.89 g, 10 mmol) was converted into 1.30 g (63%) of the title compound as brown oil.

### Step B: 3-(4-Fluoro-3-trifluoromethyl-phenyl)-[1,2,4]thiadiazol-5-ylamine

Utilizing the procedure of Goerdeler et al. (Chem. Ber. 1954, 87, 57) 4-fluoro-3-trifluoromethyl-benzamidine (1.03 g, 5 mmol) was converted into 330 mg (25%) of the title compound as a crystalline solid.

### Step C: N-[3-(4-Fluoro-3-trifluoromethyl-phenyl)-[1,2,4]-thiadiazol-5-yl]-4-(pyrimidin-4-ylamino)-benzamide

Utilizing the same procedure as described in Example 3, Step F, 3-(4-fluoro-3-trifluoromethyl-phenyl)-[1,2,4]thiadiazol-5-ylamine (263 mg, 1.0 mmol) was converted into 57 mg (12%) of the title compound as a slightly yellow solid. LC-MS m/z (M+H)⁺ 461, (M−H)⁻ 459.

### Example 7

### N-[3-(2,4-Difluoro-phenyl)-[1,2,4]thiadiazol-5-yl]-4-(Pyrimidin-4-ylamino)-benzamide

Utilizing the same sequence of reactions as described in Example 6 and starting with 2,4-difluoro-benzonitrile (in Step A), the title compound was prepared as a yellowish solid. LC-MS m/z (M+H)⁺ 411, (M−H)⁻ 409.

### Example 8

### N-[3-(2-Fluoro-3-trifluoromethyl-phenyl)-[1,2,4]-thiadiazol-5-yl]-4-(pyrimidin-4-ylamino)-benzamide

Utilizing the same sequence of reactions as described in Example 6 and starting with 2-fluoro-3-trifluoromethyl-benzonitrile (in Step A), the title compound was prepared as a tan solid. LC-MS m/z (M+H)⁺ 461, (M−H)⁻ 459.

### Example 9

### N-(3-Phenyl-[1,2,4]thiadiazol-5-yl)-4-(Pyrimidin-4-ylamino)-benzamide

Utilizing the same procedure as described in Example 3, Step F, 3-phenyl-[1,2,4]thiadiazol-5-ylamine was converted into the title compound as a white solid. LC-MS m/z (M+H)⁺ 375, (M−H)⁻ 373.

### Example 10

### 4-(6-Azetidin-1-yl-pyrimidin-4-ylamino)-N-[3-(4-fluoro-3-trifluoromethyl-phenyl)-[1,2,4]thiadiazol-5-yl]-benzamide

To a suspension of 3-(4-fluoro-3-trifluoromethyl-phenyl)-[1,2,4]thiadiazol-5-ylamine (149 mg, 0.57 mmol/from Example 6, Step B) in CH₂Cl₂ (3.0 mL) was added trimethylaluminum (0.934 mL of 2.0 M solution in toluene, 1.87 mmol). The resulting solution was added to a microwave tube containing 4-(6-azetidin-1-yl-pyrimidin-4-ylamino)-benzoic acid methyl ester (161 mg, 0.57 mmol/from Preparation B) and the mixture microwaved for 30 min with a maximum temperature of 120° C. The cooled reaction mixture was transferred to a larger flask and triturated with 1.0 M Rochelle salt solution for 18 h. The mixture was filtered and the solids triturated with CH₂Cl₂/methanol. Collection and drying yielded 205 mg (70%) of the title compound as an off-white solid. LC-MS m/z (M+H)⁺ 516, (M−H)⁻ 514.

### Example 11

### 4-(6-Dimethylamino-pyrimidin-4-ylamino)-N-[3-(4-fluoro-3-trifluoromethyl-phenyl)-[1,2,4]thiadiazol-5-yl]-benzamide

Utilizing the same procedure as described in Example 10, 3-(4-fluoro-3-trifluoromethyl-phenyl)-[1,2,4]thiadiazol-5-ylamine (162 mg, 0.61 mmol/from Example 6, Step B) and 4-(6-dimethylamino-pyrimidin-4-ylamino)-benzoic acid methyl ester (167 mg, 0.61 mmol/from Preparation C) were converted into 140 mg (46%) of the title compound as a white solid. LC-MS m/z (M+H)⁺ 504, (M−H)⁻ 502.

### Example 12

### N-[3-(3-Trifluoromethoxy-phenyl)-[1,2,4]thiadiazol-5-yl]-4-(pyrimidin-4-ylamino)-benzamide

### Step A: [3-(3-Trifluoromethoxy-phenyl)-[1,2,4]thiadiazol-5-yl]-(4-methoxy-benzyl)-carbamic acid tert-butyl ester

(3-Dimethylcarbamoylmethylsulfanyl-[1,2,4]thiadiazol-5-yl)-(4-methoxy-benzyl)-carbamic acid tert-butyl ester (551 mg, 1.25 mmol/from Preparation D), 3-trifluoromethoxybenzene boronic acid (386 mg, 1.87 mmol), Cu(I) thiophenecarboxylate (357 mg, 1.87 mmol) and palladium bis(tri-t-butylphosphine) (64 mg, 0.125 mmol) were combined in a dry flask under N₂. To this was added by syringe dry THF (12 mL) and the mixture heated at 60° C. for 12 h. The cooled mixture was filtered rinsing with THF. The filtrate was concentrated and the residue chromatographed yielding 406 mg (67%) of the title compound as a thick oil.

### Step B: [3-(3-Trifluoromethoxy-phenyl)-[1,2,4]-thiadiazol-5-yl]-carbamic acid tert-butyl ester

[3-(3-Trifluoromethoxy-phenyl)-[1,2,4]thiadiazol-5-yl]-(4-methoxy-benzyl)-carbamic acid tert-butyl ester (403 mg, 0.84 mmol) was dissolved in acetonitrile (20 mL). To this was added water (5 mL) and ceric ammonium nitrate (1.83 g, 3.34 mmol). After stirring for 18 h at room temperature the mixture was diluted with ethyl acetate and washed with water. The organic layer was dried over MgSO₄, filtered and concentrated. The residue was dissolved in THF (20 mL) and treated with polymer bound-SO₂NHNH₂ (4.2 meq). After stirring for 2 h, the resin was filtered off and rinsed with THF. The filtrate was concentrated yielding 322 mg (100%) of the title compound as a yellow gum.

### Step C: 3-(3-Trifluoromethoxy-phenyl)-[1,2,4]thiadiazol-5-ylamine

[3-(3-Trifluoromethoxy-phenyl)-[1,2,4]thiadiazol-5-yl]-carbamic acid tert-butyl ester (≦0.84 mmole) was dissolved in CH₂Cl₂ (5 mL) and treated with TFA (5 mL) at room temperature for 2 h. The reaction was then concentrated and the residue taken up in ethyl acetate. The organic solution was washed with saturated sodium bicarbonate solution, dried over MgSO₄, filtered and concentrated yielding 208 mg (95%) of the title compound as a yellow solid.

### Step D: N-[3-(3-Trifluoromethoxy-phenyl)-[1,2,4]thiadiazol-5-yl]-4-(pyrimidin-4-ylamino)-benzamide

Utilizing the same procedure as described in Example 10, 3-(3-trifluoromethoxy-phenyl)-[1,2,4]thiadiazol-5-ylamine (200 mg, 0.77 mmol) and 4-(pyrimidin-4-ylamino)benzoic acid methyl ester (176 mg, 0.77 mmol/from Preparation E) were converted into the title compound 80 mg-(23%) as an off-white solid. LC-MS m/z (M+H)⁺ 459, (M−H)⁻ 457.

### Example 13

### N-[3-(3,4-Dichloro-phenyl)-[1,2,4]thiadiazol-5-yl]-4-(pyrimidin-4-ylamino)-benzamide

Utilizing the same sequence of reactions as described in Example 12 and starting with 3,4-dichlorobenzene boronic acid (in Step A), the title compound was prepared as a off-white solid. LC-MS m/z (M+H)⁺ 443, (M−H)⁻ 441.

### Example 14

### N-[3-(3-Fluoro-4-trifluoromethyl-phenyl)-[1,2,4]thiadiazol-5-yl]-4-(pyrimidin-4-ylamino)-benzamide

Utilizing the same sequence of reactions as described in Example 12 and starting with 3-fluoro-4-trifluoromethylbenzene boronic acid (in Step A), the title compound was prepared as a white solid. LC-MS m/z (M+H)⁺ 461, (M−H)⁻ 459.

### Example 15

### N-[3-(3,4-Difluoro-phenyl)-[1,2,4]thiadiazol-5-yl]-4-(pyrimidin-4-ylamino)-benzamide

### Step A: [3-(3,4-Difluoro-phenyl)-[1,2,4]thiadiazol-5-yl]-(4-methoxy-benzyl)-carbamic acid tert-butyl ester

Using the same procedure as described in Example 12, Step A, (3-Methylsulfanyl-[1,2,4]thiadiazol-5-yl)-(4-methoxy-benzyl)-carbamic acid tert-butyl ester (1.29 g, 3.5 mmol/from Preparation E) and 3,4-difluorobenzene boronic acid (665 mg, 4.2 mmol) were converted into 411 mg (27%) of the title compound as a thick gum.

### Step B: 3-(3,4-Difluoro-phenyl)-[1,2,4]-thiadiazol-5-ylamine

[3-(3,4-Difluoro-phenyl)-[1,2,4]thiadiazol-5-yl]-(4-methoxy-benzyl)-carbamic acid tert-butyl ester (479 mg, 1.1 mmol) was refluxed in neat TFA (10 mL) for 6 h. The cooled mixture was concentrated to a gum which was dissolved in THF and treated with solid K₂CO₃. After filtration and concentration, the residue was chromatographed yielding 126 mg (54%) of the title compound as a white solid.

### Step C: N-[3-(3,4-Difluoro-phenyl)-[1,2,4]thiadiazol-5-yl]-4-iodo-benzamide

3-(3,4-Difluoro-phenyl)-[1,2,4]thiadiazol-5-ylamine (125 mg, 0.59 mmol), 4-iodobenzoyl chloride (469 mg, 1.76 mmol) and dimethylaminopyridine (10 mg) were heated in pyridine (5 mL) at 60° C. for 48 h. The cooled mixture was concentrated and the residue dissolved in ethyl acetate. The organic solution was washed with 1N HCl and saturated sodium bicarbonate solution and dried over MgSO₄. Filtration, concentration, and chromatography yielded 136 mg (52%) of the title compound as a white solid.

### Step D: N-[3-(3,4-Difluoro-phenyl)-[1,2,4]thiadiazol-5-yl]-4-(pyrimidin-4-ylamino)-benzamide

Utilizing the same procedure as described in Example 1, Step F, N-[3-(3,4-Difluoro-phenyl)-[1,2,4]thiadiazol-5-yl]-4-iodo-benzamide (136 mg, 0.31 mmol) was converted into 36 mg (29%) of the title compound as a yellow solid. LC-MS m/z (M+H)⁺ 411, (M−H)⁻ 409.

### Example 16

### N-[5-(4-Fluoro-3-trifluoromethyl-phenyl)-[1,2,4]thiadiazol-3-yl]-4-(pyridazin-4-ylamino)-benzamide

### Step A: N-[5-(4-Fluoro-3-trifluoromethyl-phenyl)-[1,2,4]thiadiazol-3-yl]-4-iodo-benzamide

Utilizing the same procedure as described in Example 15, Step C, 3-(4-fluoro-3-trifluoromethyl-phenyl)-[1,2,4]thiadiazol-5-ylamine (1.31 g, 5.0 mmol/from Example 6, Step B) and 4-iodobenzoyl chloride (2.66 g, 10.0 mmol) were converted into 927 mg (38%) of the title compound as a white powder.

### Step B: N-[5-(4-Fluoro-3-trifluoromethyl-phenyl)-[1,2,4]thiadiazol-3-yl]-4-(pyridazin-4-ylamino)-benzamide

Utilizing the same procedure as described in Example 1, Step F, N-[5-(4-fluoro-3-trifluoromethyl-phenyl)-[1,2,4]thiadiazol-3-yl]-4-iodo-benzamide (370 mg, 0.75 mmol) was converted into 15 mg (4%) of the title compound. LC-MS m/z (M+H)⁺ 461, (M−H)⁻ 459.

### Example 17

### N-[5-(3,4-Difluoro-phenyl)-[1,2,4]thiadiazol-3-yl]-4-(pyrimidin-4-ylamino)-benzamide

### Step A: N′-[5-(3,4-Difluoro-phenyl)-[1,2,4]thiadiazol-3-yl]-N,N-dimethyl-formamidine

N,N-Dimethyl-N′-(5-methylsulfanyl-[1,2,4]thiadiazol-3-yl)-formamidine (606 mg, 3.0 mmol/from Preparation G), 3,4-difluorobenzene boronic acid (568 mg, 3.6 mmol), Cu (I) thiophene carboxylate (858 mg, 4.5 mmol), zinc acetate (550 mg, 3.0 mmol) and palladium bis(tri-t-butylphosphine) (307 mg, 0.6 mmol) were combined in a dry flask under N₂. THF (30 mL) was added by syringe and the mixture heated at 60° C. for 20 h. The cooled mixture was diluted with ethyl acetate and filtered. The filtrate was washed with saturated sodium bicarbonate solution and dried over MgSO₄. Filtration and concentration gave an oil which was chromatographed yielding 291 mg (36%) of the title compound as a yellow solid.

### Step B: 5-(3,4-Difluoro-phenyl)-[1,2,4]-thiadiazol-3-ylamine

N′-[5-(3,4-Difluoro-phenyl)-[1,2,4]thiadiazol-3-yl]-N,N-dimethyl-formamidine (285 mg, 1.06 mmol) and p-toluenesulphonic acid (404 mg, 2.12 mmol) were heated together in methanol (20 mL) for 20 h. The mixture was concentrated and the residue taken up in ethyl acetate. The organic solution was washed with saturated sodium bicarbonate solution, dried over MgSO₄, filtered, and concentrated yielding 226 mg (100%) of the title compound as an off-white solid.

### Step C: N-[5-(3,4-Difluoro-phenyl)-[1,2,4]-thiadiazol-3-yl]-4-(pyrimidin-4-ylamino)-benzamide

Utilizing the same procedure as described in Example 10, 5-(3,4-difluoro-phenyl)-[1,2,4]thiadiazol-3-ylamine (250 mg, 1.17 mmol) and 4-(pyrimidin-4-ylamino)benzoic acid methyl ester (268 mg, 1.17 mmol/from Preparation E) were converted into 16 mg (3%) of the title compound. LC-MS m/z (M+H)⁺ 411, (M−H)⁻ 409.

### Example 18

### N-[4-(2-Fluoro-3-trifluoromethyl-phenyl)-thiazol-2-yl]-4-(pyrimidin-4-ylamino)-benzamide

### Step A: 2-Chloro-1-(2-fluoro-3-trifluoromethyl-phenyl)-ethanone

1-(2-Fluoro-3-trifluoromethyl-phenyl)-ethanone (15.0 g, 72.77 mmol) and sulfuryl chloride (20.0 g, 148.2 mmol) were combined and heated to 50° C. for 45 min. The reaction mixture was concentrated yielding the title compound as a clear colorless oil.

### Step B: 4-(2-Fluoro-3-trifluoromethyl-phenyl)-thiazol-2-ylamine

2-Chloro-1-(2-fluoro-3-trifluoromethyl-phenyl)-ethanone (17.5 g, 72.77 mmol) and thiourea (5.7 g, 75.0 mmol) were refluxed together in ethanol (150 mL) for 18 h. The reaction mixture was concentrated and the residue taken up in CH₂Cl₂ and saturated sodium bicarbonate solution. The separated organic layer was washed with saturated sodium bicarbonate solution, dried over MgSO₄, filtered and concentrated to a white solid. This was triturated with hexane and collected yielding 11.74 g (62%) of the title compound as white needles.

### Step C: N-[4-(2-Fluoro-3-trifluoromethyl-phenyl)-thiazol-2-yl]-4-(pyrimidin-4-ylamino)-benzamide

Utilizing the same procedure as described in Example 3, Step F, 5-(2-fluoro-3-trifluoromethyl-phenyl)-thiazol-2-ylamine (1.49 g, 5.5 mmol) was converted into 1.35 g (59%) of the title compound as a white solid. LC-MS m/z (M+H)⁺ 460, (M−H)⁻ 458.

### Examples 19-50

Utilizing the same sequence of reactions as described in Example 18 and starting with the corresponding 1-phenylethanone, 2-halo-1-phenyl-ethanone, or N-(4-aryl)-thiazol-2-ylamine depending on commercial availability of the materials, the following examples were prepared:

### Example 19

### N-[4-(Phenyl)-thiazol-2-yl]-4-(pyrimidin-4-ylamino)-benzamide

LC-MS m/z (M+H)⁺ 374, (M−H)⁻ 372.

### Example 20

### N-[4-(2-Fluoro-Phenyl)-thiazol-2-yl]-4-(pyrimidin-4-ylamino)-benzamide

LC-MS m/z (M+H)⁺ 392, (M−H)⁻ 390.

### Example 21

### N-[4-(3-Fluoro-phenyl)-thiazol-2-yl]-4-(pyrimidin-4-ylamino)-benzamide

LC-MS m/z (M+H)⁺ 392, (M−H)⁻ 390.

### Example 22

### N-[4-(4-Fluoro-phenyl)-thiazol-2-yl]-4-(pyrimidin-4-ylamino)-benzamide

LC-MS m/z (M+H)⁺ 392, (M−H)⁻ 390.

### Example 23

### N-[4-(2,3-Difluoro-phenyl)-thiazol-2-yl]-4-(pyrimidin-4-ylamino)-benzamide

LC-MS m/z (M+H)⁺ 410, (M−H)⁻ 408.

### Example 24

### N-[4-(2,4-Difluoro-phenyl)-thiazol-2-yl]-4-(pyrimidin-4-ylamino)-benzamide

LC-MS m/z (M+H)⁺ 410, (M−H)⁻ 408.

### Example 25

### N-[4-(2,6-Difluoro-phenyl)-thiazol-2-yl]-4-(pyrimidin-4-ylamino)-benzamide

LC-MS m/z (M+H)⁺ 410, (M−H)⁻ 408.

### Example 26

### N-[4-(3,4-Difluoro-Phenyl)-thiazol-2-yl]-4-(pyrimidin-4-ylamino)-benzamide

LC-MS m/z (M+H)⁺ 410, (M−H)⁻ 408.

### Example 27

### N-[4-(2-Chloro-phenyl)-thiazol-2-yl]-4-(pyrimidin-4-ylamino)-benzamide

LC-MS m/z (M+H)⁺ 408, (M−H)⁻ 406.

### Example 28

### N-[4-(3-Chloro-phenyl)-thiazol-2-yl]-4-(pyrimidin-4-ylamino)-benzamide

LC-MS m/z (M+H)⁺ 408, (M−H)⁻ 406.

### Example 29

### N-[4-(4-Chloro-phenyl)-thiazol-2-yl]-4-(pyrimidin-4-ylamino)-benzamide

LC-MS m/z (M+H)⁺ 408, (M−H)⁻ 406.

### Example 30

### N-[4-(2,3-Dichloro-phenyl)-thiazol-2-yl]-4-(pyrimidin-4-ylamino)-benzamide

LC-MS m/z (M+H)⁺ 442, (M−H)⁻ 440.

### Example 31

### N-[4-(2,4-Dichloro-Phenyl)-thiazol-2-yl]-4-(pyrimidin-4-ylamino)-benzamide

LC-MS m/z (M+H)⁺ 442, (M−H)⁻ 440.

### Example 32

### N-[4-(3,4-Dichloro-phenyl)-thiazol-2-yl]-4-(pyrimidin-4-ylamino)-benzamide

LC-MS m/z (M+H)⁺ 442, (M−H)⁻ 440.

### Example 33

### N-[4-(2-Trifluoromethyl-phenyl)-thiazol-2-yl]-4-(pyrimidin-4-ylamino)-benzamide

LC-MS m/z (M+H)⁺ 442, (M−H)⁻ 440.

### Example 34

### N-[4-(3-Trifluoromethyl-phenyl)-thiazol-2-yl]-4-(pyrimidin-4-ylamino)-benzamide

LC-MS m/z (M+H)⁺ 442, (M−H)⁻ 441.

### Example 35

### N-[4-(4-Trifluoromethyl-phenyl)-thiazol-2-yl]-4-(pyrimidin-4-ylamino)-benzamide

LC-MS m/z (M+H)⁺ 442, (M−H)⁻ 440.

### Example 36

### N-[4-(2-Fluoro-3-chloro-phenyl)-thiazol-2-yl]-4-(pyrimidin-4-ylamino)-benzamide

### Example 37

### N-[4-(2-Fluoro-4-chloro-phenyl)-thiazol-2-yl]-4-(pyrimidin-4-ylamino)-benzamide

LC-MS m/z (M+H)⁺ 426, (M−H)⁻ 429.

### Example 38

### N-[4-(2-Fluoro-4-trifluoromethyl-phenyl)-thiazol-2-yl]-4-(pyrimidin-4-ylamino)-benzamide

LC-MS m/z (M+H)⁺ 460, (M−H)⁻ 458.

### Example 39

### N-[4-(2-Fluoro-5-trifluoromethyl-phenyl)-thiazol-2-yl]-4-(pyrimidin-4-ylamino)-benzamide

LC-MS m/z (M+H)⁺ 460, (M−H)⁻ 458.

### Example 40

### N-[4-(4-Fluoro-3-trifluoromethyl-phenyl)-thiazol-2-yl]-4-(pyrimidin-4-ylamino)-benzamide

LC-MS m/z (M+H)⁺ 460, (M−H)⁻ 458.

### Example 41

### N-[4-(3-Fluoro-4-trifluoromethyl-phenyl)-thiazol-2-yl]-4-(pyrimidin-4-ylamino)-benzamide

LC-MS m/z (M+H)⁺ 460, (M−H)⁻ 458.

### Example 42

### N-[4-(2,6-Dichloro-4-trifluoromethyl-phenyl)-thiazol-2-yl]-4-(pyrimidin-4-ylamino)-benzamide

LC-MS m/z (M+H)⁺ 510, (M−H)⁻ 508.

### Example 43

### N-[4-(4-Methyl-phenyl)-thiazol-2-yl]-4-(pyrimidin-4-ylamino)-benzamide

LC-MS m/z (M+H)⁺ 388, (M−H)⁻ 386.

### Example 44

### N-[4-(2,4-Dimethyl-phenyl)-thiazol-2-yl]-4-(pyrimidin-4-ylamino)-benzamide

LC-MS m/z (M+H)⁺ 402, (M−H)⁻ 400.

### Example 45

### N-[4-(4-Difluoromethoxy-phenyl)-thiazol-2-yl]-4-(pyrimidin-4-ylamino)-benzamide

LC-MS m/z (M+H)⁺ 440, (M−H)⁻ 438.

### Example 46

### N-[4-(3-Trifluoromethoxy-phenyl)-thiazol-2-yl]-4-(pyrimidin-4-ylamino)-benzamide

LC-MS m/z (M+H)⁺ 458, (M−H)⁻ 456.

### Example 47

### N-[4-(4-Trifluoromethoxy-phenyl)-thiazol-2-yl]-4-(pyrimidin-4-ylamino)-benzamide

LC-MS m/z (M+H)⁺ 458, (M−H)⁻ 456.

### Example 48

### N-[4-(2-Naphthyl)-thiazol-2-yl]-4-(pyrimidin-4-ylamino)-benzamide

LC-MS m/z (M+H)⁺ 424, (M−H)⁻ 422.

### Example 49

### 4-(Pyrimidin-4-ylamino)-N-[4-(3-bromo-phenyl)-thiazol-2-yl]-benzamide

LC-MS m/z (M+H)⁺ 454, (M−H)⁻ 452.

### Example 50

### 4-(Pyrimidin-4-ylamino)-N-[4-(2-fluoro-3-bromo-phenyl)-thiazol-2-yl]-benzamide

LC-MS m/z (M+H)⁺ 472, (M−H)⁻ 470.

### Example 51

### 4-(Pyrimidin-4-ylamino)-N-[4-(2,3,4-trifluoro-phenyl)-thiazol-2-yl]-benzamide

### Step A: (4-Methoxy-benzyl)-[4-(2,3,4-trifluoro-phenyl)-thiazol-2-yl]-carbamic acid tert-butyl ester

Trifluoro-methanesulfonic acid 2-[tert-butoxycarbonyl-(4-methoxy-benzyl)-amino]-thiazol-4-yl ester (408 mg, 0.87 mmol/from Preparation H), 4,4,5,5-tetramethyl-2-(2,3,4-trifluoro-phenyl)-[1,3,2]dioxaborolane (270 mg, 1.04 mmol), cesium carbonate (567 mg, 1.74 mmol), tetrakis(triphenylphosphine)palladium (104 mg, 0.09 mmol) and powdered 4 Å molecular sieves were combined in a dry flask that was then purged with nitrogen. Dry, nitrogen purged dioxane (9 mL) was added by syringe and the mixture heated to 100° C. for 3 h. The cooled mixture was filtered through diatomaceous earth rinsing with THF. The filtrate was concentrated to an oil which was chromatographed yielding 345 mg of the title compound as a thick gum.

### Step B: 4-(2,3,4-Trifluoro-phenyl)-thiazol-2-ylamine

(4-Methoxy-benzyl)-[4-(2,3,4-trifluoro-phenyl)-thiazol-2-yl]-carbamic acid tert-butyl ester (340 mg, 0.75 mmol) was dissolved in neat trifluoroacetic acid (5 mL) and heated to reflux for 5 h. The cooled mixture was concentrated to a gum which was redissolved in THF and reconcentrated. The residue was chromatographed yielding 171 mg (99%) of the title compound as a white solid.

### Step C: 4-(Pyrimidin-4-ylamino)-N-[4-(2,3,4-trifluoro-phenyl)-thiazol-2-yl]-benzamide

Utilizing the same procedure as described in Example 3, Step F, 4-(2,3,4-trifluoro-phenyl)-thiazol-2-ylamine (161 mg, 0.70 mmol) was converted into 85 mg (28%) of the title compound as solid. LC-MS m/z (M+H)⁺ 428, (M−H)⁻ 426.

### Example 52

### 4-(Pyrimidin-4-ylamino)-N-[4-(2,3-difluoro-4-trifluoromethyl-phenyl)-thiazol-2-yl]-benzamide

Utilizing the same sequence of reactions as described in Example 51 and starting with 4,4,5,5-tetramethyl-2-(2,3-difluoro-4-trifluoromethyl-phenyl)-[1,3,2]dioxaborolane (in Step A), the title compound was prepared as a solid. LC-MS m/z (M+H)⁺ 472, (M−H)⁻ 470.

### Example 53

### 4-(Pyrimidin-4-ylamino)-N-[4-(6-trifluoromethyl-pyridin-2-yl)-thiazol-2-yl]-benzamide

Utilizing the same sequence of reactions as described in Example 51 and starting with 4,4,5,5-tetramethyl-2-(6-trifluoromethyl-pyridin-2-yl)-[1,3,2]dioxaborolane (in Step A), the title compound was prepared as a yellow solid. LC-MS m/z (M+H)⁺ 443, (M−H)⁻ 441.

### Example 54

### 4-(Pyrimidin-4-ylamino)-N-[4-(2-fluoro-3-trifluoromethoxy-phenyl)-thiazol-2-yl]-benzamide

Utilizing the same sequence of reactions as described in Example 51, Step A, Example 15, Step C and Example 1, Step F and starting with 4,4,5,5-tetramethyl-2-(2-fluoro-3-trifluoromethoxy-phenyl)-[1,3,2]dioxaborolane (in Example 51, Step A), the title compound was prepared as a yellow solid. LC-MS m/z (M+H)⁺ 476, (M−H)⁻ 474.

### Example 55

### 4-(Pyridin-2-ylamino)-N-[4-(2-fluoro-3-trifluoromethyl-phenyl)-thiazol-2-yl]-benzamide

### Step A: 4-(Pyridin-2-ylamino)-benzoic acid tert-butyl ester

Utilizing the same procedure as described in Example 1, Step F, 4-bromobenzoic acid tert-butyl ester (515 mg, 2.0 mmol) and pyridin-2-ylamine (236 mg, 2.4 mmol) were converted into 180 mg (33%) of the title compound as a white solid.

### Step B: 4-(Pyridin-2-ylamino)-benzoic acid

Utilizing the same procedure as described in Example 12, Step C, 4-(pyridin-2-ylamino)-benzoic acid tert-butyl ester (173 mg, 0.64 mmol) was converted into the title compound as a white solid.

### Step C: 4-(Pyridin-2-ylamino)-benzoyl chloride

Utilizing the same procedure as described in Procedure A, Step 3,4-(pyridin-2-ylamino)-benzoic acid (≦0.64 mmol) was converted into 89 mg (52%) of the title compound as a white solid.

### Step D: 4-(Pyridin-2-ylamino)-N-[4-(2-fluoro-3-trifluoromethyl-phenyl)-thiazol-2-yl]-benzamide

Utilizing the same procedure as described in Example 3, Step F, 4-(pyridin-2-ylamino)-benzoyl chloride (89 mg, 0.33 mmol) and 4-(2-fluoro-3-trifluoromethyl-phenyl)-thiazol-2-ylamine (87 mg, 0.33 mmol/from Example 18, Step B) were converted into 42 mg (28%) of the title compound as a yellow solid. LC-MS m/z (M+H)⁺ 459, (M−H)⁻ 457.

### Example 56

### 4-(Pyridin-3-ylamino)-N-[4-(2-fluoro-3-trifluoromethyl-phenyl)-thiazol-2-yl]-benzamide

Utilizing the same sequence of reactions as described in Example 55 and starting with pyridin-3-ylamine (in Step A), the title compound was prepared as a tan solid. LC-MS m/z (M+H)⁺ 459, (M−H)⁻ 457.

### Example 57

### 4-(Pyridin-4-ylamino)-N-[4-(2-fluoro-3-trifluoromethyl-phenyl)-thiazol-2-yl]-benzamide

Utilizing the same sequence of reactions as described in Example 55 and starting with pyridin-4-ylamine (in Step A), the title compound was prepared as a tan solid. LC-MS m/z (M+H)⁺ 459, (M−H)⁻ 457.

### Example 58

### 4-(Pyridazin-4-ylamino)-N-[4-(2-fluoro-3-trifluoromethyl-phenyl)-thiazol-2-yl]-benzamide

Utilizing the same sequence of reactions as described in Example 55 and starting with pyridazin-4-ylamine (in Step A), the title compound was prepared as a solid. LC-MS m/z (M+H)⁺ 460, (M−H)⁻ 458.

### Example 59

### 4-(Pyridazin-4-ylamino)-N-[4-(4-chloro-Phenyl)-thiazol-2-yl]-benzamide

Utilizing the same sequence of reactions as described in Example 55 and starting with pyridazin-4-ylamine (in Step A) and 4-(4-chloro-phenyl)-thiazol-2-ylamine (in Step D), the title compound was prepared as an off-white solid. LC-MS m/z (M+H)⁺ 408, (M−H)⁻ 406.

### Example 60

### 4-(Pyridazin-4-ylamino)-N-[4-(2,4-difluoro-phenyl)-thiazol-2-yl]-benzamide

Utilizing the same sequence of reactions as described in Example 55 and starting with pyridazin-4-ylamine (in Step A) and 4-(2,4-difluoro-phenyl)-thiazol-2-ylamine (in Step D), the title compound was prepared as a solid. LC-MS m/z (M+H)⁺ 410, (M−H)⁻ 408.

### Example 61

### 4-(Pyridazin-4-ylamino)-N-[4-(4-fluoro-3-trifluoromethyl-phenyl)-thiazol-2-yl]-benzamide

Utilizing the same sequence of reactions as described in Example 55 and starting with pyridazin-4-ylamine (in Step A) and 4-(4-fluoro-3-trifluoromethyl-phenyl)-thiazol-2-ylamine (in Step D), the title compound was prepared as an off-white solid. LC-MS m/z (M+H)⁺ 460, (M−H)⁻ 458.

### Example 62

### 4-(Pyrazin-2-ylamino)-N-[4-(2-fluoro-3-trifluoromethyl-phenyl)-thiazol-2-yl]-benzamide

### Step A: 4-(Pyrazin-2-ylamino)-benzoic acid tert-butyl ester

Pyrazin-2-ylamine (475 mg, 5.0 mmol), 4-fluorobenzoic acid tert-butyl ester (981 mg, 5.0 mmol) and potassium tert-butoxide (6.0 mL at 1.0 M in THF, 6.0 mmol) were combined in dry DMF (5 mL) and heated to 80° C. for 18 h. The cooled reaction mixture was diluted with ethyl acetate and washed with water. The organic layer was dried over MgSO₄, filtered, and concentrated. The residue was triturated with ether/hexane. The solid by-product was filtered off and the filtrate concentrated and the residue chromatographed yielding 252 mg (17%, adjusted for presence of side product) of the title compound as a yellow solid contaminated with ˜20% of 4-fluoro-N-pyrazin-2-yl-benzamide.

### Step B: 4-(Pyrazin-2-ylamino)-benzoic acid

Utilizing the same procedure as described in Example 12, Step C, 4-(pyrazin-2-ylamino)-benzoic acid tert-butyl ester (238 mg, 0.88 mmol) was converted into the title compound as a white solid.

### Step C: 4-(Pyrazin-2-ylamino)-benzoyl chloride

Utilizing the same procedure as described in Preparation A, Step 3,4-(pyrazin-2-ylamino)-benzoic acid (s 0.88 mmol) was converted into the title compound as a yellow solid.

### Step D: 4-(Pyrazin-2-ylamino)-N-[4-(2-fluoro-3-trifluoromethyl-phenyl)-thiazol-2-yl]-benzamide

Utilizing the same procedure as described in Example 3, Step F, 4-(pyrazin-2-ylamino)-benzoyl chloride (s 0.88 mmol) and 4-(2-fluoro-3-trifluoromethyl-phenyl)-thiazol-2-ylamine (231 mg, 0.88 mmol/from Example 18, Step B) were converted into 42 mg (10%) of the title compound as a yellow solid. LC-MS m/z (M+H)⁺ 460, (M−H)⁻ 458.

### Example 63

### 4-(Pyridazin-4-ylamino)-N-[4-(4-fluoro-3-trifluoromethyl-Phenyl)-thiazol-2-yl]-benzamide

Utilizing the same sequence of reactions as described in Example 62 and starting with pyrimidin-2-ylamine (in Step A), the title compound was prepared as a yellow solid. LC-MS m/z (M+H)⁺ 460, (M−H)⁻ 458.

### Example 64

### 4-(1,3,5-Triazin-2-ylamino)-N-[4-(4-fluoro-3-trifluoromethyl-phenyl)-thiazol-2-yl]-benzamide

Utilizing the same sequence of reactions as described in Example 62 and starting with 1,3,5-triazin-2-ylamine (in Step A), the title compound was prepared as an off-white solid. LC-MS m/z (M+H)⁺ 461, (M−H)⁻ 459.

### Example 65

### N-[4-(2-Fluoro-3-trifluoromethyl-Phenyl)-thiazol-2-yl]-4-(pyrimidin-5-ylamino)-benzamide

### Step A: 4-(Pyrimidin-5-ylamino)-benzoic acid tert-butyl ester

Utilizing the same procedure as described in Example 1, Step F, 4-aminobenzoic acid tert-butyl ester (440 mg, 2.27 mmol) and 5-bromopyrimidine (302 mg, 189 mmol) were converted into 387 mg (76%) of the title compound as a tan solid.

### Step B: 4-(Pyrimidin-5-ylamino)-benzoic acid

Utilizing the same procedure as described in Example 12, Step C, 4-(pyrimidin-5-ylamino)-benzoic acid tert-butyl ester (370 mg, 1.37 mmol) was converted into the title compound as a yellow solid.

### Step C: 4-(Pyrimidin-5-ylamino)-benzoyl chloride

Utilizing the same procedure as described in Procedure A, Step 3,4-(pyrimidin-5-ylamino)-benzoic acid (≦1.37 mmol) was converted into the title compound.

### Step D: N-[4-(2-Fluoro-3-trifluoromethyl-phenyl)-thiazol-2-yl]-4-(pyrimidin-5-ylamino)-benzamide

Utilizing the same procedure as described in Example 3, Step F, 4-(pyrimidin-5-ylamino)-benzoyl chloride (≦1.37 mmol) and 4-(2-fluoro-3-trifluoromethyl-phenyl)-thiazol-2-ylamine (262 mg, 1.0 mmol/from Example 18, Step B) were converted into 99 mg (22%) of the title compound as a white solid. LC-MS m/z (M+H)⁺ 460, (M−H)⁻ 458.

### Example 66

### N-[4-(2-Fluoro-3-trifluoromethyl-phenyl)-thiazol-2-yl]-4-(pyridazin-3-ylamino)-benzamide

### Step A: N-[4-(2-Fluoro-3-trifluoromethyl-phenyl)-thiazol-2-yl]-4-iodo-benzamide

Utilizing the same procedure as described in Example 15, Step C, 4-(2-fluoro-3-trifluoromethyl-phenyl)-thiazol-2-ylamine (1.31 g, 5.0 mmol/from Example 18, Step B) was converted into 1.92 g (75%) of the title compound as a pale yellow solid.

### Step B: N-[4-(2-Fluoro-3-trifluoromethyl-phenyl)-thiazol-2-yl]-4-(pyridazin-3-ylamino)-benzamide

Utilizing the same procedure as described in Example 1, Step F, N-[4-(2-Fluoro-3-trifluoromethyl-phenyl)-thiazol-2-yl]-4-iodo-benzamide (492 mg, 1.0 mmol) and pyridazin-3-ylamine (115 mg, 1.2 mmol) were converted into 113 mg (25%) of the title compound as a solid. LC-MS m/z (M+H)⁺ 460, (M−H)⁻ 458.

### Example 67

### N-[4-(2-Fluoro-3-trifluoromethyl-phenyl)-thiazol-2-yl]-4-(1,3,4-triazin-3-ylamino)-benzamide

Utilizing the same sequence of reactions as described in Example 66 and starting with 1,3,4-triazin-3-ylamine (in Step B), the title compound was prepared as a yellow solid. LC-MS m/z (M+H)⁺ 461, (M−H)⁻ 459.

### Example 68

### 4-(6-Dimethylamino-pyrimidin-4-ylamino)-N-[4-(2-fluoro-3-trifluoromethyl-phenyl)-thiazol-2-yl]-benzamide

Utilizing the same procedure as described in Example 10 and starting with 4-(6-Dimethylamino-pyrimidin-4-ylamino)-benzoic acid methyl ester (from Preparation C) and 4-(2-Fluoro-3-trifluoromethyl-phenyl)-thiazol-2-ylamine (from Example 18, Step B), the title compound was prepared as a white solid. LC-MS m/z (M+H)⁺ 503, (M−H)⁻ 501.

### Example 69

### 4-(6-Azetidin-1-yl-pyrimidin-4-ylamino)-N-[4-(2-fluoro-3-trifluoromethyl-phenyl)-thiazol-2-yl]-benzamide

Utilizing the same procedure as described in Example 10 and starting with 4-(6-azetidin-1-yl-pyrimidin-4-ylamino)-benzoic acid methyl ester (from Preparation B) and 4-(2-Fluoro-3-trifluoromethyl-phenyl)-thiazol-2-ylamine (from Example 18, Step B) the title compound was prepared as a white solid. LC-MS m/z (M+H)⁺ 515, (M−H)⁻ 513.

### Example 70

### N-[4-(2-Fluoro-3-trifluoromethyl-phenyl)-thiazol-2-yl]-4-(6-pyrrolidin-1-yl-pyrimidin-4-ylamino)-benzamide

Utilizing the same sequence of reactions as described in Preparation B and Example 10 and starting with pyrrolidine (in Preparation B) and 4-(2-Fluoro-3-trifluoromethyl-phenyl)-thiazol-2-ylamine (from Example 18, Step B) (in Example 10), the title compound was prepared as a white solid. LC-MS m/z (M+H)⁺ 529, (M−H)⁻ 527.

### Example 71

### 4-(6-Dimethylamino-pyrimidin-4-ylamino)-N-[4-(2-fluoro-3-trifluoromethyl-phenyl)-thiazol-2-yl]-benzamide

Utilizing the same sequence of reactions as described in Preparation B and Example 10 and starting with morpholine (in Preparation B) and 4-(2-Fluoro-3-trifluoromethyl-phenyl)-thiazol-2-ylamine (from Example 18, Step B) (in Example 10), the title compound was prepared as a white solid. LC-MS m/z (M+H)⁺ 545, (M−H)⁻ 543.

### Example 72

### 4-(6-Chloro-pyrimidin-4-ylamino)-N-[4-(2-fluoro-3-trifluoromethyl-phenyl)-thiazol-2-yl]-benzamide

Utilizing the same sequence of reactions as described in Preparation A, Step 1, Preparation A, Step 3, and Example 3, Step F and starting with 4-(2-fluoro-3-trifluoromethyl-phenyl)-thiazol-2-ylamine (from Example 18, Step B) (in Example 3, Step F), the title compound was prepared as a solid. LC-MS m/z (M+H)⁺ 494, (M−H)⁻ 492.

### Example 73

### 4-(6-Chloro-pyrimidin-4-ylamino)-N-[4-(4-fluoro-3-trifluoromethyl-phenyl)-thiazol-2-yl]-benzamide

Utilizing the same sequence of reactions as described in Preparation A, Step 1, Preparation A, Step 3, and Example 3, Step F and starting with 4-(4-fluoro-3-trifluoromethyl-phenyl)-thiazol-2-ylamine (from Example 41) (in Example 3, Step F), the title compound was prepared as a solid. LC-MS m/z (M+H)⁺ 494, (M−H)⁻ 492.

### Example 74

### 4-(6-Ethylamino-pyrimidin-4-ylamino)-N-[4-(2-fluoro-3-trifluoromethyl-phenyl)-thiazol-2-yl]-benzamide

4-(6-Chloro-pyrimidin-4-ylamino)-N-[4-(2-fluoro-3-trifluoromethyl-phenyl)-thiazol-2-yl]-benzamide (207 mg, 0.42 mmol/from Example 72) and ethylamine (0.21 mL of 2.0M in methanol, 0.84 mmol) were heated together in N-methylpyrrolidine (5 mL) at 100° C. in a sealed tube. The cooled mixture was concentrated to a white solid which was chromatographed yielding 10 mg (5%) of the title compound as a solid. LC-MS m/z (M+H)⁺ 503, (M−H)⁻ 501.

### Example 75

### 4-(6-Cyclopropylamino-pyrimidin-4-ylamino)-N-[4-(2-fluoro-3-trifluoromethyl-phenyl)-thiazol-2-yl]-benzamide

Utilizing the same procedure as described in Example 74 and starting with cyclopropylamine, the title compound was prepared as a solid. LC-MS m/z (M+H)⁺ 515, (M−H)⁻ 513.

### Example 76

### N-[4-(2-Fluoro-3-trifluoromethyl-phenyl)-thiazol-2-yl]-4-(6-piperidin-1-yl-pyrimidin-4-ylamino)-benzamide

Utilizing the same procedure as described in Example 74 and starting with piperidine, the title compound was prepared as a solid. LC-MS m/z (M+H)⁺ 543, (M−H)⁻ 541.

### Example 77

### N-[4-(2-Fluoro-3-trifluoromethyl-phenyl)-4-thiazol-2-yl]-4-(6-methyl-pyrimidin-4-ylamino)-benzamide

Utilizing the same sequence of reactions as described in Preparation A and Example 3, Step F and starting with 2,4-dichloro-6-methylpyrimidine (in Preparation A, Step 1) and 4-(2-fluoro-3-trifluoromethyl-phenyl)-thiazol-2-ylamine (from Example 18, Step B) (in Example 3, Step F), the title compound was prepared. LC-MS m/z (M+H)⁺ 474, (M−H)⁻ 472.

### Example 78

### 4-(2,6-Dichloro-pyrimidin-4-ylamino)-N-[4-(2-fluoro-3-trifluoromethyl-phenyl)-thiazol-2-yl]-benzamide

Utilizing the same procedure as described in Example 1, Step F and starting with 4-amino-2,6-dichloropyrimidine and N-[4-(2-Fluoro-3-trifluoromethyl-phenyl)-thiazol-2-yl]-4-iodo-benzamide (from Example 66, Step A), the title compound was prepared. LC-MS m/z (M−H)⁻ 526/528.

### Example 79

### 4-(2-Chloro-6-methyl-pyrimidin-4-ylamino)-N-[4-(2-fluoro-3-trifluoromethyl-phenyl)-thiazol-2-yl]-benzamide

Utilizing the same sequence of reactions as described in Preparation A, Step 1, Preparation A, Step 3, and Example 3, Step F and starting with 2,4-dichloro-6-methylpyrimidine (in Preparation A, Step 1) and 4-(2-fluoro-3-trifluoromethyl-phenyl)-thiazol-2-ylamine (from Example 18, Step B) (in Example 3, Step F), the title compound was prepared. LC-MS m/z (M−H)⁻ 506.

### Example 80

### 4-(2,6-Dimethyl-pyrimidin-4-ylamino)-N-[4-(2-fluoro-3-trifluoromethyl-phenyl)-thiazol-2-yl]-benzamide

Utilizing the same sequence of reactions as described in Example 62, Step A, Example 3, Step E, Preparation A, Step 3, and Example 3, Step F and starting with 4-amino-2,6-dimethylpyrimidine (in Example 62, Step A) and 4-(2-fluoro-3-trifluoromethyl-phenyl)-thiazol-2-ylamine (from Example 18, Step B) (in Example 3, Step F), the title compound was prepared. LC-MS m/z (M+H)⁺ 488, (M−H)⁻ 486.

### Example 81

### 4-(2,6-Dimethyl-pyrimidin-4-ylamino)-N-[4-(3,4-difluoro-phenyl)-thiazol-2-yl]-benzamide

Utilizing the same sequence of reactions as described in Example 62, Step A, Example 3, Step E, Preparation A, Step 3, and Example 3, Step F and starting with 4-amino-2,6-dimethylpyrimidine (in Example 62, Step A) and 4-(3,4-difluoro-phenyl)-thiazol-2-ylamine (from Example 26) (in Example 3, Step F), the title compound was prepared. LC-MS m/z (M+H)⁺ 438, (M−H)⁻ 436.

### Example 82

### 4-(6-Chloropyrimidin-4-ylamino)-N-(3-(4-fluoro-3-(trifluoromethyl)phenyl)-1,2,4-thiadiazol-5-yl)benzamide

### Step A: Methyl 4-(6-chloropyrimidin-4-ylamino)benzoate

Utilizing the same procedure as described in Preparation E and starting with 4-(6-chloro-pyrimidin-4-ylamino)-benzoyl chloride hydrochloride (prepared from 4-(6-chloro-pyrimidin-4-ylamino)-benzoic acid (from Preparation A) and utilizing the procedure described in Preparation A, Step 3) the title compound was prepared. LC-MS m/z (M+H)⁺ 264, (M−H)⁻ 262.

### Step B: 4-(6-Chloropyrimidin-4-ylamino)-N-(3-(4-fluoro-3-(trifluoromethyl)phenyl)-1,2,4-thiadiazol-5-yl)benzamide

Utilizing the same procedure as described in Example 10 and starting with methyl 4-(6-chloropyrimidin-4-ylamino)benzoate and the hydrochloride salt of 3-(4-fluoro-3-trifluoromethyl-phenyl)-[1,2,4]thiadiazol-5-ylamine (prepared from the parent amine (from Example 6, Step B) and HCl gas in CH₂Cl₂) but using thermal (reflux for 18 h) rather than microwave conditions, the title compound was prepared. LC-MS m/z (M+H)⁺ 495, (M−H)⁻ 493.

### Example 83

### 4-(6-(N-(2-Methoxyethyl)-N-methylamino)pyrimidin-4-ylamino)-N-(3-(4-fluoro-3-(trifluoromethyl)phenyl)-1,2,4-thiadiazol-5-yl)benzamide

### Step A: 6-Chloro-N-(2-methoxyethyl)-N-methylpyrimidin-4-amine

Utilizing the same procedure as described in Preparation B, Step 1 and starting with 4,6-dichloropyrimidine and 2-methoxy-N-methylethanamine, the title compound was prepared. LC-MS m/z (M+H)⁺ 202.

### Step B: Methyl 4-(6-(N-(2-methoxyethyl)-N-methylamino)pyrimidin-4-ylamino)benzoate

Utilizing the same procedure as described in Example 1, Step F and starting with 6-chloro-N-(2-methoxyethyl)-N-methylpyrimidin-4-amine and methyl 4-aminobenzoate, the title compound was prepared. LC-MS m/z (M+H)⁺ 317, (M−H)⁻ 315.

### Step C: 4-(6-(N-(2-Methoxyethyl)-N-methylamino)pyrimidin-4-ylamino)-N-(3-(4-fluoro-3-(trifluoromethyl)phenyl)-1,2,4-thiadiazol-5-yl)benzamide

Utilizing the same procedure as described in Example 10 and starting with methyl 4-(6-(N-(2-methoxyethyl)-N-methylamino)pyrimidin-4-ylamino)benzoate and 3-(4-fluoro-3-trifluoromethyl-phenyl)-[1,2,4]thiadiazol-5-ylamine (from Example 6, Step B), the title compound was prepared. LC-MS m/z (M+H)⁺ 547, (M−H)⁻ 545.

### Example 84

### 4-(6-(N-(2-Methoxyethyl)-N-methylamino)pyrimidin-4-ylamino)-N-(4-(2-fluoro-3-(trifluoromethyl)phenyl)thiazol-2-yl)benzamide

Utilizing the same procedure as described in Example 10 and starting with methyl 4-(6-(N-(2-methoxyethyl)-N-methylamino)pyrimidin-4-ylamino)benzoate (from Example 83) and 4-(2-fluoro-3-trifluoromethyl-phenyl)-thiazol-2-ylamine (from Example 18, Step B), the title compound was prepared. LC-MS m/z (M+H)⁺ 547, (M−H)⁻ 545.

### Example 85

### N-(4-(2,4-Difluoro-3-(trifluoromethyl)phenyl)thiazol-2-yl)-4-(pyrimidin-4-ylamino)benzamide

Utilizing the same sequence of reactions as described in Example 51 and starting with 2-(2,4-difluoro-3-(trifluoromethyl)phenyl)-4,4,5,5-tetramethyl-1,3,2-dioxaborolane, the title compound was prepared. LC-MS m/z (M+H)⁺ 478, (M−H)⁻ 476.

### Example 86

### N-(5-(2-Fluoro-3-(trifluoromethoxy)phenyl)-1,2,4-thiadiazol-3-yl)-4-(pyrimidin-4-ylamino)benzamide

Utilizing the same sequence of reactions as described in Example 17 and starting with 3-fluoro-4-(trifluoromethoxy)phenylboronic acid and using Pd₂(dba)₃/2-dicyclohexylphosphino)biphenyl as the catalyst in Step A, the title compound was prepared. LC-MS m/z (M+H)⁺ 477, (M−H)⁻ 475.

### Example 87

### N-(3-(2-Fluoro-5-(trifluoromethoxy)phenyl)-1,2,4-thiadiazol-5-yl)-4-(pyrimidin-4-ylamino)benzamide

Utilizing the same sequence of reactions as described in Example 12 and starting with 2-fluoro-5-(trifluoromethoxy)phenylboronic acid, the title compound was prepared. LC-MS m/z (M+H)⁺ 477, (M−H)⁻ 475.

### Example 88

### 4-(6-(Dimethylamino)pyrimidin-4-ylamino)-N-(3-(2-fluoro-5-(trifluoromethoxy)phenyl)-1,2,4-thiadiazol-5-yl)benzamide

Utilizing the same sequence of reactions as described in Example 12 and starting with 2-fluoro-5-(trifluoromethoxy)phenylboronic acid in Step A and methyl 4-(6-(dimethylamino)pyrimidin-4-ylamino)benzoate (from Preparation C) in Step D, the title compound was prepared. LC-MS m/z (M+H)⁺ 520, (M−H)⁻ 518.

### Example 89

### N-(4-(2-Fluoro-3-methoxyphenyl)thiazol-2-yl)-4-(pyrimidin-4-ylamino)benzamide

Utilizing the same sequence of reactions as described in Example 51 and starting with 2-fluoro-3-(methoxy)phenylboronic acid, the title compound was prepared. LC-MS m/z (M+H)⁺ 422, (M−H)⁻ 420.

### Example 90

### 4-(6-(N-Methoxy-N-methylamino)pyrimidin-4-ylamino)-N-(3-(4-fluoro-3-(trifluoromethyl)phenyl)-1,2,4-thiadiazol-5-yl)benzamide

Utilizing the same sequence of reactions as described in Preparation B and Example 10 and starting with N-methoxymethanamine, the title compound was prepared. LC-MS m/z (M+H)⁺ 520, (M−H)⁻ 518.

### Example 91

### 4-(6-(Methylamino)pyrimidin-4-ylamino)-N-(3-(4-fluoro-3-(trifluoromethyl)phenyl)-1,2,4-thiadiazol-5-yl)benzamide

The title compound was isolated as a side product from the last step of Example 90. LC-MS m/z (M+H)⁺ 490, (M−H)⁻ 488.

### Example 92

### N-(3-Phenylisothiazol-5-yl)-4-(pyrimidin-4-ylamino)benzamide

Utilizing the same procedure as described in Example 10 and starting with the hydrochloride salt of 3-phenylisothiazol-5-amine (prepared from the parent amine (from M. Beringer et al., Helv. Chim. Acta 1966, 49, 2466) and HCl gas in CH₂Cl₂), the title compound was prepared. LC-MS m/z (M+H)⁺ 374, (M−H)⁻ 372.

### Example 93

### N-(3-(4-Chlorophenyl)isothiazol-5-yl)-4-(pyrimidin-4-ylamino)benzamide

Utilizing the same procedure as described in Example 10 and starting with the hydrochloride salt of 3-(4-chlorophenyl)isothiazol-5-amine (prepared from the parent amine (from the method of M. Beringer et al., Helv. Chim. Acta 1966, 49, 2466) and HCl gas in CH₂Cl₂), the title compound was prepared. LC-MS m/z (M+H)⁺ 408, (M−H)⁻ 406.

### Example 94

### N-(3-(3,4,5-Trifluorophenyl)-1,2,4-thiadiazol-5-yl)-4-(pyrimidin-4-ylamino)benzamide

### Step A: 2-((5-Formamido)-1,2,4-thiadiazol-3-yl)sulfanyl)-N,N-dimethylacetamide

Utilizing the same procedure as described in Preparation G and starting with 2-(5-amino-1,2,4-thiadiazol-3-ylthio)-N,N-dimethylacetamide (from Preparation D, Step 2), the title compound was prepared. LC-MS m/z (M+H)⁺ 271.

### Step B: N′-(3-(3,4,5-Trifluorophenyl)-1,2,4-thiadiazol-5-yl)-N N-dimethylformamidine

Utilizing the same procedure as described in Example 12, Step A and starting with 3,4,5-trifluorophenylboronic acid, the title compound was prepared. LC-MS m/z (M+H)⁺ 287.

### Step C: 3-(3,4,5-Trifluorophenyl)-1,2,4-thiadiazol-5-amine

Utilizing the same procedure as described in Example 17, Step B, the title compound was prepared. LC-MS m/z (M+H)⁺ 232, (M−H)⁻ 230.

### Step D: N-(3-(3,4,5-Trifluorophenyl)-1,24-thiadiazol-5-yl)-4-(pyrimidin-4-ylamino)benzamide

Utilizing the same procedure as described in Example 10 and starting with the hydrochloride salt of 3-(3,4,5-trifluorophenyl)-1,2,4-thiadiazol-5-amine (prepared from the parent amine (from the method of M. Beringer et al., Helv. Chim. Acta 1966, 49, 2466) and HCl gas in CH₂Cl₂), the title compound was prepared. LC-MS m/z (M+H)⁺ 429, (M−H)⁻ 427.

### Example 95

### N-(3-(3-Chloro-4-fluorophenyl)-1,2,4-thiadiazol-5-yl)-4-(pyrimidin-4-ylamino)benzamide

Utilizing the same sequence of reactions as described in Example 94 and starting with 3-chloro-4-fluorophenylboronic acid in Step B, the title compound was prepared. LC-MS m/z (M+H)⁺ 427, (M−H)⁻ 425.

### Example 96

### N-(3-(2-Fluoro-5-(trifluoromethyl)phenyl)-1,2,4-thiadiazol-5-yl)-4-(pyrimidin-4-ylamino)benzamide

Utilizing the same sequence of reactions as described in Example 94 and starting with 2-fluoro-5-(trifluoromethyl)phenylboronic acid in Step B, the title compound was prepared. LC-MS m/z (M+H)⁺ 461, (M−H)⁻ 459.

### Example 97

### N-(3-(2-Fluoro-3-(trifluoromethoxy)phenyl)-1,2,4-thiadiazol-5-yl)-4-(pyrimidin-4-ylamino)benzamide

Utilizing the same sequence of reactions as described in Example 94 and starting with 2-fluoro-3-(trifluoromethoxy)phenylboronic acid in Step B, the title compound was prepared. LC-MS m/z (M+H)⁺ 477.

### Example 98

### 4-(6-((S)-2-Hydroxy-2-((S)-2,2-dimethyl-1,3-dioxolan-4-yl)ethylamino)pyrimidin-4-ylamino)-N-(3-(4-fluoro-3-(trifluoromethyl)phenyl)-1,2,4-thiadiazol-5-yl)benzamide

4-(6-Chloropyrimidin-4-ylamino)-N-(3-(4-fluoro-3-(trifluoromethyl)phenyl)-1,2,4-thiadiazol-5-yl)benzamide (396 mg, 0.80 mmol, from Example 82) and (S)-2-amino-1-((S)-2,2-dimethyl-1,3-dioxolan-4-yl)ethanol (795 mg, 5.0 mmol, from L. V. Nechev et al., Chem. Res. Toxicol. 2001, 14, 279) were heated together in dioxane until LC-MS showed the reaction to be complete (4 days). The cooled mixture was concentrated, the residue chromatographed, and purified material triturated with water. Filtration and drying gave the title compound 196 mg (40%). LC-MS m/z (M+H)⁺ 620, (M−H)⁻ 618.

### Example 99

### 4-(6-((2S,3S)-2,3,4-Trihydroxybutylamino)pyrimidin-4-ylamino)-N-(3-(4-fluoro-3-(trifluoromethyl)phenyl)-1,2,4-thiadiazol-5-yl)benzamide

4-(6-((S)-2-Hydroxy-2-((S)-2,2-dimethyl-1,3-dioxolan-4-yl)ethylamino)pyrimidin-4-ylamino)-N-(3-(4-fluoro-3-(trifluoromethyl)phenyl)-1,2,4-thiadiazol-5-yl)benzamide (165 mg, 0.27 mmol, from Example 98) was treated with 1N HCl (1 mL) in methanol (10 mL) for 18 h. The mixture was concentrated to a brown solid. This was suspended in THF and treated with MP-Carbonate® for 18 h. The resin was filtered off and the filtrate concentrated to a brown solid that was triturated with ether, filtered and dried to give the title compound 45 mg (30%). LC-MS m/z (M+H)⁺ 580.

### Examples 100-110

Utilizing the same procedure as described in Example 98 and starting with the appropriate amine, the following examples were prepared:

### Example 100

### 4-(6-(N-(2-(Dimethylamino)ethyl)-N-methylamino)pyrimidin-4-ylamino)-N-(3-(4-fluoro-3-(trifluoromethyl)phenyl)-1,2,4-thiadiazol-5-yl)benzamide

LC-MS m/z (M+H)⁺ 561, (M−H)⁻ 559.

### Example 101

### 4-(6-(N-(2-(Hydroxy)ethyl)-N-methylamino)pyrimidin-4-ylamino)-N-(3-(4-fluoro-3-(trifluoromethyl)phenyl)-1,2,4-thiadiazol-5-yl)benzamide

LC-MS m/z (M+H)⁺ 534, (M−H)⁻ 532.

### Example 102

### 4-(6-(N-(2,3-(Dihydroxy)propyl)-N-methylamino)pyrimidin-4-ylamino)-N-(3-(4-fluoro-3-(trifluoromethyl)phenyl)-1,2,4-thiadiazol-5-yl)benzamide

LC-MS m/z (M+H)⁺ 564, (M−H)⁻ 562.

### Example 103

### 4-(6-(N-(2,3-(Dihydroxy)propyl)-amino)pyrimidin-4-ylamino)-N-(3-(4-fluoro-3-(trifluoromethyl)phenyl)-1,2,4-thiadiazol-5-yl)benzamide

LC-MS m/z (M+H)⁺ 550, (M−H)⁻ 548.

### Example 104

### 4-{6-[Bis-(2-hydroxy-ethyl)-amino]-pyrimidin-4-ylamino}-N-[3-(4-fluoro-3-trifluoromethyl-phenyl)-[1,2,4]thiadiazol-5-yl]-benzamide

LC-MS m/z (M+H)⁺ 564, (M−H)⁻ 562.

### Example 105

### 4-(6-(1,3-Dihydroxypropan-2-ylamino)pyrimidin-4-ylamino)-N-(3-(4-fluoro-3-(trifluoromethyl)phenyl)-1,2,4-thiadiazol-5-yl)benzamide

LC-MS m/z (M+H)⁺ 550, (M−H)⁻ 548.

### Example 106

### 4-(6-((R)-1-Hydroxypropan-2-ylamino)pyrimidin-4-ylamino)-N-(3-(4-fluoro-3-(trifluoromethyl)phenyl)-1,2,4-thiadiazol-5-yl)benzamide

LC-MS m/z (M+H)⁺ 534, (M−H)⁻ 532.

### Example 107

### 4-(6-((S)-1-Hydroxypropan-2-ylamino)pyrimidin-4-ylamino)-N-(3-(4-fluoro-3-(trifluoromethyl)phenyl)-1,2,4-thiadiazol-5-yl)benzamide

LC-MS m/z (M+H)⁺ 534, (M−H)⁻ 532.

### Example 108

### 4-{6-[Bis-(3-hydroxy-propyl)-amino]-pyrimidin-4-ylamino}-N-[3-(4-fluoro-3-trifluoromethyl-phenyl)-[1,2,4]thiadiazol-5-yl]-benzamide

LC-MS m/z (M+H)⁺ 592, (M−H)⁻ 590.

### Example 109

### 4-(6-(3-Hydroxypropylamino)pyrimidin-4-ylamino)-N-(3-(4-fluoro-3-(trifluoromethyl)phenyl)-1,2,4-thiadiazol-5-yl)benzamide

LC-MS m/z (M+H)⁺ 534, (M−H)⁻ 532.

### Example 110

### 4-(6-(2-Hydroxyethylamino)pyrimidin-4-ylamino)-N-(3-(4-fluoro-3-(trifluoromethyl)phenyl)-1,2,4-thiadiazol-5-yl)benzamide

LC-MS m/z (M+H)⁺ 520.

### Example 111

### 4-(6-(2,3-Dihydroxypropylthio)pyrimidin-4-ylamino)-N-(3-(4-fluoro-3-(trifluoromethyl)phenyl)-1,2,4-thiadiazol-5-yl)benzamide

Using the same procedure as described in Example 98 but starting with 3-mercaptopropane-1,2-diol instead of an amine and cesium carbonate (2 equivalents), the title compound was prepared. LC-MS m/z (M+H)⁺ 567, (M−H)⁻ 565.

### Examples 112 and 113

### 4-{6-[N-(2R and 2S)-(2,3-Dihydroxy-propyl)-N-methyl-amino]-pyrimidin-4-ylamino}-N-[3-(4-fluoro-3-trifluoromethyl-phenyl)-[1,2,4]thiadiazol-5-yl]-benzamide

4-{6-[N-(2,3-Dihydroxy-propyl)-N-methyl-amino]-pyrimidin-4-ylamino}-N-[3-(4-fluoro-3-trifluoromethyl-phenyl)-[1,2,4]thiadiazol-5-yl]-benzamide (from Example 102) was chromatographed on Chiralpak AD® to give the pure enantiomers (absolute stereochemistry not assigned)

### Isomer 1: LC-MS m/z (M+H)⁺ 564, (M−H)⁻ 562.

### Isomer 2: LC-MS m/z (M+H)⁺ 564, (M−H)⁻ 562.

### Example 114

### 4-(2-Methylpyrimidin-4-ylamino)-N-(4-(2-fluoro-3-(trifluoromethyl)phenyl)thiazol-2-yl)benzamide

Utilizing the same sequence of reactions as described in Example 60 and starting with 2-methylpyrimidin-4-amine, the title compound was prepared. LC-MS m/z (M+H)⁺ 474, (M−H)⁻ 472.

### Example 115

### N-(3-(3,5-Bis(trifluoromethyl)phenyl)-1,2,4-thiadiazol-5-yl)-4-(pyrimidin-4-ylamino)benzamide

Utilizing the same procedure as described in Example 3, Step F and starting with 3-(3,5-bis(trifluoromethyl)phenyl)-1,2,4-thiadiazol-5-amine (commercial), the title compound was prepared. LC-MS m/z (M+H)⁺ 511, (M−H)⁻ 509.

### Example 116

### N-(5-(2-Fluoro-3-(trifluoromethyl)phenyl)thiazol-2-yl)-4-(pyrimidin-4-ylamino)benzamide

### Step A: tert-Butyl 4-methoxybenzyl5-bromothiazol-2-ylcarbamate

Utilizing the same procedure described in Preparation H, Step 3 and starting with tert-butyl 5-bromothiazol-2-ylcarbamate (from G. H. Kuo et al., WO 2002/024681), the title compound was prepared. LC-MS m/z (M+H)⁺ 399/401.

### Step B: tert-butyl 4-methoxybenzyl5-(2-fluoro-3-(trifluoromethyl)phenyl)thiazol-2-ylcarbamate

Utilizing the same procedure as described in Example 51, Step A and starting with tert-butyl 4-methoxybenzyl5-bromothiazol-2-ylcarbamate and 2-(2-fluoro-3-(trifluoromethyl)phenyl)-4,4,5,5-tetramethyl-1,3,2-dioxaborolane, the title compound was prepared. LC-MS m/z (M+H)⁺ 483.

### Step C: 5-(2-fluoro-3-(trifluoromethyl)phenyl)thiazol-2-amine

Utilizing the same procedure as described in Example 51, Step B, the title compound was prepared. LC-MS m/z (M+H)⁺ 263, (M−H)⁻ 261.

### Step D: N-(5-(2-Fluoro-3-(trifluoromethyl)phenyl)thiazol-2-yl)-4-(pyrimidin-4-ylamino)benzamide

Utilizing the same procedure as described in Example 3, Step F, the title compound was prepared. LC-MS m/z (M+H)⁺ 460, (M−H)⁻ 458.

### Example 117

### N-(5-(4-Fluoro-3-(trifluoromethyl)phenyl)thiazol-2-yl)-4-(pyrimidin-4-ylamino)benzamide

### Step A: 5-(4-Fluoro-3-(trifluoromethyl)phenyl)thiazol-2-amine

Utilizing the same sequence of reactions as described in Example 116, Steps B and C and starting with 2-(4-fluoro-3-(trifluoromethyl)phenyl)-4,4,5,5-tetramethyl-1,3,2-dioxaborolane in Step B, the title compound was prepared. LC-MS m/z (M+H)⁺ 263, (M−H)⁻ 261.

### Step B: N-(5-(4-Fluoro-3-(trifluoromethyl)phenyl)thiazol-2-yl)-4-Iodobenzamide

Utilizing the same procedure as described in Example 15, Step C and starting with 5-(4-Fluoro-3-(trifluoromethyl)phenyl)thiazol-2-amine, the title compound was prepared. LC-MS m/z (M−H)⁻ 491.

### Step C: N-(5-(4-Fluoro-3-(trifluoromethyl)phenyl)thiazol-2-yl)-4-(pyrimidin-4-ylamino)benzamide

Utilizing the same procedure as described in Example 1, Step F and starting with N-(5-(4-fluoro-3-(trifluoromethyl)phenyl)thiazol-2-yl)-4-iodobenzamide, the title compound was prepared. LC-MS m/z (M+H)⁺ 460, (M−H)⁻ 458.

### Example 118

### 4-(1,3,4-Thiadiazol-2-ylamino)-N-(4-(2-fluoro-3-(trifluoromethyl)phenyl)thiazol-2-yl)benzamide

### Step A: N-(4-(2-Fluoro-3-(trifluoromethyl)phenyl)thiazol-2-yl)-4-iodobenzamide

Utilizing the same procedure as described in Example 15, Step C and starting with 4-(2-fluoro-3-(trifluoromethyl)phenyl)thiazol-2-amine (from Example 18, Step B), the title compound was prepared. LC-MS m/z (M+H)⁺ 493, (M−H)⁻ 491.

### Step B: 4-(1,3,4-Thiadiazol-2-ylamino)-N-(4-(2-fluoro-3-(trifluoromethyl)phenyl)thiazol-2-yl)benzamide

Utilizing the same procedure as described in Example 1, Step F and starting with N-(4-(2-fluoro-3-(trifluoromethyl)phenyl)thiazol-2-yl)-4-iodobenzamide and 1,3,4-thiadiazol-2-amine, the title compound was prepared. LC-MS m/z (M+H)⁺ 466, (M−H)⁻ 464.

### Example 119

### N-[1-(4-Fluoro-3-trifluoromethyl-phenyl)-1H-pyrazol-3-yl]-4-(pyrimidin-4-ylamino)-benzamide

### Step A: 4-fluoro-3-trifluoromethyl phenyl hydrazine

A suspension of 4-fluoro-3-trifluoromethyl aniline (13.9 g, 0.75 mol) in 1:1 glacial acetic acid/conc. HCl was cooled to 0° C. A solution of sodium nitrite (5.7 g, 0.083 mol) in water (15 mL) was added dropwise over 30 min while maintaining the temperature at 0° C. After stirring for an additional 30 min at 0° C., a solution of stannous chloride dihydrate (52.0 g, 0.225 mol) in conc. HCl (100 mL) was added. The resulting solution was stirred for 30 minutes and filtered. The filtrate was basified to pH 12 and extracted with ether. The ether layer was washed with water, brine, dried and concentrated in vacuo to yield 11.0 g of the title compound. LC-MS m/z (M+H)⁺ 195.

### Step B: 1-(4-Fluoro-3-trifluoromethyl-phenyl)-1H-pyrazol-3-ylamine

To a mixture of potassium tert-butoxide (2.8 g, 0.025 mol) in tert-butanol (20 mL) was added 4-fluoro-3-trifluoromethyl phenyl hydrazine (1.04 g, 0.01 mol). After stirring for 5 minutes a solution of 2,3-dibromopropionitrile (2.12 g, 0.01 mol) in tert-butanol (10 mL) was added and the resulting mixture was refluxed for 3 h under an atmosphere of nitrogen. Water (20 mL) was added and the mixture evaporated to dryness. The residue was extracted with ethyl acetate and the organic layer washed with water, brine and dried. Purification on silica gel using ethyl acetate/hexanes yielded 0.4 g of title compound. LC-MS m/z (M+H)⁺ 246.

### Step C: N-[1-(4-Fluoro-3-trifluoromethyl-Phenyl)-1H-pyrazol-3-yl]-4-(pyrimidin-4-ylamino)-benzamide

Utilizing the same procedure as described in Example 3, Step F and starting with 1-(4-fluoro-3-trifluoromethyl-phenyl)-1H-pyrazol-3-ylamine, the title compound was prepared. LC-MS m/z (M+H)⁺ 443.

### Example 120

### N-[1-(2-Fluoro-3-trifluoromethyl-phenyl)-1H-pyrazol-3-yl]-4-(pyrimidin-4-ylamino)-benzamide

Using the same sequence of reactions as described in Example 119 and starting with 2-fluoro-3-trifluoromethyl aniline (in Step A), the title compound was prepared. LC-MS m/z (M+H)⁺ 443.

### Example 121

### 4-(6-Cyclopropylmethoxy-pyrimidin-4-ylamino)-N-[3-(4-fluoro-3-trifluoromethyl-phenyl)-[1,2,4]thiadiazol-5-yl]-benzamide

### Step A: 4-(6-Cyclopropoxy-pyrimidin-4-ylamino)-benzoic acid

Cyclopropyl-methanol (260 mg, 3.6 mmol) was added to 4-(6-Chloropyrimidin-4-ylamino)-benzoic acid (see preparation A, step 1) (150 mg, 0.6 mmol) and NaH (60% in mineral oil; 144 mg, 3.6 mmol) in 1,4 dioxane (2 mL) at room temperature. The reaction was warmed to 80° C. for 2 h and placed in a microwave reactor at 180° C. for 1 h. The cooled reaction mixture is diluted with water and washed with ether. The aqueous was and acidified with 1N HCL to pH ˜1 and extracted with EtOAc, dried over MgSO₄, filtered and concentrated to a white solid that was used directly.

Note: a) if a solid formed upon adjusting the pH to 1 it was collected and used without further purification; b) the reaction can be carried out in DMF instead of 1,4-dioxane; c) and in some cases the reaction was not run in the microwave.

### Step B: 4-(6-Cyclopropoxy-pyrimidin-4-ylamino)-benzoic acid methyl ester

A solution of the above 4-(6-Cyclopropoxy-pyrimidin-4-ylamino)-benzoic acid (0.6 mmol) and thionyl chloride (88 mg, 0.72 mmol) in 1,4-dioxane (3 mL) was stirred at 80° C. for 4 hours. Methanol (5 mL) was added and the reaction stirred for an additional 2 h at room temperature. Water was added and the mixture was extracted with EtOAc, dried over MgSO₄, concentrated and chromatographed to yield 100 mg (56% for two steps) of the title compound as an off-white solid.

### Step C: 4-(6-Cyclopropylmethoxy-pyrimidin-4-ylamino)-N-[3-(4-fluoro-3-trifluoromethyl-phenyl)-[1,2,4]thiadiazol-5-yl]-benzamide

3-(4-Fluoro-3-trifluoromethyl-phenyl)-[1,2,4]thiadiazol-5-yl-aminomethyl aluminum chloride (0.9 mL of a 0.272M solution in toluene; 0.24 mmol) prepared from 3-(4-Fluoro-3-trifluoromethyl-phenyl)-[1,2,4]thiadiazol-5-yl-ammonium chloride by the method of Levin et al (Syn. Comm. 1982, 12, 989) was added to a solution of 4-(6-Cyclopropoxy-pyrimidin-4-ylamino)-benzoic acid methyl ester (60 mg, 0.2 mmol) in toluene (2 mL) and heated to 90° C. for 12-18 h. The reaction was cooled to room temperature and then quenched by addition of water followed by 1N HCl to adjust the pH to −1. This mixture was stirred for 0.5 h and then extracted with EtOAc. The combined organics were dried over MgSO₄ and concentrated. Chromatography yielded 45 mg (43%) of the title compound as a white solid. LC-MS m/z (M+H)⁺ 531.3, (M−H)⁻ 529.2.

### Examples 122-132

Utilizing the same sequence of reactions as described in Example 121 and starting with the corresponding alkoxy-pyrimidin-4-ylamine prepared as in preparation J, the following examples were prepared:

### Example 122

### 4-[6-(2-Benzyloxy-ethoxy)-pyrimidin-4-ylamino]-N-[3-(4-fluoro-3-trifluoromethyl-phenyl)-[1,2,4]thiadiazol-5-yl]-benzamide

LC-MS m/z (M+H)⁺ 611.3.

### Example 123

### N-[3-(4-Fluoro-3-trifluoromethyl-phenyl)-[1,2,4]thiadiazol-5-yl]-4-(6-methoxy-pyrimidin-4-ylamino)-benzamide

LC-MS m/z (M+H)⁺ 491.3, (M−H)⁻ 489.3.

### Example 124

### N-[3-(4-Fluoro-3-trifluoromethyl-phenyl)-[1,2,4]thiadiazol-5-yl]-4-(6-isopropoxy-pyrimidin-4-ylamino)-benzamide

LC-MS m/z (M+H)⁺ 519.3, (M−H)⁻ 517.3.

### Example 125

### 4-(6-Ethoxy-pyrimidin-4-ylamino)-N-[3-(4-fluoro-3-trifluoromethyl-phenyl)-[1,2,4]thiadiazol-5-yl]-benzamide

LC-MS m/z (M−H)⁻ 503.2.

### Example 126

### N-[3-(4-Fluoro-3-trifluoromethyl-phenyl)-[1,2,4]thiadiazol-5-yl]-4-[6-(2-methoxy-ethoxy)-pyrimidin-4-ylamino]-benzamide

LC-MS m/z (M+H)⁺ 549.3, (M−H)⁻ 547.3.

### Example 127

### N-[3-(4-Fluoro-3-trifluoromethyl-phenyl)-[1,2,4]thiadiazol-5-yl]-4-[6-(2-methoxy-1-methyl-ethoxy)-pyrimidin-4-ylamino]-benzamide

LC-MS m/z (M+H)⁺ 535.3, (M−H)⁻ 533.3.

### Example 128

### 4-(6-Cyclobutylmethoxy-pyrimidin-4-ylamino)-N-[3-(4-fluoro-3-trifluoromethyl-phenyl)-[1,2,4]thiadiazol-5-yl]-benzamide

LC-MS m/z (M+H)⁺ 545.3, (M−H)⁻ 543.3.

### Example 129

### N-[3-(4-Fluoro-3-trifluoromethyl-phenyl)-[1,2,4]thiadiazol-5-yl]-4-[2-(2-methoxy-1-methyl-ethoxy)-Pyridin-4-ylamino]-benzamide

LC-MS m/z (M+H)⁺ 548.4, (M−H)⁻ 546.3.

### Example 130

### 4-[6-(Azetidin-3-yloxy)-pyrimidin-4-ylamino]-N-[3-(4-fluoro-3-trifluoromethyl-phenyl)-[1,2,4]thiadiazol-5-yl]-benzamide

LC-MS m/z (M+H)⁺ 532.3, (M−H)⁻ 530.3.

### Example 131

### 4-(6-Cyclohexylmethoxy-pyrimidin-4-ylamino)-N-[3-(4-fluoro-3-trifluoromethyl-phenyl)-[1,2,4]thiadiazol-5-yl]-benzamide

LC-MS m/z (M+H)⁺ 573.3.

### Example 132

### N-[3-(4-Fluoro-3-trifluoromethyl-phenyl)-[1,2,4]thiadiazol-5-yl]-4-(2-methoxy-pyridin-4-ylamino)-benzamide

LC-MS m/z (M+H)⁺ 490.3, (M−H)⁻ 488.2.

### Example 133

### N-[3-(4-Fluoro-3-trifluoromethyl-phenyl)-[1,2,4]thiadiazol-5-yl]-4-[6-(2-hydroxy-ethoxy)-pyrimidin-4-ylamino]-benzamide

A Parr flask containing 4-[6-(2-Benzyloxy-ethoxy)-pyrimidin-4-ylamino]-N-[3-(4-fluoro-3-trifluoromethyl-phenyl)-[1,2,4]thiadiazol-5-yl]-benzamide (500 mg, 0.82 mmol), (prepared as in Example 121) and 10% palladium(II) hydroxide on carbon (460 mg) in methanol (25 mL) was charged with hydrogen (45 psi) and warmed to 40° C. for 24 h. The catalyst was removed by filtration, the mixture concentrated and chromatographed to yield 40 mg (9.5%) of the title compound as a white solid. LC-MS m/z (M+H)⁺ 521.3.

### Example 134

### N-[3-(4-Fluoro-3-trifluoromethyl-phenyl)-[1,2,4]thiadiazol-5-yl]-4-(1H-[2,3]triazolo[4,5-d]pyrimidin-7-ylamino)-benzamide

### Step A: 4-(1H-[1,2,3]-Triazolo[4,5-d]pyrimidin-7-ylamino)-benzoic acid methyl ester

Using the same procedure as Example 1 step F, 4-iodo-benzoic acid methyl ester (200 mg) was converted to 50 mg of the titled compound as a yellow solid

### Step B: N-[3-(4-Fluoro-3-trifluoromethyl-phenyl)-[1,2,4]-thiadiazol-5-yl]-4-(1H-[1,2,3]triazolo[4,5-d]pyrimidin-7-ylamino)-benzamide

Using the same procedure as Example 121 step C, 50 mg of 4-(1H-[1,2,3]triazolo[4,5-d]pyrimidin-7-ylamino)-benzoic acid methyl ester was converted to 15 mg of the titled compound as an off-white solid.

LC-MS m/z (M+H)⁺ 501.3.

### Examples 135 and 136

Utilizing the same sequence of reactions as described in Example 14 and starting with the corresponding heterocycle, the following examples were prepared:

### Example 135

### N-[3-(4-Fluoro-3-trifluoromethyl-phenyl)-[1,2,4]-thiadiazol-5-yl]-4-(9H-purin-6-ylamino)-benzamide

LC-MS m/z (M+H)⁺ 501.2.

### Example 136

### N-[3-(4-Fluoro-3-trifluoromethyl-phenyl)-[1,2,4]thiadiazol-5-yl]-4-(1H-[1,2,3]triazolo[4,5-d]pyrimidin-7-ylamino)-benzamide

LC-MS m/z (M+H)⁺ 501.8, (M−H)⁻ 500.9.

### Example 137

### 4-(2-Chloro-pyridin-4-ylamino)-N-[3-(4-fluoro-3-trifluoromethyl-phenyl)-[1,2,4]-thiadiazol-5-yl]-benzamide

Using the procedures described for the preparation of Example 82 starting with 2-chloro-pyridin-yl-amine the titled compound was prepared. LC-MS m/z (M+H)⁺ 460, (M−H)⁻ 458.

### Examples 138-144

Using the procedures described for the preparation of Example 82 starting with the appropriate thiadiazole or methyl thiazole paired with the appropriate chloro-pyrimidine or chloro-pyridine, the following compounds were prepared.

### Example 138

### 4-(6-Chloro-pyrimidin-4-yamino)-N-[4-(4-fluoro-3-trifluoromethyl-phenyl)-5-methyl-thiazol-2-yl]-benzamide

LC-MS m/z (M+H)⁺ 494.3.

### Example 139

### 4 N-[3-(3-Bromo-4-fluoro-phenyl)-[1,2,4]-thiadiazol-5-yl]-4-(6-chloro-pyrimidin-4-ylamino)-benzamide

MS m/z (M−H)⁻ 506.8.

### Example 140

### 4-(6-Chloro-pyrimidin-4-ylamino)-N-[5-methyl-4-(3-trifluoromethyl-phenyl)-thiazol-2-yl]-benzamide

LC-MS m/z (M+H)⁺ 460, (M−H)⁻ 458.

### Example 141

### 4-(6-Chloro-pyrimidin-4-ylamino)-N-[4-(2-fluoro-3-trifluoromethyl-phenyl)-5-methyl-thiazol-2-yl]-benzamide

LC-MS m/z (M+H)⁺ 503.3.

### Example 142

### 4-(6-Chloro-pyrimidin-4-ylamino)-N-[4-(2-fluoro-5-trifluoromethyl-phenyl)-5-methyl-thiazol-2-yl]-benzamide

LC-MS m/z (M+H)⁺ 508.2, (M−H)⁻ 501.2.

### Example 143

### N-[3-(3-Butyl-4-fluoro-phenyl)-[1,2,4]thiadiazol-5-yl]-4-(6-chloro-pyrimidin-4-ylamino)-benzamide

LC-MS m/z (M+H)⁺ 483.3, (M−H)⁻ 481.2.

### Example 144

### 4-(6-isopropoxy-pyrimidin-4-ylamino)-N-[3-(4-isopropoxy-3-trifluoromethyl-phenyl)-[1,2,4]thiadiazol-5-yl]-benzamide

A solution of 4-(6-Chloro-pyrimidin-4-ylamino)-N-[3-(4-fluoro-3-trifluoromethyl-phenyl)-[1,2,4]thiadiazol-5-yl]-benzamide (35 mg, 0.07 mmol) (see Example 82) and sodium isopropoxide (0.5 mL of a 0.5M solution in ethanol) was stirred at 90° C. for 12-18 h, another portion of sodium isopropoxide 0.5 1 mL) was added and the reaction was heated in the microwave at 140° C. for an additional 30 min. Reaction was diluted with water and then extracted with EtOAc, dried over MgSO₄ and concentrated. Chromatography yielded 10 mg (26%) of the titled compound as a white solid. LC-MS m/z (M+H)⁺ 559.4, (M−H)⁻ 557.3.

### Examples 145 and 146

Using the same sequence of reactions as described in Example 21, starting with 4-(6-Chloro-pyrimidin-4-ylamino)-N-[3-(4-fluoro-3-trifluoromethyl-phenyl)-[1,2,4]thiadiazol-5-yl]-benzamide (Example 82) and the addition of sodium to the appropriate alcohol, the following examples were prepared.

### Example 145

### 4-(6-Methoxy-pyrimidin-4-ylamino)-N-[3-(4-ethoxy-3-trifluoromethyl-phenyl)-[1,2,4]thiadiazol-5-yl]-benzamide

LC-MS m/z (M+H)⁺ 460, (M−H)⁻ 458.

### Example 146

### 4-(6-Methoxy-pyrimidin-4-ylamino)-N-[3-(4-methoxy-3-trifluoromethyl-phenyl)-[1,2,4]thiadiazol-5-yl]-benzamide

LC-MS m/z (M+H)⁺ 460, (M−H)⁻ 458.

### Example 147

### N-[3-(4-Fluoro-3-trifluoromethyl-phenyl)-[1,2,4]thiadiazol-5-yl]-4-(6-pyrrolidin-1-yl-pyrimidin-4-ylamino)-benzamide

A solution of 4-(6-Chloro-pyrimidin-4-ylamino)-N-[3-(4-fluoro-3-trifluoromethyl-phenyl)-[1,2,4]thiadiazol-5-yl]-benzamide (60 mg, 0.12 mmol) (Example 82) and pyrrolidine (38 mg, 0.54 mmol) in THF were heated in a microwave at 179° C. for 2.5 h. The crude reaction was concentrated and chromatographed to provide the titled compound. LC-MS m/z (M+H)⁺ 529.3, (M−H)⁻ 527.2.

### Example 148

### N-[3-(4-Fluoro-3-trifluoromethyl-phenyl)-[1,2,4]thiadiazol-5-yl]-4-(6-methoxy-pyridin-3-ylamino)-benzamide

Using the procedure from Example 1 step F, N-[3-(4-Fluoro-3-trifluoromethyl-phenyl)-[1,2,4]thiadiazol-5-yl]-4-iodo-benzamide was converted to the title compound in 64% yield. LC-MS m/z (M+H)⁺ 490.3, (M−H)⁻ 488.3.

### Examples 149-152

Using the same sequence of reactions as described in Example 25, starting with the appropriately substituted 4-amino pyridine the following examples were prepared.

### Example 149

### N-[3-(4-Fluoro-3-trifluoromethyl-phenyl)-[1,2,4]thiadiazol-5-yl]-4-(pyridin-4-ylamino)-benzamide

LC-MS m/z (M+H)⁺ 460.3, (M−H)⁻ 458.2.

### Example 150

### 4-(2-Ethoxy-pyridin-4-ylamino)-N-[3-(4-fluoro-3-trifluoromethyl-phenyl)-[1,2,4]-thiadiazol-5-yl]-benzamide

LC-MS m/z (M+H)⁺ 504.3.

### Example 151

### 4-(2-Cyclopropylmethoxy-Pyridin-4-ylamino)-N-[3-(4-fluoro-3-trifluoromethyl-phenyl)-[1,2,4]thiadiazol-5-yl]-benzamide

C-MS m/z (M+H)⁺ 530.3, (M−H)⁻ 528.3.

### Example 152

### N-[3-(4-Fluoro-3-trifluoromethyl-phenyl)-[1,2,4]thiadiazol-5-yl]-4-(5-trifluoromethyl-pyridin-2-ylamino)-benzamide

C-MS m/z (M+H)⁺ 528.3, (M−H)⁻ 526.3.

### Example 153

### N-[4-(2-Fluoro-3-trifluoromethyl-phenyl)-5-methyl-thiazol-2-yl]-4-{6-[3-(4-methyl-piperazin-1-yl)-propylamino]-pyrimidin-4-ylamino}-benzamide

A solution of 4-(6-Chloro-pyrimidin-4-ylamino)-N-[4-(4-fluoro-3-trifluoromethyl-phenyl)-5-methyl-thiazol-2-yl]-benzamide (100 mg, 0.20 mmol) (Example 21) and 3-(4-Methyl-piperazin-1-yl)-propylamine (93 mg, 0.59 mmol) in THF (2.5 mL) was heated at 140° C. for 2 h in the microwave. The reaction mixture was adsorbed onto silica gel and chromatographed to provide 76 mg (61%) of the titled compound. LC-MS m/z (M+H)⁺ 629.4.

### Examples 154-180

Using the same sequence of reactions as described in Example 31, starting with the appropriate Example 18, Example 19 or Example 21 the following compounds were prepared

### Example 154

### N-[4-(2-Fluoro-3-trifluoromethyl-phenyl)-5-methyl-thiazol-2-yl]-4-{6-[3-(2-oxo-pyrrolidin-1-yl)-propylamino]-pyrimidin-4-ylamino}-benzamide

LC-MS m/z (M+H)⁺ 614.4.

### Example 155

### 4-(6-[(2,3-Dihydroxy-propyl)-methyl-amino]-pyrimidin-4-ylamino)-N-[4-(2-fluoro-3-trifluoromethyl-Phenyl)-5-methyl-thiazol-2-yl]-benzamide

LC-MS m/z (M+H)⁺ 577.4.

### Example 156

### 4-(6-Dimethylamino-pyrimidin-4-ylamino)-N-[4-(2-fluoro-3-trifluoromethyl-Phenyl)-5-methyl-thiazol-2-yl]-benzamide

LC-MS m/z (M+H)⁺ 517.3.

### Example 157

### N-[4-(2-Fluoro-3-trifluoromethyl-phenyl)-5-methyl-thiazol-2-yl]-4-(6-methylamino-pyrimidin-4-ylamino)-benzamide

LC-MS m/z (M+H)⁺ 503.3.

### Example 158

### 4-(6-Dimethylamino-pyrimidin-4-ylamino)-N-[5-methyl-4-(3-trifluoromethyl-phenyl)-thiazol-2-yl]benzamide

C-MS m/z (M+H)⁺ 499.3, (M−H)⁻ 497.3.

### Example 159

### 4-[6-(2,3-Dihydroxy-Propylamino)-pyrimidin-4-ylamino]-N-[4-(2-fluoro-3-trifluoromethyl-phenyl)-5-methyl-thiazol-2-yl]-benzamide

LC-MS m/z (M+H)⁺ 563.2, (M−H)⁻ 561.2.

### Example 160

### 4-[6-(4-Ethyl-piperazin-1-yl)-pyrimidin-4-ylamino]-N-[4-(2-fluoro-3-trifluoromethyl-phenyl)-5-methyl-thiazol-2-yl]-benzamide

LC-MS m/z (M+H)⁺ 586.3, (M−H)⁻ 584.3.

### Example 161

### 4-(6-Methylamino-pyrimidin-4-ylamino)-N-[5-methyl-4-(3-trifluoromethyl-phenyl)-thiazol-2-yl]-benzamide

LC-MS m/z (M+H)⁺ 485.3, (M−H)⁻ 483.3.

### Example 162

### 4-[6-(2,3-Dihydroxy-Propylamino)-pyrimidin-4-ylamino]-N-[5-methyl-4-(3-trifluoromethyl-phenyl)-thiazol-2-yl]-benzamide

LC-MS m/z (M+H)⁺ 545.3, (M−H)⁻ 543.3.

### Example 163

### 4-[6-(2,3-Dihydroxy-propylamino)-pyrimidin-4-ylamino]-N-[5-methyl-4-(3-trifluoromethyl-phenyl)-thiazol-2-yl]-benzamide

LC-MS m/z (M+H)⁺ 559.3, (M−H)⁻ 557.3.

### Example 164

### 4-(6-Dimethylamino-pyrimidin-4-ylamino)-N-[5-methyl-4-(3-trifluoromethyl-phenyl)-thiazol-2-yl]-benzamide

LC-MS m/z (M+H)⁺ 596.4, (M−H)⁻ 594.3.

### Example 165

### 4-(6-Dimethylamino-pyrimidin-4-ylamino)-N-[4-(2-fluoro-5-trifluoromethyl-phenyl)-5-methyl-thiazol-2-yl]-benzamide

LC-MS m/z (M+H)⁺ 517.3, (M−H)⁻ 515.3.

### Example 166

### 4-{6-[(2,3-Dihydroxy-propyl)-methyl-amino]-pyrimidin-4-ylamino}-N-[4-(2-fluoro-5-trifluoromethyl-phenyl)-5-methyl-thiazol-2-yl]-benzamide

LC-MS m/z (M+H)⁺ 577.4, (M−H)⁻ 575.3.

### Example 167

### 4-[6-(2,3-Dihydroxy-propylamino)-pyrimidin-4-ylamino]-N-[4-(4-fluoro-3-trifluoromethyl-phenyl)-5-methyl-thiazol-2-yl]-benzamide

LC-MS m/z (M+H)⁺ 563.3, (M−H)⁻ 561.3.

### Example 168

### N-[4-(4-Fluoro-3-trifluoromethyl-phenyl)-5-methyl-thiazol-2-yl]-4-(6-methylamino-pyrimidin-4-ylamino)-benzamide

LC-MS m/z (M+H)⁺ 503.3, (M−H)⁻ 501.3.

### Example 169

### N-[4-(4-Fluoro-3-trifluoromethyl-phenyl)-5-methyl-thiazol-2-yl]-4-(6-methylamino-pyrimidin-4-ylamino)-benzamide

LC-MS m/z (M+H)⁺ 614.3, (M−H)⁻ 612.3.

### Example 170

### N-[4-(4-Fluoro-3-trifluoromethyl-phenyl)-5-methyl-thiazol-2-yl]-4-(6-methylamino-pyrimidin-4-ylamino)-benzamide

LC-MS m/z (M+H)⁺ 600.5.

### Example 171

### N-[4-(4-Fluoro-3-trifluoromethyl-phenyl)-5-methyl-thiazol-2-yl]-4-{6-[3-(4-methyl-piperazin-1-yl)-Propylamino]-pyrimidin-4-ylamino}-benzamide

LC-MS m/z (M+H)⁺ 629.4, (M−H)⁻ 627.3.

### Example 172

### 4-(6-Dimethylamino-pyrimidin-4-ylamino)-N-[4-(4-fluoro-3-trifluoromethyl-phenyl)-5-methyl-thiazol-2-yl]-benzamide

LC-MS m/z (M+H)⁺ 517.3, (M−H)⁻ 515.3.

### Example 173

### 4-{6-[(2,3-Dihydroxy-propyl)-methyl-amino]-pyrimidin-4-ylamino}-N-[4-(4-fluoro-3-trifluoromethyl-Phenyl)-5-methyl-thiazol-2-yl]-benzamide

LC-MS m/z (M+H)⁺ 577.3, (M−H)⁻ 575.3.

### Example 174

### N-[3-(3-Butyl-4-fluoro-phenyl)-[1,2,4]thiadiazol-5-yl]-4-(6-dimethylamino-pyrimidin-4-ylamino)-benzamide

LC-MS m/z (M+H)⁺ 492.4, (M−H)⁻ 490.3.

### Example 175

### 4-(6-[Bis-(3-hydroxy-propyl)-amino]-pyrimidin-4-ylamino)-N-[3-(3-butyl-4-fluoro-phenyl)-[1,2,4]thiadiazol-5-yl]-benzamide

LC-MS m/z (M+H)⁺ 580.4, (M−H)⁻ 578.3.

### Example 176

### 2-Butyl-4-[4-(2-{4-[3-(1,3-dimethyl-pentyl)-benzyl]-phenyl}-allyl)-cyclopenta-1,4-dienyl]-1-methyl-benzene

LC-MS m/z (M+H)⁺ 552.4, (M−H)⁻ 550.3.

### Example 177

### N-[3-(3-Butyl-4-fluoro-phenyl)-[1,2,4]thiadiazol-5-yl]-4-(6-methylamino-pyrimidin-4-ylamino)-benzamide

LC-MS m/z (M+H)⁺ 478.4, (M−H)⁻ 476.3.

### Example 178

### N-[3-(3-Butyl-4-fluoro-phenyl)-[1,2,4]thiadiazol-5-yl]-4-{6-[3-(2-oxo-pyrrolidin-1-yl)-propylamino]-pyrimidin-4-ylamino}-benzamide

LC-MS m/z (M+H)⁺ 489.4, (M−H)⁻ 487.4.

### Example 179

### N-[5-Methyl-4-(2-fluoro-3-trifluoromethyl-Phenyl)-thiazol-2-yl]-4-(pyrimidin-4-ylamino)-benzamide

4-(Pyrimidin-4-ylamino)-benzoic acid methyl ester (Preparation N, 0.19 g, 0.81 mmol) and 5-methyl-4-(2-fluoro-3-trifluoromethyl-phenyl)-thiazol-2-yl-ammonium chloride (Preparation L: 0.31 g, 1.1 mmol) were converted to the title compound (157 mg, 42%) using the procedure described in Example 121 step C. LC-MS m/z (M+H)⁺ 474.3, (M−H)⁻ 472.3.

### Examples 180-182

Using the same procedure described for Example 179; starting with the appropriate methyl thiazole (Preparation L) the following compounds were prepared.

### Example 180

### 4 N-[4-(4-Fluoro-3-trifluoromethyl-phenyl)-5-methyl-thiazol-2-yl]-4-(pyrimidin-4-ylamino)-benzamide

LC-MS m/z (M+H)⁺ 474.3.

### Example 181

### N-[5-Methyl-4-(3-trifluoromethyl-phenyl)-thiazol-2-yl]-4-(pyrimidin-4-ylamino)-benzamide

LC-MS m/z (M+H)⁺ 456.3, (M−H)⁻ 454.3.

### Example 182

### N-[3-(3-Bromo-4-fluoro-phenyl)-[1,2,4]thiadiazol-5-yl]-4-(pyrimidin-4-ylamino)-benzamide

LC-MS m/z (M+H)⁺ 473.2, (M−H)⁻ 471.1.

### Example 183

### N-[4-(3-ethyl-2-fluoro-phenyl)-thiazol-2-yl]-4-(pyrimidin-4-ylamino)-benzamide

4-(Pyrimidin-4-ylamino)-benzoic acid methyl ester (Preparation N; 120 mg, 0.53 mmol) and 4-(3-Ethyl-2-fluoro-phenyl)-thiazol-2-yl-ammonium chloride (Preparation O: 140 mg, 0.63 mmol) were converted to the title compound (60 mg, 27%) using the procedure described in Example 121 step C. LC-MS m/z (M+H)⁺ 420.3.

### Examples 184-187

Using the same procedure described for Example 183, and starting with the appropriate alkyl thiazoles (Preparation O) the following examples were prepared.

### Example 184

### N-[4-(3-Butyl-2-fluoro-phenyl)-thiazol-2-yl]-4-(pyrimidin-4-ylamino)-benzamide

LC-MS m/z (M+H)⁺ 448.3.

### Example 185

### N-[4-(2-Fluoro-3-isobutyl-phenyl)-thiazol-2-yl]-4-(pyrimidin-4-ylamino)-benzamide

LC-MS m/z (M+H)⁺ 448.4.

### Example 186

### N-[3-(3-Butyl-4-fluoro-phenyl)-[1,2,4]thiadiazol-5-yl]-4-(pyrimidin-4-ylamino)-benzamide

LC-MS m/z (M+H)⁺ 449.3, (M−H)⁻ 447.2.

### Example 187

### N-[4-(2-Fluoro-5-trifluoromethyl-phenyl)-5-methyl-thiazol-2-yl]-4-(pyrimidin-4-ylamino)-benzamide

LC-MS m/z (M+H)⁺ 474.3, (M−H)⁻ 472.2.

### Example 188

### 4-(6-(3-Hydroxyazetidin-1-yl)pyrimidin-4-ylamino)-N-(4-(2-fluoro-3-(trifluoromethoxy)phenyl)thiazol-2-yl)benzamide

Using the same procedure as described in Example 98, 4-(6-chloropyrimidin-4-ylamino)-N-(4-(2-fluoro-3-(trifluoromethoxy)phenyl)thiazol-2-yl)benzamide (prepared by the same sequence of reactions as described in Example 82 and starting with 4-(2-fluoro-3-trifluoromethoxy-phenyl)thiazolyl-2-amine (from Example 54)) and azetidin-3-ol were converted to the title compound. LC-MS m/z (M+H)⁺ 547.

### Example 189

### 4-(6-(3-Hydroxyazetidin-1-yl)pyrimidin-4-ylamino)-N-(3-(4-fluoro-3-(trifluoromethyl)phenyl)-1,2,4-thiadiazol-5-yl)benzamide

Using the same procedure as described in Example 98, 4-(6-chloropyrimidin-4-ylamino)-N-(3-(4-fluoro-3-(trifluoromethyl)phenyl)-1,2,4-thiadiazol-5-yl)benzamide (from Example 82) and azetidin-3-ol were converted to the title compound. LC-MS m/z (M+H)⁺ 532, (M−H)⁻ 530.

### Example 190

### (R)N-[4-(2-fluoro-3-trifluoromethoxy-phenyl)-thiazol-2-yl]-4-(6-[(2,3-dihydroxy-propyl)-methyl-amino]-pyrimidin-4-ylamino)-benzamide

Using the same procedure as described in Example 98, 4-(6-chloropyrimidin-4-ylamino)-N-(4-(2-fluoro-3-(trifluoromethoxy)phenyl)thiazol-2-yl)benzamide (from Example 188) and (R)-3-(methylamino)propane-1,2-diol were converted to the title compound. LC-MS m/z (M+H)⁺ 579, (M−H)⁻ 577.

### Example 191

(S)N-[4-(2-fluoro-3-trifluoromethoxy-phenyl)-4-thiazol-2-yl]-4-(6-[(2,3-dihydroxy-propyl)-methyl-amino]-pyrimidin-4-ylamino)-benzamide

Using the same procedure as described in Example 98, 4-(6-chloropyrimidin-4-ylamino)-N-(4-(2-fluoro-3-(trifluoromethoxy)phenyl)thiazol-2-yl)benzamide (from Example 188) and (S)-3-(methylamino)propane-1,2-diol were converted to the title compound. LC-MS m/z (M+H)⁺ 579.

### Example 192

### N-[4-(2-fluoro-3-trifluoromethyl-phenyl)-thiazol-2-yl]-4-{6-[(2-hydroxy-ethyl)-methyl-amino]-pyrimidin-4-ylamino}-benzamide

Using the same procedure as described in Example 98, 4-(6-chloropyrimidin-4-ylamino)-N-(4-(2-fluoro-3-(trifluoromethyl)phenyl)thiazol-2-yl)benzamide (from Example 72) and 2-(methylamino)ethanol were converted to the title compound. LC-MS m/z (M+H)⁺ 533, (M−H)⁻ 531.

References to other documents, such as patents, patent applications, journals, books, etc., have been made throughout this disclosure. All such documents are hereby incorporated herein by reference in their entirety for all purposes.

It is to be understood that the foregoing description is exemplary and explanatory in nature, and is intended to illustrate the presently disclosed general inventive concept and its preferred embodiments. Through routine experimentation, those of skill in the art given the benefit of the present disclosure may recognize apparent modifications and variations without departing from the spirit and scope of the present disclosure. Thus, the present disclosure is not limited by the above description, but rather by the following claims and their equivalents.

1. A compound of the Formula 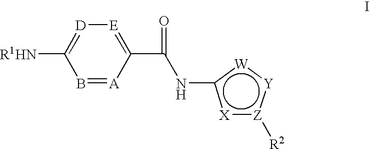
or the pharmaceutically acceptable salts thereof; wherein
R¹ is (C₂-C₉)heteroaryl or (C₂-C₉)heterocycloalkyl wherein the heteroaryl or heterocycloalkyl groups are optionally substituted by one to three groups selected from the group consisting of halo, cyano, nitro, carboxy, hydroxy, amino, NH₂C(O)—, R³(C₁-C₆)alkyl, R³(C₁-C₆)alkoxy, R³(C₁-C₆)alkoxycarbonyl, R³(C₁-C₆)alkylthio, R³(C₁-C₆)alkylsulfinyl, R³(C₁-C₆)alkylsulfonyl, R³(C₁-C₆)alkylaminosulfonyl, R³(C₁-C₆)alkylsulfonylamino, R³(C₁-C₆)alkylamino, R³(C₁-C₆)alkylcarboxy, R³(C₁-C₆)alkyl-NH—C(O)—, amino-C(O)—NH—, R³(C₁-C₆)alkylamino C(O)—NH—, aminocarbonyl(C₁-C₆)alkyl, R³(C₁-C₆)alkylaminocarbonyl(C₁-C₆)alkyl, amino-C(O)—O—, amino(C₁-C₆)alkoxycarbonyl, R³(C₁-C₆)alkylamino-C(O)—O—, R³(C₁-C₆)alkylamino(C₁-C₆)alkoxycarbonyl, R³(C₁-C₆)alkoxy-C(O)—NH—, R³(C₁-C₆)alkoxy(C₁-C₆)alkylamino, trifluoromethyl, trifluoromethyl(C₁-C₆)alkyl, R³(C₁-C₆)alkyl-CF₂, trifluoromethyl[(C₁-C₆)alkyl]ₐ-(CF₂)_{b}—[(C₁-C₆)alkyl]_{c}- wherein a is 0 or 1, b is 1, 2, 3 or 4, and c is 0 or 1; R⁴R⁵N—, R⁴R⁵N—C(O)—, R⁴R⁵N—C(O)—NH—, R⁴R⁵N—C(O)—(C₁-C₆)alkyl and R⁴R⁵N—C(O)—O—;
wherein R³ is one to three groups selected from hydrogen, (C₁-C₆)alkoxy, hydroxy, carboxy, amino, (C₁-C₆)alkylamino, R⁴R⁵N—, (C₁-C₆)alkylamino, (C₁-C₆)alkylthio, (C₁-C₆)alkylsulfinyl, (C₁-C₆)alkylsulfonyl and NH₂—C(O)—;
R⁴ and R⁵ are each independently (C₁-C₆)alkyl optionally substituted by (C₁-C₆)alkoxy, hydroxy, carboxy, amino, (C₁-C₆)alkylamino, amino, (C₁-C₆)alkylamino, ((C₁-C₆)alkyl)₂amino, (C₁-C₆)alkylthio, (C₁-C₆)alkylsulfinyl, (C₁-C₆)alkylsulfonyl and NH₂—C(O)—;
or R⁴ and R⁵ may be taken together with the nitrogen to which they are attached to form a 4 to X membered ring wherein the 6 to 8 membered rings may further optionally contain one to three heteroatoms selected from the group consisting of O, S, S(O), S(O)₂, NH or ((C₁-C₆)alkyl)-N—; and the ring so formed is optionally substituted by (C₁-C₆)alkoxy, hydroxy, carboxy, amino, (C₁-C₆)alkylamino, amino, (C₁-C₆)alkylamino, ((C₁-C₆)alkyl)₂amino, (C₁-C₆)alkylthio, (C₁-C₆)alkylsulfinyl, (C₁-C₆)alkylsulfonyl and NH₁₂—C(O)—;
A, B, D, F are each independently CH, N or CR⁶ wherein R¹ is halo, cyano, nitro, carboxy, hydroxy, amino, NH₂C(O)—, R⁷(C₁-C₆)alkyl, R⁷(C₁-C₆)alkoxy, R⁷(C₁-C₆)alkoxycarbonyl, R⁷(C₁-C₆)alkylthio, R⁷(C₁-C₆)alkylsulfonyl, R⁷(C₁-C₆)alkylsulfonyl, R⁷(C₁-C₆)alkylaminosulfonyl, R⁷(C₁-C₆)alkylsulfonylamino, R⁷(C₁-C₆)alkylamino, R⁷(C₁-C₆)alkylcarboxy, R⁷(C₁-C₆)alkyl-NH—C(O)—, amino-C(O)—NH—, R⁷(C₁-C₆)alkylamino C(O)—NH—, aminocarbonyl(C₁-C₆)alkyl, R⁷(C₁-C₆)alkylaminocarbonyl(C₁-C₆)alkyl, amino-C(O)—O—, amino(C₁-C₆)alkoxycarbonyl, R⁷(C₁-C₆)alkylamino-C(O)—O—, R⁷(C₁-C₆)alkylamino(C₁-C₆)alkoxycarbonyl, R⁷(C₁-C₆)alkoxy-C(O)—NH—, R⁷(C₁-C₆)alkoxy(C₁-C₆)alkylamino, trifluoromethyl, trifluoromethyl(C₁-C₆)alkyl, R⁷(C₁-C₆)alkyl-CF₂, trifluoromethyl[(C₁-C₆)alkyl]ₐ-(CF₂))_{b}-[(C₁-C₆)alkyl]_{c}- wherein a is 0 or 1, b is 1, 2, 3 or 4, and c is 0 or 1; R⁸R⁹N—, R⁸R⁹N—C(O)—, R⁸R⁹—C(O)—NH—, R⁸R⁹N—C(O)—(C₁-C₆)alkyl and R⁸R⁸R⁹N—C(O)—O—;
wherein R⁷ is one to three groups selected from hydrogen, (C₁-C₆)alkoxy, hydroxy, carboxy, amino, (C₁-C₆)alkylamino, R⁸R⁹N—, (C₁-C₆)alkylamino, (C₁-C₆)alkylthio, (C₁-C₆)alkylsulfinyl, (C₁-C₆)alkylsulfonyl or NH₂—C(O)—;
R⁸ and R⁹ are each independently (C₁-C₆)alkyl optionally substituted by (C₁-C₆)alkoxy, hydroxy, carboxy, amino, (C₁-C₆)alkylamino, amino, (C₁-C₆)alkylamino, ((C₁-C₆)alkyl)₂amino, (C₁-C₆)alkylthio, (C₁-C₆)alkylsulfinyl, (C₁-C₆)alkylsulfonyl and NH₂—C(O)—;
or R⁸ and R⁹ may be taken together with the nitrogen to which they are attached to form a 4 to 8 membered ring wherein the 6 to 8 membered rings may further optionally contain one to three heteroatoms selected from the group consisting of O, S, S(O), S(O)₂, NH or ((C₁-C₆)alkyl)-N—; and the ring so formed is optionally substituted by (C₁-C₆)alkoxy, hydroxy, carboxy, amino, (C₁-C₆)alkylamino, amino, (C₁-C₆)alkyl amino, ((C₁-C₆)alkyl)₂amino, (C₁-C₆)alkylthio, (C₁-C₆)alkylsulfinyl, (C₁-C₆)alkylsulfonyl and NH₂—C(O)—;
W, X and Y are each independently selected from the group consisting of C, CH, CR¹⁰, O, S, N, NH and R¹⁰(((C₁-C₆)alkyl)-N;
Z is C or N;
wherein R¹⁰ is halo, cyano, nitro, carboxy, hydroxy, amino, NH₂C(O)—, R¹¹(C₁-C₆)alkyl, R¹¹(C₁-C₆)alkoxy, R¹¹(C₁-C₆)alkoxycarbonyl, R¹¹(C₁-C₆)alkylthio, R¹¹(C₁-C₆)alkylsulfinyl, R¹¹(C₁-C₆)alkylsulfonyl, R¹¹(C₁-C₆)alkylaminosulfonyl, R¹¹(C₁-C₆)alkylsulfonylamino, R¹¹(C₁-C₆)alkylamino, R¹¹(C₁-C₆)alkylcarboxy, R¹¹(C₁-C₆)alkyl-N—-C(O)—, amino-C(O)—NH—, R¹¹ (C₁-C₆)alkylamino C(O)—NH—, aminocarbonyl(C₁-C₆)alkyl, R¹¹(C₁-C₆)alkylaminocarbonyl(C₁-C₆)alkyl, amino-C(O)—O—, amino(C₁-C₆)alkoxycarbonyl, R¹¹(C₁-C₆)alkylamino-C(O)—O—, R¹¹(C₁-C₆)alkylamino(C₁-C₆)alkoxycarbonyl, R¹¹(C₁-C₆)alkoxy-C(O)—NH—, R^{11K}(C₁-C₆)alkoxy(C₁-C₆)alkylamino, trifluoromethyl, trifluoromethyl(C₁-C₆)alkyl, R¹¹(C₁-C₆)alkyl-CF₂, trifluoromethyl[(C₁-C₆)alkyl]ₐ—(CF₂)_{b}-[C₁-C₆)alkyl]_{c}-, wherein a is 0 or 1, b is 1, 2, 3 or 4, and c is 0 or 1; R¹²R¹³N—, R¹²R¹³N—C(O)—, R¹²R¹³N—C(O)—NH—, R¹²R¹³N—C(O)—(C₁-C₆)alkyl and R¹²R¹³N—C(O)—O—;
wherein R¹¹ is one to three groups selected from hydrogen, (C₁-C₆)alkoxy, hydroxy, carboxy, amino, (C₁-C₆)alkylamino, R¹²R¹³N—, (C₁-C₆)alkylamino, (C₁-C₆)alkylthio, (C₁-C₆)alkylsulfinyl, (C₁-C₆)alkylsulfonyl and NH₂—C(O)—;
R¹² and R¹³ are each independently (C₁-C₆)allyl optionally substituted by (C₁-C₆)alkoxy, hydroxy, carboxy, amino, (C₁-C₆)alkylamino, amino, (C₁-C₆)alkylamino, ((C₁-C₆)alkyl)₂amino, (C₁-C₆)alkylthio, (C₁-C₆)alkylsulfinyl, (C₁-C₆)alkylsulfonyl and NH₂—C(O)—;
or R¹² and R¹³ may be taken together with the nitrogen to which they are attached to form a 4 to 8 membered ring wherein the 6 to 8 membered rings may further optionally contain one to three heteroatoms selected from the group consisting of O, S, S(O), S(O)₂, NH or ((C₁-C₆)alkyl)-N—; and the ring so formed is optionally substituted by (C₁-C₆)alkoxy, hydroxy, carboxy, amino, (C₁-C₆)alkylamino, amino, (C₁-C₆)alkylamino, ((C₁-C₆)alkyl)₂amino, (C₁-C₆)alkylthio, (C₁-C₆)alkylsulfinyl, (C₁-C₆)alkylsulfonyl and NH₂—C(O)—; and
R² is R¹⁴(C₆-C₁₀)aryl, R¹⁴(C₂-C₉)heteroaryl, R¹⁴(C₃-C₁₀)cycloalkyl or R¹⁴(C₂-C₉)heterocycloalkyl; wherein R¹⁴ is one to three groups selected from hydrogen, halo, cyano, nitro, carboxy hydroxy, amino, NH₂C(O)—, R¹⁵(C₁-C₆)alkyl, R¹⁵(C₃-C₁₀)cycloalkyl, R¹⁵(C₁-C₆)alkoxy, R¹⁵(C₁-C₆)alkoxycarbonyl, R¹⁵(C₁-C₆)alkylthio, R¹⁵(C₁-C₆)alkylsulfinyl, R¹⁵(C₁-C₆)alkylsulfonyl, R¹⁵(C₁-C₆)alkylaminosulfonyl, R¹⁵(C₁-C₆)alkylsulfonylamino, R¹⁵(C₁-C₆)alkylamino, R¹⁵(C₁-C₆)alkylcarboxy, R¹⁵(C₁-C₆)alkyl-NH—C(O)—, amino-C(O)—NH—, R¹⁵(C₁-C₆)alkylamino C(O)—NH—, aminocarbonyl(C₁-C₆)alkyl, R¹⁵(C₁-C₆)alkylaminocarbonyl(C₁-C₆)alkyl, amino-C(O)—O—, amino(C₁-C₆)alkoxycarbonyl, R¹⁵(C₁-C₆)alkylamino-C(O)—O—, R¹⁵ (C₁-C₆)alkylamino(C₁-C₆)alkoxycarbonyl, R¹⁵(C₁-C₆)alkoxy-C(O)—NH—, R¹⁵(C₁-C₆)alkoxy(C₁-C₆)alkylamino, trifluoromethyl, trifluoromethoxy, trifluoromethyl(C₁-C₆)alkyl, R¹⁵(C₁₋C₆)alkyl-CF₂, trifluoromethyl[(C₁-C₆)alkyl]ₐ-(CF₂)_{b}[(C₁-C₆)alkyl]_{c}-, wherein a is 0 or 1, b is 1, 2, 3 or 4, and c is 0 or 1 R¹⁶R¹⁷N—, R¹⁶R¹⁷N—C(O)—, R¹⁶R¹⁷N—C(O)—NH—, R¹⁶R¹⁷N—C(O)—(C₁-C₆)alkyl and R¹⁶R¹⁷N—C(O)—O—;
wherein R¹⁵ is one to three groups selected from hydrogen, (C₁-C₆)alkyl, (C₃-C₁₀)cycloalkyl, (C₁-C₆)alkoxy, hydroxy, cyano, carboxy, amino, (C₁-C₆)alkylamino, R¹⁶R¹⁷N—, (C₁-C₆)alkylamino, (C₁-C₆)alkylthio, (C₁-C₆) alkylsulfinyl, (C₁-C₆)alkylsulfonyl and NH₂—C(O)—;
R¹⁶ and R¹⁷ are each independently (C₁-C₆)alkyl optionally substituted by (C₁-C₆)alkoxy, hydroxy, carboxy, amino, (C₁-C₆)alkylamino, amino, (C₁-C₆)alkylamino, ((C₁-C₆)alkyl)₂amino, (C₁-C₆)alkylthio, (C₁-C₆)alkylsulfinyl, (C₁-C₆)alkylsulfonyl and NH₂—C(O)—;
or R¹⁶ and R¹⁷ may be taken together with the nitrogen to which they are attached to form a 4 to 8 membered ring wherein the 6 to 8 membered rings may further optionally contain one to three heteroatoms selected from the group consisting of O, S, S(O), S(O)₂, NH or ((C₁-C₆)alkyl)-N—; and the ring so formed is optionally substituted by (C₁-C₆)alkoxy, hydroxy, carboxy, amino, (C₁-C₆)alkylamino, amino, (C₁-C₆)alkylamino, ((C₁-C₆)alkyl)₂amino, (C₁-C₆)alkylthio, (C₁-C₆)alkylsulfinyl, (C₁-C₆)alkylsulfonyl and NH₂—C(O)—.

2. A compound according to claim 1, wherein R¹ is (C₂-C₉)heteroaryl optionally substituted by one to three groups selected from the group consisting of: R³(C₁-C₆)alkyl, R³(C₁-C₆)alkoxy, R³(C₁-C₆)alkylthio, R³(C₁-C₆)alkylamino and R³(C₁-C₆)alkoxy(C₁-C₆)alkylamino, wherein R³ is defined as above.

3. A compound according to claim 2, wherein R¹ is pyridine, pyrazine, pyrimidine, pyridazine, 1,3,5-triazine, 1,2,4-triazine or 1,2,3-triazine.

4. A compound according to claim 1, wherein R² is phenyl, pyridinyl, pyrimidinyl, pyrazinyl or pyridazinyl, each optionally substituted by a group selected from: cyano, hydroxy, amino, R¹⁵(C₁-C₆)alkyl, R¹⁵(C₁-C₆)alkoxy, R¹⁵(C₁-C₆)alkylthio, R¹⁵(C₃-C₁₀)cycloalkyl or trifluoromethoxy, wherein R¹⁵ is defined as above.

5. A compound according to claim 1, wherein:
X is N or CH, W is O, S or NH, Y is N or CH, and Z is C;
X is O, S or N, W is N or CH, Y is N or CH, and Z is C;
X is N or CH, W is N or CH, Y is O, S or NH, and/is C;
N is N or CU, W is N or CH, Y is N or CH, and Z is N;
X is N, W is S, Y is CH, and Z is C;
X is N, W is S, Y is N, and Z is C;
A is N, B is C, D is CH, and E is CH;
A is CH, 13 is N, D is CH, and E is CH;
A is N, B is CH, D is CH, and E is N;
A is N, B is CH, D is N, and E is CH;
A is N, B is N, D is CH, and E is CH;
A is N, B is N, D is N, and E is CH;
A is N B is CH, D is N, and E is N; or
A is CH, B is CH, D is CH, and E is CH.

6. A compound according to claim 1, wherein R¹ is pyridine, pyrazine, pyrimidine, pyridazine, 1,3,5-triazine, 1,2,4-triazine or 1,2,3-triazine; R² is phenyl, pyridinyl, pyrimidinyl, pyrazinyl or pyridazinyl; X is N; W is S; Y is CH; Z is C; A is CH; B is CH; D is CH and E is CR.

7. A compound according to claim 1, wherein R¹ is pyridine, pyrazine, pyrimidine, pyridazine, 1,3,5-triazine, 1,2,4-triazine or 1,2,3-triazine; R² is phenyl, pyridinyl, pyrimidinyl, pyrazinyl and pyridazinyl; X is N; W is S; Y is N; Z is C; A is CH; B is CH; D is CH and E is CH.

8. A compound according to claim 1 selected from the group consisting of:
N-[2-(4-Fluoro-3-trifluoromethyl-phenyl)-oxazol-4-yl]-4-(pyrimidin-4-ylamino)-benzamide;
N-[2-(2-Fluoro-3-trifluoromethyl-phenyl)-oxazol-4-yl]-4-(pyrimidin-4-ylamino)-benzamide;
N-[2-(2,4-Difluoro-phenyl)-thiazol-4-yl]-4-(pyrimidin-4-ylamino)-benzamide;
N-[2-(2-Fluoro-3-trifluoromethyl-phenyl)-thiazol-4-yl]-4-(pyrimidin-4-ylamino)-benzamide;
N-[2-(4-Fluoro-3-trifluoromethyl-phenyl)-thiazol-4-yl]-4-(pyrimidin-4-ylamino)-benzamide;
N-[3-(4-Fluoro-3-trifluoromethyl-phenyl)-[1,2,4]thiadiazol-5-yl]-4-(pyrimidin-4-ylamino)-benzamide;
N-[3-(2,4-Difluoro-phenyl)-[1,2,4]thiadiazol-5-yl]-4-pyrimidin-4-ylamino)-benzamide;
N-[3-(2-Fluoro-3-trifluoromethyl-phenyl)-[1,2,4]thiadiazol-5-yl]-4-(pyrimidin-4-ylamino)-benzamide;
N-(3-Phenyl-[1,2,4]thiadiazol-5-yl)-4-(pyrimidin-4-ylamino)-benzamide;
4-(6-Azetidin-1-yl-pyrimidin-4-ylamino)-N-[3-(4-fluoro-3-trifluoromethyl-phenyl)-[1,2,4]thiadiazol-5-yl]-benzamide;
4-(6-Dimethylamino-pyrimidin-4-ylamino)-N-[3-(4-fluoro-3-trifluoromethyl-phenyl)-[1,2,4]thiadiazol-5-yl]-benzamide;
N-[3-(3-Trifluoromethoxy-phenyl)-[1,2,4]thiadiazol-5-yl]-4-(pyrimidin-4-ylamino)-benzamide;
N-[3-(3,4-Dichloro-phenyl)-[1,2,4]thiadiazol-5-yl]-4-(pyrimidin-4-ylamino)-benzamide;
N-[3-(3-Fluoro-4-trifluoromethyl-phenyl)-[1,2,4]thiadiazol-5-yl]-4-(pyrimidin-4-ylamino)-benzamide;
N-[3-(3,4-Difluoro-phenyl)-[1,2,4]thiadiazol-5-yl]-4-(pyrimidin-4-ylamino)-benzamide;
N-[5-(4-Fluoro-3-trifluoromethyl-phenyl)-[1,2,4]thiadiazol-3-yl]-4-(pyridazin-4-ylamino)-benzamide;
N-[5-(3,4-Difluoro-phenyl)-[1,2,4]thiadiazol-3-yl]-4-(pyrimidin-4-ylamino)-benzamide;
N-[4-(2-Fluoro-3-trifluromethyl-phenyl)-thiazol-2-yl]-4-(pyrimidin-4-ylamino)-benzamide;
N-[4-(Phenyl)-thiazol-2-yl]-4-(pyrimidin-4-ylamino)-benzamide;
N-[4-(2-Fluoro-phenyl)-thiazol-2-yl]-4-(pyrimidin-4-ylamino)-benzamide;
N-[4-(3-Fluoro-phenyl)-thiazol-2-yl]-4-(pyrimidin-4-ylamino)-benzamide;
N-[4-(4-Fluoro-phenyl)-thiazol-2-yl]-4-(pyrimidin-4-ylamino)-benzamide;
N-[4-(2,3-Difluoro-phenyl)-thiazol-2-yl]-4-pyrimidin-4-ylamino)-benzamide;
N-[4-(2,4-Difluoro-phenyl)-thiazol-2-yl]-4-(pyrimidin-4-ylamino)-benzamide;
N-[4-(2,6-Difluoro-phenyl)-thiazol-2-yl]-4-(pyrimidin-4-ylamino)-benzamide;
N-[4-(3,4-Difluoro-phenyl)-thiazol-2-yl]-4-(pyrimidin-4-ylamino)-benzamide;
N-[4-(2-Chloro-phenyl)-thiazol-2-yl]-4-(pyrimidin-4-ylamino)-benzamide;
N-[4-(3-Chloro-phenyl)-thiazol-2-yl]-4-(pyrimidin-4-ylamino)-benzamide;
N-[4-(4-Chloro-phenyl)-thiazol-2-yl]-4-(pyrimidin-4-ylamino)-benzamide;
N-[4-(2,3-Dichloro-phenyl)-thiazol-2-yl]-4-(pyrimidin-4-ylamino)-benzamide;
N-[4-(2,4-Dichloro-phenyl)-thiazol-2-yl]-4-(pyrimidin-4-ylamino)-benzamide;
N-[4-(3,4-Dichloro-phenyl)-thiazol-2-yl]-4-(pyrimidin-4-ylamino)-benzamide;
N-[4-(2-Trifluoromethyl-phenyl)-thiazol-2-yl]-4-(pyrimidin-4-ylamino)-benzamide;
N-[4-(3-Trifluoromethyl-phenyl)-thiazol-2-yl]-4-(pyrimidin-4-ylamino)-benzamide;
N-[4-(4-Trifluoromethyl-phenyl)-thiazol-2-yl]-4-(pyrimidin-4-ylamino)-benzamide;
N-[4-(2-Fluoro-3-chloro-phenyl)-thiazol-2-yl]-4-(pyrimidin-4-ylamino)-benzamide;
N-[4-(2-Fluoro-4-chloro-phenyl)-thiazol-2-yl]-4-(pyrimidin-4-ylamino)-benzamide;
N-[4-(2-Fluoro-4-trifluoromethyl-phenyl)-thiazol-2-yl]-4-(pyrimidin-4-ylamino)-benzamide:
N-[4-(2-Fluoro-5-trifluoromethyl-phenyl)-thiazol-2-yl]-4-(pyrimidin-4-ylamino)-benzamide;
N-[4-(4-Fluoro-3-trifluoromethyl-phenyl)-thiazol-2-yl]-4-(pyrimidin-4-ylamino)-benzamide;
N-[4-(3-Fluoro-4-trifluoromethyl-phenyl)-thiazol-2-yl]-4-(pyrimidin-4-ylamino)-benzamide;
N-[4-(2,6-Dichloro-4-trifluoromethyl-phenyl)-thiazol-2-yl]-4-(pyrimidin-4-ylamino)-benzamide;
N-[4-(4-Methyl-phenyl)-thiazol-2-yl]-4-(pyrimidin-4-ylamino)-benzamide;
N-[4-(2,4-Dimethyl-phenyl)-thiazol-2-yl]-4-(pyrimidin-4-ylamino)-benzamide;
N-[4-(4-Difluoromethoxy-phenyl)-thiazol-2-yl]-4-(pyrimidin-4-ylamino)-benzamide;
N-[4-(3-Trifluoromethoxy-phenyl)-thiazol-2-yl]-4-(pyrimidin-4-ylamino)-benzamide;
N-[4-(4-Trifluoromethoxy-phenyl)-thiazol-2-yl]-4-(pyrimidin-4-ylamino)-benzamide;
N-[4-(2-Naphthyl)-thiazol-2-yl]-4-(pyrimidin-4-ylamino)-benzamide;
4-(Pyrimidin-4-ylamino)-N-[4-(3-bromo-phenyl)-thiazol-2-yl]-benzamide;
4-(Pyrimidin-4-ylamino)-NT-[4-(2-fluoro-3-bromo-phenyl)-thiazol-2-yl]-benzamide;
4-(Pyrimidin-4-ylamino)-N-[4-(2,3,4-trifluoro-phenyl)-thiazol-2-yl]-benzamide;
4-(Pyrimidin-4-ylamino)-N-[4-(2,3-difluoro-4-trifluoromethyl-phenyl)-thiazol-2-yl]-benzamide;
4-(Pyrimidin-4-ylamino)-N-[4-(6-trifluoromethyl-pyridin-2-yl)-thiazol-2-yl]-benzamide;
4-(Pyrimidin-4-ylamino)-N-[4-(2-fluoro-3-trifluoromethoxy-phenyl)-thiazol-2-yl]-benzamide;
4-(Pyridimin-2-ylamino)-N-[4-(2-fluoro-3-trifluoromethyl-phenyl)-thiazol-2-yl]-benzamide;
4-(Pyridin-4-ylamino)-N-[4-(2-fluoro-3-trifluoromethyl-phenyl)-thiazol-2-yl]-benzamide;
4-(Pyridin-4-ylamino)-N-[4-(2-fluoro-3-trifluoro-phenyl)-thiazol-2-yl]-benzamide;
4-(Pyridazin-4-ylamino)-N-[4-(2-fluoro-3-trifluoromethyl-phenyl)-thiazol-2-yl]-benzamide;
4-(Pyridazin-4-ylamino)-N-[4-(4-chloro-phenyl)-thiazol-2-yl]-benzamide;
4-(Pyridazin-4-ylamino)-N-[4-(2,4-difluoro-phenyl)-thiazol-2-yl]-benzamide;
4-(Pyridazin-4-ylamino)-N-[4-fluoro-3-trifluoromethyl-phenyl)-thiazol-2-yl]-benzamide;
4-(Pyrazin-2-ylamino)-N-[4-(2-fluoro-3-trifluoromethyl-phenyl)-thiazol-2-yl]-benzamide;
4-(Pyridazin-4-ylamino)-N-[4-(4-fluoro-3-trifluoromethyl-phenyl)-thiazol-2-yl]-benzamide;
4-(1,35-Triazin-2-ylamino)-N-[4-(4-fluoro-3-trifluoromethyl-phenyl)-thiazol-2-yl]-benzamide;
N-[4-(2-Fluoro-3-trifluoromethyl-phenyl)-thiazol-2-yl]-4-(pyrimidin-5-ylamino)-benzamide;
N-[4-(2-Fluoro-3-trifluoromethyl-phenyl)-thiazol-2-yl]-4-(pyridazin-3-ylamino)-benzamide;
N-[4-(2-Fluoro-3-trifluoromethyl-phenyl)-thiazol-2-yl]-4-(1,3,4-triazin-3-ylamino)-benzamide;
4-(6-Dimethylamino-pyrimidin-4-ylamino)-N-[4-(2-fluoro-3-trifluoromethyl-phenyl)-thiazol-2-yl]-benzamide;
4-(6-Azetidin-1-yl-pyrimidin-4-ylamino)-N-[4-(2-fluoro-3-trifluoromethyl-phenyl)-thiazol-2-yl]-benzamide;
N-[4-(2-Fluoro-3-trifluoromethyl-phenyl)-thiazol-2-yl]-4-(6-pyrrolidin-1-yl-pyrimidin-4-ylamino)-benzamide;
4-(6-Dimethylamino-pyrimidin-4-ylamino)-N-[4-(2-fluoro-3-trifluoromethyl-phenyl)-thiazol-2-yl]-benzamide;
4-(6-Chloro-pyrimidin-4-ylamino)-N-[4-(2-fluoro-3-trifluoromethyl-phenyl)-thiazol-2-yl]-benzamide;
4-(6-Chloro-pyrimidin-4-ylamino)-N-[4-(4-fluoro-3-trifluoromethyl-phenyl)-thiazol-2-yl]-benzamide;
4-(6-Ethylamino-pyrimidin-4-ylamino)-N-[4-(2-fluoro-3-trifluoromethyl-phenyl)-thiazol-2-yl]-benzamide;
4-(6-Cyclopropylamino-pyrimidin-4-ylamino)-N-[4-(2-fluoro-3-trifluoromethyl-phenyl)-thiazol-2-yl]-benzamide;
N-[4-(2-Fluoro-3-trifluoromethyl-phenyl)-thiazol-2-yl]-4-(6-piperidin-1-yl-pyrimidin-4-ylamino)-benzamide;
N-[4-(2-Fluoro-3-trifluoromethyl-phenyl)-thiazol-2-yl]-4-(6-methyl-pyrimidin-4-ylamino)-benzamide;
4-(2,6-Dichloro-pyrimidin-4-ylamino)-N-[4-(2-fluoro-3-trifluoromethyl-phenyl)-thiazol-2-yl]-benzamide;
4-(2-Chloro-6-methyl-pyrimidin-4-ylamino)-N-[4-(2-fluoro-3-trifluoromethyl-phenyl)-thiazol-2-yl]-benzamide;
4-(2,6-Dimethyl-pyrimidin-4-ylamino)-N-[4-(2-fluoro-3-trifluoromethyl-phenyl)-thiazol-2-yl]-benzamide;
4-(2,6-Dimethyl-pyrimidin-4-ylamino)-N-[4-(3,4-difluoro-phenyl)-thiazol-2-yl]-benzamide;
4-(6-Chloropyrimidin-4-ylamino)-N-(3-(4-fluoro-3-(trifluoromethyl)phenyl)-1,2,4-thiadiazol-5-yl)benzamide;
4-(6-N-(2-Methoxyethyl)-N-methylamino)pyramid-4-ylamino)-N-(3-(4-fluoro-3-(trifluoromethyl)phenyl)-1,2,4-thiadiazol-5-yl)benzamide;
4-(6-N-(2-Methoxyethyl)-N-methylamino)pyrimidin-4-ylamino)-N-(4-(2-fluoro-3-(trifluoromethyl)phenyl)thiazol-2-yl)benzamide;
N-(4-(2,4-Difluoro-3-(trifluoromethyl)phenyl)thiazol-2-yl)-4-(pyrimidin-4-ylamino)benzamide;
N-(5-(2-Fluoro-3-(trifluoromethoxy)phenyl)-1,2,4-thiadiazol-3-yl)-4-(pyrimidin-4-ylamino)benzamide;
N-(3-(2-Fluoro-5-(trifluoromethoxy)phenyl)-1,2,4-thiadiazol-5-yl)-4-(pyrimidin-4-ylamino)benzamide;
4-(6-(Dimethylamino)pyrimidin-4-ylamino)-N-(3-(2-fluoro-5-(trifluoromethoxy)phenyl)-1,2,4-thiadiazol-5-yl)benzamide;
N-(4-(2-Fluoro-3-methoxyphenyl)thiazol-2-yl)-4-(pyrimidin-4-ylamino)benzamide;
4-(6-(N-Methoxy-N-methylamino)pyrimidin-4-ylamino)-N-(3-(4-fluoro-3-(trifluoromethyl)phenyl)-1,2,4-thiadiazol-5-yl)benzamide;
4-(6-(Methylamino)pyrimidin-4-ylamino)-N-(3-(4-fluoro-3-(trifluoromethyl)phenyl)-1,2,4-thiadiazol-5-yl)benzamide;
N-(3-Phenylisothiazol-5-yl)-4-(pyrimidin-4-ylamino)benzamide;
N-(3-(4-Chlorophenyl)isothiazol-5-yl)-4-(pyrimidin-4-ylamino)benzamide;
N-(3-(3,4,5-Trifluorophenyl)-1,2,4-thiadiazol-5-yl)-4-(pyrimidin-4-ylamino)benzamide;
N-(3-(3-Chloro-4-fluorophenyl)-1,2,4-thiadiazol-5-yl)-4-(pyrimidin-4-ylamino)benzamide;
N-(3-(2-Fluoro-5-(trifluoromethyl)phenyl)-1,2,4-thiadiazol-5-yl)-4-(pyrimidin-4-ylamino)benzamide;
N-(3-(2-Fluoro-3-(trifluoromethoxy)phenyl)-1,2,4-thiadiazol-5-yl)-4-(pyrimidin-4-ylamino)benzamide;
4-(6-((S)-2-Hydroxy-2-((S)-2,2-dimethyl-1,3-dioxolan-4-yl)ethylamino)pyrimidin-4-ylamino)-N-(3-(4-fluoro-3-(trifluoromethyl)phenyl)-1,2,4-thiadiazol-5-yl)benzamide;
4-(6-((2S,3S)-2,3,4-Trihydroxybutylamino)pyrimidin-4-ylamino)-N-(3-(4-fluoro-3-(trifluoromethyl)phenyl)-1,2,4-thiadiazol-5-yl)benzamide;
4-(6-(N-(2-(Dimethylaminoethyl)-N-methylamino)pyrimidin-4-ylamino)-N-(3-(4-fluoro-3-(trifluoromethyl)phenyl)-1,2,4-thiadiazol-5-yl)benzamide;
4-(6-(N-(2-(Hydroxyethyl)-N-methylamino)pyrimidin-4-ylamino)-N-(3-(4-fluoro-3-(trifluoromethyl)phenyl)-5,2,4-thiadiazol-5-yl)benzamide;
4-(6-N-(2,3-(Dihydroxypropyl)-N-methylamino)pyrimidin-4-ylamino)-N-(3-(4-fluoro-3-(trifluoromethyl)phenyl)-1,2,4-thiadiazol-5-yl)benzamide;
4-(6-N-(2,3-(Dihydroxypropyl)-amino)pyrimidin-4-ylamino)-N-(3-(4-fluoro-3-(trifluoromethyl)phenyl)-1,2,4-thiadiazol-5-yl)benzamide;
4-{6-[Bis-(2-hydroxy-ethyl)-amino]-pyrimidin-4-ylamino}-N-[3-(4-fluoro-3-trifluoromethyl-phenyl)-[1,2,4]thiadiazol-5-yl]-benzamide;
4-(6-(1,3-Dihydroxypropan-2-ylamino)pyrimidin-4-ylamino)-N-(3-(4-fluoro-3-(trifluoromethyl)phenyl)-1,2,4-thiadiazol-5-yl)benzamide;
4-(6-((R)-1-Hydroxypropan-2-ylamino)pyrimidin-4-ylamino)-N-(3-(4-fluoro-3-(trifluoromethyl)phenyl)-1,2,4-thiadiazol-5-yl)benzamide;
4-(6-((S)-1-Hydroxypropan-2-ylamino)pyrimidin-4-ylamino)-N-(3-(4-fluoro-3-(trifluoromethyl)phenyl)-1,2,4-thiadiazol-5-yl)benzamide;
4-{6-[Bis-(3-hydroxy-propyl)-amino]-pyrimidin-4-ylamino}-N-[3-(4-fluoro-3-trifluoromethyl-phenyl)-[1,2,4]thiadiazol-5-yl]-benzamide;
4-(6-(3-Hydroxypropylamino)pyrimidin-4-ylamino)-N-(3-(4-fluoro-3-(trifluoromethyl)phenyl)-1,2,4-thiadiazol-5-yl)benzamide;
4-(6-(2-Hydroxyethylamino)pyrimidin-4-ylamino)-N-(3-(4-fluoro-3-(trifluoromethyl)phenyl)-1,2,4-thiadiazol-5-yl)benzamide;
4-(6-(2,3-Dihydroxypropylthio)pyrimidin-4-ylamino)-N-(3-(4-fluoro-3-(trifluoromethyl)phenyl)-1,2,4-thiadiazol-5-yl)benzamide;
4-{6-[N-(2R and 2S)-(2,3-Dihydroxy-propyl)-N-methyl-amino]-pyrimidin-4-ylamino}-N-[3-(4-fluoro-3-trifluoromethyl-phenyl)-[1,2,4]thiadiazol-5-yl]-benzamide;
4-(2-Methylpyrimidin-4-ylamino)-N-(4-(2-fluoro-3-(trifluoromethyl)phenyl)thiazol-2-yl)benzamide;
N-(3-(3,5-Bis(trifluoromethyl)phenyl)-1,2,4-thiadiazol-5-yl)-4-(pyrimidin-4-ylamino)benzamide;
N-(5-(2-Fluoro-3-(trifluoromethyl)phenyl)thiazol-2-yl)-4-(pyrimidin-4-ylamino)benzamide;
N-(5-(4-Fluoro-3-(trifluoromethyl)phenyl)thiazol-2-yl)-4-pyrimidin-4-ylamino)benzamide;
4-(1,3,4-Thiadiazol-2-ylamino)-N-(4-(2-fluoro-3-(trifluoromethyl)phenyl)thiazol-2-yl)benzamide;
N-[1-(4-Fluoro-3-trifluoromethyl-phenyl)-1H-pyrazol-3-yl]-4-(pyrimidin-4-ylamino)-benzamide;
N-[1-(2-Fluoro-3-trifluoromethyl-phenyl)-3H-pyrazol-3-yl]-4-(pyrimidin-4-ylamino)-benzamide;
4-(6-Cyclopropylmethoxy-pyrimidin-4-ylamino)-N-[3-(fluoro-3-trifluoromethyl-phenyl)-[1,2,4]thiadiazol-5-yl]-benzamide;
4-[6-(2-Benzyloxy-ethoxy)-pyrimidin-4-ylamino]-N-[3-(4-fluoro-3-trifluoromethyl-phenyl)-[1,2,4]thiadiazol-5-yl]-benzamide;
N-[3-(4-Fluoro-3-trifluoromethyl-phenyl)-[1,2,4]thiadiazol-5-yl]-4-(6-methoxy-pyrimidin-4-ylamino)-benzamide;
N-[3-(4-Fluoro-3-trifluoromethyl-phenyl)-[1,2,4]thiadiazol-5-yl]-4-(6-isopropoxy-pyridimin-4-ylamino)-benzamide;
4-(6-Ethoxy-pyrimidin-4-ylamino)-N-[3-(4-fluoro-3-trifluoromethyl-phenyl)-[1,2,4]thiadiazol-5-yl]-benzamide;
N-[3-(4-Fluoro-3-trifluoromethyl-phenyl)-[1,2,4]thiadiazol-5-yl]-4-[6-(2-methoxy-ethoxy)-pyrimidin-4-ylamino]-benzamide;
N-[3-(4-Fluoro-3-trifluoromethyl-phenyl)-[1,2,4]thiadiazol-5-yl]-4-[6-(2-methoxy-1-methyl-ethoxy)-pyrimidin-4-ylamino]-benzamide;
4-(6-Cyclobutylmethoxy-pyrimidin-4-ylamino)-N-[3-(4-fluoro-3-trifluoromethyl-phenyl)-[1,2,4]thiadiazol-5-yl]-benzamide;
N-[3-(4-Fluoro-3-trifluoromethyl-phenyl)-[1,2,4]thiadiazol-5-yl]-4-[2-(2-methoxy-1-methyl-ethoxy)-pyridin-4-ylamino]-benzamide;
4-(6-(Azetidin-3-yloxy)-pyrimidin-4-ylamino-N-[3-(4-fluoro-3-trifluoromethyl-phenyl)-[1,2,4]thiadiazol-5-yl]-benzamide;
4-(6-Cyclohexylmethoxy-pyrimidin-4-ylamino)-N-[3-(4-fluoro-3-trifluoromethyl-phenyl)-[1,2,4]thiadiazol-5-yl]-benzamide;
N-[3-(4-Fluoro-3-trifluoromethyl-phenyl)-[1,2,4]thiadiazol-5-yl]-4-(2-methoxy-pyridin-4-ylamino)-benzamide;
N-[3-(4-Fluoro-3-trifluoromethyl-phenyl)-[1,2,4]thiadiazol-5-yl]-4-[6-(2-hydroxy-ethoxy)-pyrimidin-4-ylamino]-benzamide;
N-[3-(4-Fluoro-3-trifluoromethyl-phenyl)-[1,2,4]thiadiazol-5-yl]-4-(1H-[1,2,3]triazolo[4,5-d]pyrimidin-7-ylamino)-benzamide;
N-[3-(4-Fluoro-3-trifluoromethyl-phenyl)-[1,2,4]thiadiazol-5-yl]-4-(9H-purin-6-ylamino)-benzamide;
N-[3-(4-Fluoro-3-trifluoromethyl-phenyl)-[1,2,4]thiadiazol-5-yl]-4-(1H-[1,2,3]triazolo[4,5-d]pyrimidin-7-ylamino)-benzamide;
4-(2-Chloro-pyridin-4-ylamino)-N-[3-(4-fluoro-3-trifluoromethyl-phenyl)-[1,2,4]thiadiazol-5-yl]-benzamide;
4-(6-Chloro-pyrimidin-4-ylamino)-N-[4-(4-fluoro-3-trifluoromethyl-phenyl)-5-methyl-thiazol-2-yl]-benzamide;
N-[3-(3-Bromo-4-fluoro-phenyl)-[1,2,4]thiadiazol-5-yl]-4-(6-chloro-pyrimidin-4-ylamino)-benzamide;
4-(6-Chloro-pyramid-4-ylamino)-N-[5-methyl-4-(3-trifluoromethyl-phenyl)-thiazol-2-yl]-benzamide;
4-(6-Chloro-pyrimidin-4-ylamino)-N-[4-(2-fluoro-3-trifluoromethyl-phenyl)-5-methyl-thiazol-2-yl]-benzamide;
4-(6-Chloro-pyrimidin-4-ylamino)-N-[4-(2-fluoro-5-trifluoromethyl-phenyl)-5-methyl-thiazol-2-yl]-benzamide;
N-[3-(3-Butyl-4-fluoro-phenyl)-[1,2,4]thiadiazol-5-yl]-4-(6-chloro-pyrimidin-4-ylamino)-benzamide;
4-(6-Isopropoxy-pyrimidin-4-ylamino)-N-[3-(4-isopropoxy-3-trifluoromethyl-phenyl)-[1,2,4]thiadiazol-5-yl]-benzamide;
4-(6-Ethoxy-pyrimidin-4-γ amino)-N-[3-(4-ethoxy-3-trifluoromethyl-phenyl)-[1,2,4]thiadiazol-5-yl]-benzamide,
4-(6-Methoxy-pyrimidin-4-ylamino)-N-[3-(4-methoxy-3-trifluoromethyl-phenyl)-[1,2,4]thiadiazol-5-yl]-benzamide;
N-[3-(4-Fluoro-3-trifluoromethyl-phenyl)-[1,2,4]thiadiazol-5-yl]-4-(6-pyrolidin-1-yl-pyrimidin-4-ylamino)-benzamide;
N-[3-(4-Fluoro-3-trifluoromethyl-phenyl)-[1,2,4]thiadiazol-5-yl]-4-(6-methoxy-pyridin-3-ylamino)-benzamide;
N-[3-(4-Fluoro-3-trifluoromethyl-phenyl)-[1,2,4]thiadiazol-5-yl]-4-(pyridin-4-ylamino)-benzamide;
4-(2-Ethoxy-pyridin-4-ylamino)-N-[3-(4-fluoro-3-trifluoromethyl-phenyl)-[1,2,4]thiadiazol-5-yl]-benzamide;
4-(2-Cyclopropylmethoxy-pyridin-4-ylamino)-N-[3-(4-fluoro-3-trifluoromethyl-phenyl)-[1,2,4]thiadiazol-5-yl]-benzamide;
N-[3-(4-Fluoro-3-trifluoromethyl-phenyl)-[1,2,4]thiadiazol-5-yl]-4-(5-trifluoromethyl-pyridin-2-ylamino)-benzamide;
N-[4-(2-Fluoro-3-trifluoromethyl-phenyl)-5-methyl-thiazol-2-yl]-4-{6-[3-(4-methyl-piperazin-1-yl)-propylamino]-pyrimidin-4-ylamino}-benzamide;
N-[4-(2-Fluoro-3-trifluoromethyl-phenyl)-5-methyl-thiazol-2-yl]-4-{6-[3-(2-oxo-pyrrolidin-1-yl)-propylamino]-pyrimidin-4-ylamino}-benzamide;
4-{6-[(2,3-Dihydroxy-propyl)-methyl-amino]-pyrimidin-4-ylamino}-N-[4-(2-fluoro-3-trifluoromethyl-phenyl)-5-methyl-thiazol-2-yl]-benzamide;
4-(6-Dimethylamino-pyrimidin-4-ylamino)-N-[4-(2-fluoro-3-trifluoromethyl-phenyl)-5-methyl-thiazol-2-yl]-benzamide;
N-[4-(2-Fluoro-3-trifluoromethyl-phenyl)-5-methyl-thiazol-2-yl]-4-(6-methylamino-pyrimidin-4-ylamino)-benzamide;
4-(6-Dimethylamino-pyrimidin-4-ylamino)-N-[5-methyl-4-(3-trifluoromethyl-phenyl)-thiazol-2-yl]-benzamide;
4-[6-(2,3-Dihydroxy-propylamino)-pyrimidin-4-ylamino]-N-[4-(2-fluoro-3-trifluoromethyl-phenyl)-5-methyl-thiazol-2-yl]-benzamide;
4-[6-(4-Ethyl-piperazin-1-yl)-pyrimidin-4-ylamino]-N-[4-(2-fluoro-3-trifluoromethyl-phenyl)-5-methyl-thiazol-2-yl]-benzamide;
4-(6-Methylamino-pyrimidin-4-ylamino)-N-[5-methyl-4-(3-trifluoromethyl-phenyl)-thiazol-2-yl]-benzamide;
4-[6-(2,3-Dihydroxy-propylamino)-pyrimidin-4-ylamino]-N-[5-methyl-4-(3-trifluoromethyl-phenyl)-thiazol-2-yl]-benzamide;
4-[6-(2,3-Dihydroxy-propylamino)-pyrimidin-4-ylamino]-N-[5-methyl-4-(3-trifluoromethyl-phenyl)-thiazol-2-yl]-benzamide;
4-(6-Dimethylamino-pyrimidin-4-ylamino)-N-[5-methyl-4-(3-trifluoromethyl-phenyl)-thiazol-2-yl]-benzamide;
4-(6-Dimethylamino-pyrimidin-4-ylamino)-N-[4-(2-fluoro-5-trifluoromethyl-phenyl)-5-methyl-thiazol-2-yl]-benzamide;
4-{6-[(2,3-Dihydroxy-propyl)-methyl-amino]-pyrimidin-4-ylamino}-N-[4-(2-fluoro-5-trifluoromethyl-phenyl)-5-methyl-thiazol-2-yl]-benzamide;
4-[6-(2,3-Dihydroxy-propylamino)-pyrimidin-4-ylamino]-N-[4-(4-fluoro-3-trifluoromethyl-phenyl)-5-methyl-thiazol-2-yl]-benzamide;
N-[4-(4-Fluoro-3-trifluoromethyl-phenyl)-5-methyl-thiazol-2-yl]-4-(6-methylamino-pyrimidin-4-ylamino)-benzamide;
N-[4-(4-Fluoro-3-trifluoromethyl-phenyl)-5-methyl-thiazol-2-yl]-4-(6-methylamino-pyrimidin-4-ylamino)-benzamide;
N-[4-(4-Fluoro-3-trifluoromethyl-phenyl)-5-methyl-thiazol-2-yl]-4-(6-methylamino-pyrimidin-4-ylamino)-benzamide;
N-[4-(4-Fluoro-3-trifluoromethyl-phenyl)-5-methyl-thiazol-2-yl]-4-{6-[3-(4-methyl-piperazin-1-yl)-propylamino]-pyrimidin-4-ylamino}-benzamide;
4-(6-Dimethylamino-pyrimidin-4-ylamino)-N-[4-(4-fluoro-3-trifluoromethyl-phenyl)-5-methyl-thiazol-2-yl]-benzamide;
4-{6-[(2,3-Dihydroxy-propyl)-methyl-amino]-pyrimidin-4-ylamino}-N-[4-(4-fluoro-3-trifluoromethyl-phenyl)-5-methyl-thiazol-2-yl]-benzamide;
N-[3-(3-Butyl-4-fluoro-phenyl)-[1,2,4]thiadiazol-5-yl]-4-(6-dimethylamino-pyrimidin-4-ylamino)-benzamide;
4-{6-[Bis-(3-hydroxy-propyl)-amino]-pyrimidin-4-ylamino}-N-[3-(3-butyl-4-fluoro-phenyl)-[1,2,4]thiadiazol-5-yl]-benzamide;
2-Butyl-4-[4-(2-{4-[3-(1,3-dimethyl-pentyl)-benzyl]-phenyl}-allyl)-cyclopenta-1,4-dienyl]-1-methyl-benzene;
N-[3-(3-Butyl-4-fluoro-phenyl)-[1,2,4]thiadiazol-5-yl]-4-(6-methylamino-pyrimidin-4-ylamino)-benzamide;
N-[3-(3-Butyl-4-fluoro-phenyl)-[1,2,4]thiadiazol-5-yl]-4-{6-[3-(2-oxo-pyrrolidin-1-yl)-propylamino]-pyrimidin-4-ylamino}-benzamide;
N-[5-Methyl-4-(2-fluoro-3-trifluoromethyl-phenyl)-thiazol-2-yl]-4-(pyrimidin-4-ylamino)-benzamide;
4N-[4(4-Fluoro-3-trifluoromethyl-phenyl)-5-methyl-thiazol-2-yl]-4-(pyrimidin-4-ylamino)-benzamide;
N-[5 ethyl-4-(3-trifluoromethyl-phenyl)-thiazol-2-yl]-4-(pyrimidin-4-ylamino)-benzamide;
N-[3-(3-Bromo-4-fluoro-phenyl)-[1,2,4]thiadiazol-5-yl]-4-(pyrimidin-4-ylamino)-benzamide;
N-[4-(3-ethyl-2-fluoro-phenyl)-thiazol-2-yl]-4-(pyrimidin-4-ylamino)-benzamide;
N-[4-(3-Butyl-2-fluoro-phenyl)-thiazol-2-yl]-4-(pyrimidin-4-ylamino)-benzamide;
N-[4-(2-Fluoro-3-isobutyl-phenyl)-thiazol-2-yl]-4-(pyrimidin-4-ylamino)-benzamide,
N-[3-(3-Butyl-4-fluoro-phenyl)-[1,2,4]thiadiazol-5-yl]-4-(pyrimidin-4-ylamino)-benzamide;
N-[4-(2-Fluoro-5-trifluoromethyl-phenyl)-5-methyl-thiazol-2-yl]-4-(pyrimidin-4-ylamino)-benzamide;
4-(6-(3-Hydroxyazetidin-1-yl)pyrimidin-4-ylamino)-N-(4-(2-fluoro-3-(trifluoromethoxy)phenyl)thiazol-2-yl)benzamide;
4-(6-(3-Hydroxyazetidin-1-yl)pyrimidin-4-ylamino)-N-(3-(4-fluoro-3-(trifluoromethyl)phenyl)-1,2,4-thiadiazol-5-yl)benzamide;
(R)N-[4-(2-fluoro-3-trifluoromethoxy-phenyl)-thiazol-2-yl]-4-{6-[(2,3-dihydroxy-propyl)-methyl-amino]-pyrimidin-4-ylamino}-benzamide;
(S)N-[4-(2-fluoro-3-trifluoromethoxy-phenyl)-thiazol-2-yl]-4-{6-[(2,3-dihydroxy-propyl)-methyl-amino]-pyrimidin-4-ylamino}-benzamide; and
N-[4-(2-fluoro-3-trifluoromethyl-phenyl)-thiazol-2-yl]-4-{6-[(2-hydroxy-ethyl)-methyl-amino]-pyrimidin-4-ylamino}-benzamide
or a pharmaceutically acceptable salt thereof.

9. A pharmaceutical composition for (a) treating or preventing a disorder or condition selected from decreased megakaryopoiesis and platelet numbers, decreased hematopoietic stem cells, decreased erythopoiesis and myelopoiesis; aiding bone marrow repopulation after bone marrow or cord blood transplant; expanding megakaryocyte and stem cell numbers in vitro prior to transplant; increasing platelet numbers in normal individuals prior to surgery, cytoreductive chemotherapy, or radiation treatment; increasing platelet numbers in normal individuals prior to platelet pheresis to harvest platelets for later transfusion; increasing platelet numbers in thrombocytopenic patients or (b) treating or preventing a disorder or condition that can be treated or prevented by agonizing the TPO receptor in a mammal, including a human, comprising an amount of a compound of claim 1 or a pharmaceutically acceptable salt thereof, effective in treating or preventing such disorders or conditions and a pharmaceutically acceptable carrier.

10. A method for agonizing the TPO receptor in a mammal including a human, comprising administering to the mammal an effective amount of a compound of claim 1 or a pharmaceutically acceptable salt thereof.

11. A method for treating or preventing a disorder or condition selected from decreased megakaryopoiesis and platelet numbers, decreased hematopoietic stem cells, decreased erythopoiesis and myelopoiesis; aiding bone marrow repopulation after bone marrow or cord blood transplant; expanding megakaryocyte and stem cell numbers in vitro prior to transplant; increasing platelet numbers in normal individuals prior to surgery, cytoreductive chemotherapy, or radiation treatment; increasing platelet numbers in normal individuals prior to platelet pheresis to harvest platelets for later transfusion, and increasing platelet numbers in thrombocytopenic patients, in a mammal, including a human comprising administering and amount of a compound of claim 1 or a pharmaceutically acceptable salt thereof, effective in treating or preventing the disorders or conditions.

12. The method of claim 11 further comprising co-administering a therapeutically effective amount of an agent selected from the group consisting of: a colony stimulating factor, cytokine, chemokine, interleukin or cytokine receptor agonist or antagonists, soluble receptors, receptor agonists or antagonist antibodies, or small molecules or peptides that act by the same mechanisms as one or more of said agents, wherein the agent is selected from the group consisting of: G-CSF, GM-CSF, TPO, M-CSF, EPO, Gro-beta, IL-11, SCF, FLT3 ligand, LIF, 1L-3, IL-6, IL-1, Progenipoietin, NESP, SD-01, IL-8, or IL-S or a biologically active derivative of any of said agents.

13. A method for enhancing platelet production obtained from a donor comprising administering to the donor prior to platelet pheresis, blood donation or platelet donation a therapeutically effective amount of a compound of claim 1 or a pharmaceutically acceptable salt thereof.

14. A method for enhancing the number of peripheral blood stem cells obtained from a donor comprising administering to the donor prior to leukopheresis a therapeutically effective amount of a compound of claim 1 or a pharmaceutically acceptable salt thereof.

15. The method of claim 14 further comprising co-administering a therapeutically effective amount of a hematopoietic-cell mobilizing agent selected from the group consisting of: a colony stimulating factor, cytokine, chemokine, interleukin or cytokine receptor agonist, adhesion molecule antagonists or antibodies, wherein the mobilizing agent is selected from the group consisting of: G-CSF, CM-CSF, TPO, EPO, Gro-beta, 1L-8, cytoxan, VLA-4 inhibitors, SCF, FLT3 ligand or a biologically active derivative of G-CSF, GM-CSF, TPO, EPO, Gro-beta or 1L-8.
